SEQUENCE LISTING (73 sequences)

SEQ ID NO: 1 - DNA, 20 bp, synthetic construct
  source 1..20: mol_type = other DNA
aaagtttagattagattaga

SEQ ID NO: 2 - DNA, 20 bp, synthetic construct
  source 1..20: mol_type = other DNA
aaagtttagattagattaga

SEQ ID NO: 3 - DNA, 20 bp, synthetic construct
  source 1..20: mol_type = other DNA
aaagttaatgtaatgttaga

SEQ ID NO: 4 - DNA, 20 bp, synthetic construct
  source 1..20: mol_type = other DNA
cccccccccccccccccccc

SEQ ID NO: 5 - DNA, 19 bp, synthetic construct
  source 1..19: mol_type = other DNA
gtaggtaaaaacctaatat

SEQ ID NO: 6 - DNA, 20 bp, synthetic construct
  source 1..20: mol_type = other DNA
gttcgtttagagaacagatc

SEQ ID NO: 7 - DNA, 21 bp, synthetic construct
  source 1..21: mol_type = other DNA
taaagttcgtttagagaacag

SEQ ID NO: 8 - DNA, 15 bp, synthetic construct
  source 1..15: mol_type = other DNA
agccctgtatacgac

SEQ ID NO: 9 - DNA, 19 bp, synthetic construct
  source 1..19: mol_type = other DNA
gtaggtaaaaacctaatat

SEQ ID NO: 10 - DNA, 20 bp, synthetic construct
  source 1..20: mol_type = other DNA
cgtttagagaacagatctac

SEQ ID NO: 11 - DNA, 20 bp, synthetic construct
  source 1..20: mol_type = other DNA
cattgtagatgtcaaaagcc

SEQ ID NO: 12 - DNA, 21 bp, synthetic construct
  source 1..21: mol_type = other DNA
ctccctcatggtggcagttga

SEQ ID NO: 13 - DNA, 18 bp, synthetic construct
  source 1..18: mol_type = other DNA
cggcatgtctattttgta

SEQ ID NO: 14 - DNA, 20 bp, synthetic construct
  source 1..20: mol_type = other DNA
ggtaggtaaaaacctaatat

SEQ ID NO: 15 - DNA, 14 bp, synthetic construct
  source 1..14: mol_type = other DNA
taaaaacctaatat

SEQ ID NO: 16 - DNA, 14 bp, synthetic construct
  source 1..14: mol_type = other DNA
taaaaacctaatat

SEQ ID NO: 17 - DNA, 14 bp, synthetic construct
  source 1..14: mol_type = other DNA
gtaaaaacctaata

SEQ ID NO: 18 - DNA, 14 bp, synthetic construct
  source 1..14: mol_type = other DNA
gtaaaaacctaata

SEQ ID NO: 19 - DNA, 13 bp, synthetic construct
  source 1..13: mol_type = other DNA
taggtaaaaacct

SEQ ID NO: 20 - DNA, 13 bp, synthetic construct
  source 1..13: mol_type = other DNA
taggtaaaaacct

SEQ ID NO: 21 - DNA, 20 bp, synthetic construct
  source 1..20: mol_type = other DNA
gttcgtttagagaacagatc

SEQ ID NO: 22 - DNA, 14 bp, synthetic construct
  source 1..14: mol_type = other DNA
gtttagagaacaga

SEQ ID NO: 23 - DNA, 14 bp, synthetic construct
  source 1..14: mol_type = other DNA
gtttagagaacaga

SEQ ID NO: 24 - DNA, 13 bp, synthetic construct
  source 1..13: mol_type = other DNA
gtttagagaacag

SEQ ID NO: 25 - DNA, 13 bp, synthetic construct
  source 1..13: mol_type = other DNA
gtttagagaacag

SEQ ID NO: 26 - DNA, 13 bp, synthetic construct
  source 1..13: mol_type = other DNA
gttcgtttagaga

SEQ ID NO: 27 - DNA, 13 bp, synthetic construct
  source 1..13: mol_type = other DNA
gttcgtttagaga

SEQ ID NO: 28 - DNA, 21 bp, synthetic construct
  source 1..21: mol_type = other DNA
taaagttcgtttagagaacag

SEQ ID NO: 29 - DNA, 13 bp, synthetic construct
  source 1..13: mol_type = other DNA
gtttagagaacag

SEQ ID NO: 30 - DNA, 13 bp, synthetic construct
  source 1..13: mol_type = other DNA
gtttagagaacag

SEQ ID NO: 31 - DNA, 13 bp, synthetic construct
  source 1..13: mol_type = other DNA
gttcgtttagaga

SEQ ID NO: 32 - DNA, 13 bp, synthetic construct
  source 1..13: mol_type = other DNA
gttcgtttagaga

SEQ ID NO: 33 - DNA, 13 bp, synthetic construct
  source 1..13: mol_type = other DNA
cgtttagagaaca

SEQ ID NO: 34 - DNA, 13 bp, synthetic construct
  source 1..13: mol_type = other DNA
cgtttagagaaca

SEQ ID NO: 35 - DNA, 13 bp, synthetic construct
  source 1..13: mol_type = other DNA
cgtttagagaaca

SEQ ID NO: 36 - DNA, 13 bp, synthetic construct
  source 1..13: mol_type = other DNA
cgtttagagaaca

SEQ ID NO: 37 - DNA, 15 bp, synthetic construct
  source 1..15: mol_type = other DNA
agccctgtatacgac

SEQ ID NO: 38 - DNA, 13 bp, synthetic construct
  source 1..13: mol_type = other DNA
ccctgtatacgac

SEQ ID NO: 39 - DNA, 13 bp, synthetic construct
  source 1..13: mol_type = other DNA
ccctgtatacgac

SEQ ID NO: 40 - DNA, 13 bp, synthetic construct
  source 1..13: mol_type = other DNA
agccctgtatacg

SEQ ID NO: 41 - DNA, 13 bp, synthetic construct
  source 1..13: mol_type = other DNA
agccctgtatacg

SEQ ID NO: 42 - DNA, 13 bp, synthetic construct
  source 1..13: mol_type = other DNA
gccctgtatacga

SEQ ID NO: 43 - DNA, 13 bp, synthetic construct
  source 1..13: mol_type = other DNA
gccctgtatacga

SEQ ID NO: 44 - DNA, 20 bp, synthetic construct
  source 1..20: mol_type = other DNA
cgtttagagaacagatctac

SEQ ID NO: 45 - DNA, 15 bp, synthetic construct
  source 1..15: mol_type = other DNA
gtttagagaacagat

SEQ ID NO: 46 - DNA, 15 bp, synthetic construct
  source 1..15: mol_type = other DNA
gtttagagaacagat

SEQ ID NO: 47 - DNA, 14 bp, synthetic construct
  source 1..14: mol_type = other DNA
agagaacagatcta

SEQ ID NO: 48 - DNA, 14 bp, synthetic construct
  source 1..14: mol_type = other DNA
agagaacagatcta

SEQ ID NO: 49 - DNA, 13 bp, synthetic construct
  source 1..13: mol_type = other DNA
gtttagagaacag

SEQ ID NO: 50 - DNA, 13 bp, synthetic construct
  source 1..13: mol_type = other DNA
gtttagagaacag

SEQ ID NO: 51 - DNA, 20 bp, synthetic construct
  source 1..20: mol_type = other DNA
cattgtagatgtcaaaagcc

SEQ ID NO: 52 - DNA, 15 bp, synthetic construct
  source 1..15: mol_type = other DNA
tgtagatgtcaaaag

SEQ ID NO: 53 - DNA, 15 bp, synthetic construct
  source 1..15: mol_type = other DNA
tgtagatgtcaaaag

SEQ ID NO: 54 - DNA, 14 bp, synthetic construct
  source 1..14: mol_type = other DNA
agatgtcaaaagcc

SEQ ID NO: 55 - DNA, 14 bp, synthetic construct
  source 1..14: mol_type = other DNA
agatgtcaaaagcc

SEQ ID NO: 56 - DNA, 14 bp, synthetic construct
  source 1..14: mol_type = other DNA
cattgtagatgtca

SEQ ID NO: 57 - DNA, 14 bp, synthetic construct
  source 1..14: mol_type = other DNA
cattgtagatgtca

SEQ ID NO: 58 - DNA, 21 bp, synthetic construct
  source 1..21: mol_type = other DNA
ctccctcatggtggcagttga

SEQ ID NO: 59 - DNA, 20 bp, synthetic construct
  source 1..20: mol_type = other DNA
ggtaggtaaaaacctaatat

SEQ ID NO: 60 - DNA, 20 bp, synthetic construct
  source 1..20: mol_type = other DNA
gttcgtttagagaacagatc

SEQ ID NO: 61 - DNA, 21 bp, synthetic construct
  source 1..21: mol_type = other DNA
taaagttcgtttagagaacag

SEQ ID NO: 62 - DNA, 15 bp, synthetic construct
  source 1..15: mol_type = other DNA
agccctgtagacgac

SEQ ID NO: 63 - DNA, 20 bp, synthetic construct
  source 1..20: mol_type = other DNA
ggtaggtaaaaacctaatat

SEQ ID NO: 64 - DNA, 20 bp, synthetic construct
  source 1..20: mol_type = other DNA
cgtttagagaacagatctac

SEQ ID NO: 65 - DNA, 20 bp, synthetic construct
  source 1..20: mol_type = other DNA
cgtttagagaacagatctac

SEQ ID NO: 66 - DNA, 15 bp, unidentified
  source 1..15: mol_type = other DNA
agccctgtatacgac

SEQ ID NO: 67 - DNA, 20 bp, synthetic construct
  source 1..20: mol_type = other DNA
cattgtagatgtcaaaagcc

SEQ ID NO: 68 - DNA, 21 bp, synthetic construct
  source 1..21: mol_type = other DNA
ctccctcatggtggcagttga

SEQ ID NO: 69 - DNA, 20 bp, synthetic construct
  source 1..20: mol_type = other DNA
ggtaggtaaaaacctaatat

SEQ ID NO: 70 - DNA, 20 bp, synthetic construct
  source 1..20: mol_type = other DNA
gttcgtttagagaacagatc

SEQ ID NO: 71 - DNA, 21 bp, synthetic construct
  source 1..21: mol_type = other DNA
taaagttcgtttagagaacag

SEQ ID NO: 72 - DNA, 15 bp, synthetic construct
  source 1..15: mol_type = other DNA
agccctgtatacgac

SEQ ID NO: 73 - DNA, 21 bp, synthetic construct
  source 1..21: mol_type = other DNA
ctccctcatggtggcagttga

(19) United States
(12) Patent Application Publication
(10) Pub. No.: US 2023/0201290 A1
(43) Pub. Date: Jun. 29, 2023

(54) TGF-BETA INHIBITION, AGENTS AND COMPOSITION THEREFOR

(71) Applicant: GMP BIOTECHNOLOGY LIMITED, Agoura Hills, CA (US)

(72) Inventor: Vuong TRIEU, Agoura Hills, CA (US)

(21) Appl. No.: 17/946,578

(22) Filed: Sep. 16, 2022

Related U.S. Application Data
(63) Continuation-in-part of application No. PCT/IB2021/052293, filed on Mar. 18, 2021.
(60) Provisional application No. 62/991,174, filed on Mar. 18, 2020.
(60) Provisional application No. 62/992,825, filed on Mar. 20, 2020.
(60) Provisional application No. 63/005,535, filed on Apr. 6, 2020.
(60) Provisional application No. 63/005,541, filed on Apr. 6, 2020.
(60) Provisional application No. 63/020,889, filed on May 6, 2020.
(60) Provisional application No. 63/090,353, filed on Oct. 12, 2020.
(60) Provisional application No. 63/138,847, filed on Jan. 19, 2021.

(51) Int. Cl. A61K 36/282; C12N 15/113; A61P 31/14; A61K 31/366; A61K 31/12; A61K 36/324; A61K 31/375; A61K 9/48

(52) U.S. Cl. CPC A61K 36/282; C12N 15/1136; A61P 31/14; A61K 31/366; A61K 31/12; A61K 36/324; A61K 31/375; A61K 9/4866; A61K 9/4858; C12N 2310/11; C12N 2310/321

(57) ABSTRACT

The present disclosure relates to TGF-beta inhibition utilizing certain agents such as Artemisinin and antisense oligonucleotides including OT-101. The present invention also provides the composition comprising the said agents optionally along with one or more additional therapeutic agent, method of treating various viral diseases including COVID-19 and method of use involving said agents. The present invention further provides a substantially pure Artemisinin having a purity of more than 90%. The present invention also provides Artemisinin for use in the treatment of COVID-19. The present invention provides a process of extraction of artemisinin and a composition of matter comprising artemisinin. The present invention also provides a method of treating TGF-beta storm. The present invention also provides a method of use of anti-sense oligonucleotide by suppression of TGF-beta induced proteins including IL-6, TGFBIp.

### SEQUENCE LISTING

This application includes a sequence listing submitted electronically as an ST.26 file created on Dec. 15, 2022, named 018988-002US1.xml, which is 65,063 bytes in size and is hereby incorporated by reference in its entirety.

### BACKGROUND OF THE ART

Transforming growth factor β (TGF-β) is part of a larger superfamily of secreted dimeric multifunctional proteins that also includes activins and bone morphogenetic proteins. TGF-β is a multifunctional set of peptides that controls proliferation, differentiation, and other functions in many cell types. The TGF-β signaling pathway can be activated through the interaction of TGF-β ligand with its cognate type I and type II single-pass transmembrane receptors (i.e., TβRI and TβRII, respectively) that are endowed with intrinsic serine/threonine kinase activity. Three TGF-β isoforms have been identified, TGF-β1, 2, and 3, which share 70% sequence identity, bind the same TGF-β type I and type II receptor complex and activate the same downstream intracellular signaling pathways.

Inhibitors for the TGF-beta (TGF-β) superfamily have great potential for multiple clinical applications. Several molecules can inhibit TGF-beta family members. Development of TGF-beta inhibitors and innovation of new technology for in vivo drug delivery will undoubtedly increase options for therapy against viral diseases and other diseases/disorders in which the TGF-beta family plays a significant role.

Efficient agents are required for inhibition of TGF-beta (TGF-β). This also becomes important in view of epidemics and infections caused by various viruses, bacteria, fungus, etc.

Diseases like SARS, MERS, bird flu, swine flu etc. have affected the humanity to great extent in recent years.

Since late 2019 an outbreak of upper respiratory infection and pneumonia caused by a novel coronavirus, SARS-CoV-2 (also known as COVID-19), has rapidly spread from its epicenter in Wuhan in Hubei province of China to become a global epidemic with millions of cases and thousands of deaths. It is believed the outbreak has a zoonotic origin with animal to human transmission followed by human to human spread via aerosol droplets and contaminated surfaces. As with the prior outbreaks of SARS and MERS, numerous approaches are being taken in an attempt to treat and prevent the disease. The genome information for SARS-CoV-2 is known and has been shared. A reliable assay using real-time reverse transcription polymerase chain reaction (RT-PCR) has been developed and is in widespread use. There are reportedly hundreds of clinical studies in progress in globally targeting the diagnosis and treatment of COVID-19. On the clinicaltrials.gov website there are at least 5000 such clinical studies recorded and the list is growing each day. Most are active and enrolling patients. These trials span the therapeutic spectrum and include some diagnostic studies and some trials evaluating traditional medicine and herbal remedies. Most of the listed studies are assessing existing drugs with some evidence of antiviral activity either as monotherapy or in combination.

The classes of agents include antivirals (protease inhibitors, nucleotide analogues), non-steroidal anti-inflammatory drugs, corticosteroids, immunomodulators, monoclonal antibodies, polyclonal antibody preparations, washed microbiota and umbilical cord mesenchymal stem cells.

Despite numerous studies—no agent was found to be effective except for Dexamethasone. The early therapeutic candidates have starting to yield clinical insights that can help with the development of the next wave of anti-COVID-19 therapies. The three early drug therapeutics include: 1) chloroquine and hydroxychloroquine/azithromycin combination 2) remdesivir and 3) sarilumab.

Lessons learned from Hydroxychloroquine:

Hydroxychloroquine and chloroquine are FDA-approved to treat or prevent malaria. Hydroxychloroquine is also FDA-approved to treat autoimmune conditions such as chronic discoid lupus erythematosus, systemic lupus erythematosus in adults, and rheumatoid arthritis. Although reports have surfaced regarding the use of chloroquine and hydroxychloroquine, drugs used in malaria, the efficacy for COVID-19 remains uncertain at best or not effective and toxic at worse. Hydroxychloroquine has known in vitro activity against the SARS-CoV-2 virus. However, the EC50s for SARS-CoV-2 virus are different than for malaria with a >20-fold difference higher in vitro EC50 of hydroxychloroquine for SARS-CoV-2 vs malaria. EC50 values for SARS-CoV-2 virus in the literature have ranged from 0.72-17.31 μM, with higher EC50 values generally associated with higher multiplicity of infections, indicative of a potential need for greater systemic exposure for the higher viral loads. For the FDA recommended treatment dose of malaria (800 mg loading dose followed by 400 mg daily for a total of 3 days), simulations predicted 89% of subjects would have troughs above the target on day one, however this number dropped to 7% by day 14 post after start of prophylaxis. This suggest that higher dosing is needed where the safety profile may not be as robust as the approved dose for malaria. The current FDA approved dosing for malaria treatment (800 mg followed by 400 mg at 6, 24 and 48 hours after the initial dose, a total of 3 days) and prophylaxis (400 mg weekly) and other regimens such as those tested in recent COVID-19 trials (i.e., 400 mg/day for five days or 200 mg three times daily for six days).

Lessons learned from Remdesivir:

Remdesivir (GS-5734) is an investigational monophosphoramidate prodrug of an adenosine analog that was developed by Gilead Sciences, Inc. in response to the Ebola outbreak in West Africa from 2014 to 2016. In its active triphosphate nucleoside form, remdesivir binds to ribonucleic acid (RNA)-dependent RNA polymerase and acts as an RNA-chain terminator. It displays potent in vitro activity against SARS-CoV-2 with an EC50 at 48 hours of 0.77 μM in Vero E6 cells. NIAID/NIH trial of the drug was double-blinded and placebo-controlled: patients in the treatment group received 200 mg of the drug the first day and 100 mg each day thereafter, for up to ten days. Participants needed to test positive for the virus and have evidence of lung involvement in the disease. The primary endpoint was improved time to recovery (discharge from the hospital or ability to return to normal activity), and remdesivir was statistically better than placebo: 11 days versus 15 days. However, there was no impact on survival (8% mortality in the treatment group, 11.6% in the placebo group). The double-blind, placebo-controlled trial in China with 237 patients enrolled patients that had 12 days or fewer of symptoms, confirmed viral infection, and involvement of the lungs as well (<94% oxygen saturation on room air and confirmed viral pneumonia on X-ray). The dosing of remdesivir was the same as in the NIH trial, but the use of the drug was not associated with a shorter time to clinical improvement. A subgroup analysis showed a trend (not statistically significant) towards shorter duration in the patients who overall showed symptoms for ten days or less, though. Mortality was identical between the two groups, although there was again a trend (not significant) towards less mortality on remdesivir in the shorter-duration patients. The viral load was checked in both the upper and lower respiratory tracts of the patients, and remdesivir had no effect on viral load compared to placebo, in any group. It is possible that the patients were more severe cases of COVID-19 due to lack of bed availability. The data would suggest that remdesivir is more effective early on or as prophylactic than for more severe cases. This maybe true of anti-viral that is targeting only viral replication.

Lessons Learned from Tocilizumab:

Tocilizumab is a humanized monoclonal antibody that inhibits both membrane-bound and soluble interleukin-6 (IL-6) receptors. Interleukin-6, which is secreted by monocytes and macrophages, is one of the main drivers of immunologic response and symptoms in patients with cytokine-release syndrome (CRS). Although tocilizumab was first approved by the FDA in 2010 for the treatment of rheumatoid arthritis, it has gained traction in recent years for treatment of patients with CRS following chimeric antigen receptor T-cell (CAR T) therapy as a corticosteroid-sparing agent. Indeed, it received FDA approval for severe or life-threatening CAR T-associated CRS in 2017 due to its efficacy and safety profile. Hyperinflammatory states and cytokine storming, including elevated IL-6, has been reported in severe COVID-19 and were associated with increased mortality in patients in China. A preprint (nonpeer reviewed) case series of 21 patients treated with tocilizumab between Feb. 5 and 14, 2020 in China reported marked success, including rapid resolution of fever and C-reactive protein, decreased oxygen requirements, and resolution of lung opacities on computerized tomography imaging. The authors state the patients all had “routine treatment for a week” before tocilizumab, which was described as “standard care according to national treatment guidelines” including lopinavir, methylprednisolone, and other supportive care. All patients had IL-6 analyzed before tocilizumab administration with a mean value of 132.38±278.54 pg/mL (normal <7 pg/mL). It now seems that utility lies only in critically ill patients, meaning those requiring mechanical ventilation or high-flow oxygenation. Sanofi and Regneron announced on Apr. 27, 2020 that an ongoing phase II/III trial would continue with these subjects only, after an interim analysis found little benefit in less-severe patients. The data would be consistent with cytokine storm being important only in the late stage of COVID-19 associated ARDS. The suppression of the inflammatory reaction without resolution of the underlying infection may not be optimal for treatment of COVID-19 and therefore a combination of an anti-viral agent would be appropriate.

### Integrated Understanding of Current COVID-19 Landscape:

Clinical experience with COVID-19 disease progression and the therapeutics such as Remdesivir and Tocilizumab would suggest that there are two phases of COVID-19—the immune defense based protective phase and the second inflammation driven damaging phase (FIG. 1). Remdesivir has a role early during the initial infection and Tocilizumab has a role late at the end-stage of the disease. There is nothing available currently to combat COVID-19 in the mid stage during the transition from early infection to the hyperinflammation phase—the pulmonary phase. This pulmonary phase is where we think TGF-β plays the dominant role and where TGF-β inhibitor would have a major impact. The recently demonstrated anti-viral activity of OT-101—like that of Remdesivir—would be supportive of its use at early stage of the infection, possibly as a combination with Remdesivir. Additionally this anti-viral activity of OT-101 would be beneficial when used in combination with Tocilizumab. The simultaneous suppression of the excessive cytokine storm and elimination of the underlying viral infection together would deliver an effective therapy for COVID-19. TGF-β role in neutrophil recruitment and fibrosis would suggest that OT-101 would also be effective in the terminal stage of the infection. In addition to Tocilizumab, fluid-conservative therapy should also be considered for the endstage. Reducing lung vascular hydrostatic pressures decreases lung edema in the setting of increased lung vascular permeability. The clinical importance of this observation was confirmed when the ARDS Network reported in a 1,000-patient randomized clinical trial that a fluid-conservative strategy significantly reduced the average duration of mechanical ventilation by 2.5 days, a difference that was not affected by use of the pulmonary artery catheter. The primary beneficial mechanism can be explained by a favorable effect on Starling forces: lower vascular pressure reduces transvascular fluid filtration, particularly in the presence of increased lung vascular permeability.

### Role of TGF-β in ALI/ARDS

Acute lung injury (ALI), ARDS, and pneumonia are all pathologies characterized by lung edema and alveolar flooding. Pneumonia mortality is typically caused by flooding of the pulmonary alveoli, preventing normal gas exchange and consequent hypoxemia. Airways normally have a critically regulated fluid layer essential for normal gas exchange and removal of foreign particulates from the airway (A). Maintaining this fluid layer in the alveoli depends critically on sodium reabsorption mediated by epithelial sodium channels (ENaCs) and CFTR chloride channels (B). During ALI, sepsis, inflammation or infection, inflammatory cytokines are produced that inhibit ENaC (C). A decrease in ENaC reabsorption allows fluid to accumulate in the alveoli causing alveolar flood in loss of normal gas exchange and consequent hypoxemia (D). With COVID-19 the ARDS progressed to full blown cytokine storm with IL-6 expression and neutrophil infiltration and neutrophil NET and related thrombosis.

Transforming growth factor-β is a pathogenic cytokine, which has been implicated in the early phase of acute lung injury (ALI) prior to ARDS. TGF-β levels were increased in ARDS patients compared to healthy controls. Furthermore, active TGF-β levels were more than doubled in the epithelial lining fluid from ARDS patients. TGF-β also reduces the ability to produce multiple steroids, leading to the inability for self-healing and furthering inflammatory damage, in addition to the activation of multiple Smad pathways. Some of these pathways may be insensitive to corticosteroid treatment.

Other studies have specifically explored the role of TGF-β in alveolar flooding. Using a bleomycin-induced lung injury model, TGF-β-inducible genes were dramatically increased as early as 2 days, suggesting that TGF-β may precede alveolar flooding. Of interest, TGF-β may actually remain latent locally, covalently attached to a latency-associated peptide (LAP); pulmonary epithelial cells can activate and cause dissociation of TGF-β from LAP. One member of the integrin family, αvβ6, was recently shown to be a ligand for LAP. αvβ6 is expressed normally at lower levels, yet increased significantly with injury revealing a novel mechanism for rapid and local TGF-β activation. TGF-β is also redox sensitive, and in vitro models of increased ROS via ionizing radiation revealed another mechanism for TGF-β activation. Together, these studies show multiple, redundant possibilities for systemic and paracrine TGF-β activation during lung injury.

One of the first studies to directly implicate TGF-β in regulating ENaC was by Frank and colleagues. They showed that TGF-β reduced amiloride-sensitive Na+ transport in lung epithelial cells. Additionally, TGF-β reduced aENaC mRNA and protein expression via an ERK1/2 pathway in a model of ALI, thus promoting alveolar edema. In vivo studies then showed that TGF-β reduces vectorial Na+ and water transport and that this process occurs independently from increases in epithelial permeability. Interestingly, TGF-β was also found to have an integral role in ENaC trafficking. Peters and colleagues were the first to demonstrate this acute regulation of ENaC in the lung; they found that TGF-β induces ENaC internalization via interaction with ENaCβ. In summary, TGF-β has been implicated in multiple mechanisms reducing ENaC expression and apical localization, thus contributing to the pathophysiology of ARDS and pulmonary edema.

In summary, TGF-β impaired pulmonary barrier function through: 1) Decreases lung epithelial barrier function, 2) Increases the permeability of pulmonary endothelial monolayers, 3) Increases the permeability of alveolar epithelial monolayers, 4) Disrupts alveolar epithelial barrier function by activation of macrophages, and 5) Induces endothelial barrier dysfunction via Smad2-dependent p38 activation. The local activation of TGF-β is critical for the development of pulmonary edema in ALI and blocking TGF-β or its activation attenuates pulmonary edema. This neutralization can be done e.g., by the administration of a soluble type II TGF-β receptor, which sequesters free TGF-β during lung injury and protected wild-type mice from pulmonary edema induced by bleomycin or Escherichia coli endotoxin.

Likewise, the anti-inflammatory isoflavone Puerarin has been shown to reduce the ARDS associated inflammatory process in the lungs by inhibiting the expression of TGF-β. Vitamin D3 attenuates lung injury in ARDS by inhibition of TGF-β.

### Role of TGF-β in COVID-19

Inhibitor against TGF-β is expected to be active against COVID-19 at two levels: 1) Cellular level—Inhibition of viral replication by direct inhibition of TGF-β, 2) Patient level—inhibition of viral induced pathologies.

### Inhibition of Viral Replication by Direct Inhibition of TGF-β at Cellular Level:

RSV infection induces TGF-β expression resulting in cell cycle arrest in A549 and PHBE cells. Cell cycle arrest was also shown to enhance RSV replication. Cell cycle arrest can be reversed by blocking with TGF-β antibody or by TGF-β receptor signaling inhibitor, suggesting a role of TGF-β in viral-induced cell cycle arrest. Finally, blocking of TGF-β also resulted in significantly reduced viral protein expression and lower virus titer. Porcine reproductive and respiratory syndrome virus (PRRSV) is an enveloped, single-stranded positive-sense RNA virus, belonging to the family Arteriviridae, genus Rodartevirus. This highly variant virus can cause respiratory disease, abortions, and secondary viral and/or bacterial infection of all-aged pigs, resulting in long-term infection and widespread complex disease by inhibiting immune defense of host. PRRSV induces over expression of TGF-β, in order to unbalance immune system, disarm host surveillance and finally benefit viral survival. Inhibition of TGF-β1 by either TGF-β1 or TGF-β2 short interfering RNA (shRNA) inhibits PRRSV replication and improves cell viability when PBMCs were infected with the virus.

Coronavirus entry into cells is followed by suppression of cellular replication and redirection of cellular machineries to the replication of the virus. SARS-CoV infection of VeroE6 cells inhibits cell proliferation by both the phosphatidylinositol 3′-kinase/Akt signaling pathway and by apoptosis. The nucleocapsid protein of SARS-CoV inhibits the cyclin-cyclin-dependent kinase complex and blocks S phase progression in mammalian cells including VeroE6. And, SARS-CoV 7a protein blocks cell cycle progression at G0/G1 phase via the cyclin D3/pRB pathway of HEK293, COS-7, and Vero cells. Murine coronavirus replication induces cell cycle arrest in G0/G1 phase in infected 17Cl-1 cells through reduction in Cdk activities and pRb phosphorylation. Infection of asynchronous replicating and synchronized replicating cells with the avian coronavirus infectious bronchitis virus (IBV) arrests infected cells in the G1/M phase of the cell cycle.

Therefore, we hypothesized that TGF-β inhibitors would affect cell cycle regulation following SARS-CoV and SARS-CoV-2 infections, resulting in inhibition of viral replication. We tested various TGF-β inhibitors in the viral replication assay for SARS-CoV-2 (the COVID-19 virus). Indeed, these inhibitors exhibited effective inhibition of SARS-CoV-2. It was found that TGF-β and/or TGF-β pathway was shown to be upregulated during SARS-CoV-2 infection and inhibitor of TGF-β pathway was effective at inhibiting viral replication. This provides pharmacological validation in addition to genetic evidence that TGF-β pathway is critical to SARS-CoV-2 infection in vitro.

a. TGF-β Recruits Neutrophils into the Site of Inflammation Increasing the Risk for Pulmonary Thrombosis

TGF-β is a multifunctional cytokine, playing an important role in the pathology of respiratory viral infection including neutrophil recruitment responsible for the inflammation and pulmonary fluid accumulation that often result in pneumonia and death. Low level of local TGF-βs induces neutrophil chemotaxis to damaged tissue i.e. the lung. TGF-β has the highest migratory distance of the peripheral blood neutrophils (PMNs). As inflammation unfold, high level of TGF-β could contribute to viral pathogenesis through both local phenotypic effects and secondary effects including changes in vascular permeability, resulting from the induction of VEGF or other TGF-β regulated cytokines, chemokines, and growth factors as was shown for Ebola.

The hallmark of acute infection is leukocytosis and neutrophila, which has been seen also in COVID-19. COVID-19 patients suffer from dysregulation of immune response including neutrophilia and lymphophenia resulting in increase in neutrophil lymphocyte ratio (NLR). High NLR is the most significant prognostic predictor of severe illness incidence when 26 variables were included in the LASSO regression. High NLR is more commonly seen in severe patients. High NLR is predictive of worse outcome including lower recovery rate, higher mechanical ventilation rate, higher need for oxygen therapy. High NLR is expected to have increase neutrophil/macrophage derived cytokine release storm (CRS) and aggravation by virus associated tissue damage and complication. Neutrophilia alone also predicts poor outcomes in patients with COVID-19. Neutrophil infiltration in pulmonary capillaries, acute capillaritis with fibrin deposition, extravasation of neutrophils into the alveolar space, and neutrophilic mucositis have been reported for COVID-19. These findings are consistent with elevated TGF-β serving as neutrophil chemotaxis into COVID-19 affected lungs resulting pulmonary thrombosis. The elevated TGF-β expression among RNAs isolated from the bronchoalveolar lavage (BAL) fluid of COVID-19 patients versus normal controls has been reported and is supportive of this concept.

b. TGF-β Inhibits ENaC and Causes Fluid Accumulation in the Lung and ARDS/Pneumonia

TGF-β has been implicated as an important pro-inflammatory cytokine in the pathophysiology of acute lung injury and Acute respiratory distress syndrome (ARDS) that contributes to both increased permeability and failed fluid reabsorption in lungs leading to persistent and severe pulmonary edema. Importantly, bronchoalveolar lavage fluid (BAL) samples from ARDS patients collected within 2 days after intubation showed higher TGF-β levels when compared to BAL samples from non-ARDS controls. TGF-β can increase alveolar epithelial permeability and pulmonary endothelial permeability by promoting adherens junction disassembly as well as inhibiting pulmonary endothelial proliferation. SARS-CoV has been shown to up-regulate pro-inflammatory cytokines, including TGF-β and TGF-β levels were markedly elevated in SARS patients with ARDS.

A recent study by Peters et al. demonstrated that TGF-β profoundly impacts alveolar ion and fluid transport by regulating the epithelial sodium channel (ENaC) activity and trafficking via a Tgfbr1-mediated unique signalling pathway. TGF-β was identified as the exclusive master regulator of ENaC internalization by alveolar epithelial cells and its upregulation in ARDS causes an ENaC trafficking defect with marked reduction in the cell-surface abundance of ENaC on lung epithelial cells thereby rapidly and substantially impairing alveolar fluid reabsorption in ARDS patients and contributing to the persistence of their pulmonary edema. A soluble recombinant TGF-β receptor protein capable of sequestering TGF-β has effectively attenuated the severity of pulmonary edema in experimental models of ARDS. Likewise, the anti-inflammatory isoflavone Puerarin has been shown to reduce the ARDS-associated inflammatory process in the lungs by inhibiting the expression of TGF-β. Notably, a significant negative correlation existed between TGF-β levels in BAL samples from ARDS patients and ventilator-free days and ICU-free days. Furthermore, lower TGF-β levels correlated with better survival outcome indicating that that patients with higher TGF-β levels may have a higher and faster case mortality. The correlation of lower BAL fluid TGF-β levels with improved survival of ARDS patients further supports the concept of reducing TGF-β levels with Artemisinin and/or OT-101 in patients with ARDS. OT-101 is also known as Trabedersen and are used interchangeably throughout here.

c. TGF-β Induces Late Stage Fibrosis Compromising Lung Capacity Even After Recovery

TGF-β is also involved in the pathogenesis of lung tissue remodeling and lung fibrosis that follows ARDS. Specifically, TGF-β contributes to the development of lung fibrosis by stimulating the proliferation/differentiation of lung fibroblasts, accumulation of collagen and other extracellular matrix proteins in the pulmonary interstitial and alveolar space, leading to the occurrence and development of pulmonary fibrosis. Wang et al. reported that miR-425 reduction in lung fibroblasts contributes to the development of lung fibrosis post ARDS through activation of the TGF-β signalling pathway. Smad2, a key component of the canonical TGF-β signaling pathway was discovered to be regulated by miR-425. Therefore, inhibition of the TGF-β signaling pathway also has the potential to prevent development of pulmonary fibrosis following ARDS and improve the pulmonary healing process.

d. TGF-β Induces IL-6 Leading to Systemic Inflammation and “Cytokine Storm”

Treatment with only TGF-β, leads to the induction of IL-6, and this was both dose- and time-dependent. The effect of TGF-β was evident at the level of IL-6 mRNA, suggesting TGF-β induced de novo synthesis of IL-6. The ability of TGF-β, to induce IL-6 suggests that IL-6 may mediate some of the effects of TGF-β. Elevated expression of the immunomodulatory and fibrogenic cytokine TGF-β is evident in the airway smooth muscle cells (ASMCs) of asthmatic and chronic obstructive pulmonary disease (COPD) patients. An important inflammatory effect of TGF-β on ASMCs is the induction of IL-6 release. TGF-β plays a pivotal role in driving ASMCs toward a prooxidant and proinflammatory phenotype. TGF-β disrupts oxidant/antioxidant balance and increases IL-6 release in ASMCs, through Smad and PI3K-dependent pathways. TGF-β1 also promotes the release of IL-6 in BAL cells. TGF-β1 primed the mast cell for IL-6 production upon stimulation, rather than drove IL-6 production directly. In doing so TGF-β drives the innate inflammation in the lung.

However, it must be emphasized that median IL-6 levels in patients with the hyperinflammatory phenotype of ARDS/CRS are 10- to 200-fold higher than those in patients with severe COVID-19. Notably, the peak plasma IL-6 level in patients who developed CRS was approximately 10,000 pg/mL—almost 1000-fold higher than that reported in severe COVID-19. Therefore, IL-6 induced cytokine storm is not the main cause for the life-threatening ARDS and coagulopathy observed in severe COVID-19 patients. Instead, it is the IL-6 upregulation by TGF-α that is important in COVID-19. It would be ineffective to focus on the subcomponents (IL-6) instead of the main pathology (TGF-β) as demonstrated by the failure of two major phase 3 studies using mAb against IL-6 receptor in COVID-19. Kevzara® (sarilumab), a human monoclonal antibody against the IL-6 receptor, failed in Phase 3 trial conducted by Sanofi and Regeneron Pharmaceutical, Inc. Tocilizumab failed its phase III COVACTA study. The difference in clinical status between Actemra/RoActemra and placebo in patients assessed using a 7-category ordinal scale at week four was not statistically significant (p=0.36; odds ratio [95% CI]=1.19 [0.81, 1.76]).

e. TGF-β Induces TGFBIp Leading to Vascular Inflammation

Transforming growth factor—induced protein (TGFBIp), an extracellular matrix protein functionally associated with the adhesion, migration, proliferation, and differentiation of various cells, has been identified and cloned as a major TGF-β-responsive gene in the lung adenocarcinoma cell line A549: TGF-β-induced gene human clone 3, abbreviated to βig-h3. TGFBIp is secreted from several cell lines, including epithelial cells, endothelial cells, keratinocytes, fibroblasts, and monocytes and exists in the ECM. It is a 68-kDa protein that consists of 4 fasciclin 1 domains (FAS1 domain) and a carboxy-terminal arginyl-glycyl-aspartic acid (RGD) sequence, both of which potentially bind integrins as a cell attachment site.

It found that TGFBIp and its derivative TGFBIp K676Ac, acetylated 676th lysine TGFBIp, are elevated in the blood of SARS-CoV-2 pneumonia patients (n=113); especially in intensive care unit (ICU) patients than non-ICU patients. Under inflammatory conditions, the secreted TGFBIp becomes acetylated by CBP/p300, then the RGD domain of the secreted TGFBIp K676Ac binds with integrin αvβ5 and activates NF-κB to induce inflammation. TGFBIp promotes severe vascular inflammatory responses and thrombogenesis. Its neutralization using anti-TGFBIp antibody significantly reduced the secretion of proinflammatory cytokines by PBMC of SARS-CoV-2 patients. The observed increase plasma level of TGF-β level among severe COVID-19 patients would support this concept that TGF-β is inducing inflammatory/vasculitis factors resulting in the multitude of COVID-19 pathologies.

f. TGF-β Induces IgA Class Switching Leading to IgA Vasculitis/Kawasaki Disease Syndrome

Kawasaki Disease (KD) is an IgA vasculitis disease arisen from the class switching of the initial IgM to IgA. TGF-β is known to induce switching to IgA antibody production in B cells in combination with IL-10. It has been shown that in Epstein-Barr virus (EBV)-associated diseases there is a positive correlation between EBV-specific IgA titers and the levels of TGF-β. Similar to KD, IgA is upregulated in COVID-19 patients and is one of its hallmark characteristics. Enhanced IgA responses observed in severe COVID-19 might confer damaging effects in severe COVID-19. As a result, it was hypothesized that severe COVID-19 might be at least in part an IgA mediated disease (related to IgA deposition and vasculitis), which helps to explain common organ injuries in COVID-19, e.g. acute pulmonary embolism, kidney injury, etc.

It has been shown in the art that the first seroconversion day of IgA was 2 days after onset of initial symptoms, and the first seroconversion day of IgM and IgG was 5 days after onset. The relative levels of IgA and IgG were markedly higher in severe patients compared to non-severe patients. There were significant differences in the relative levels of IgA (P<0.001) and IgG (P<0.001) between the severe and non-severe groups. In contrast, no statistically significant changes occurred in the levels of IgM between severe and non-severe patients after the disease onset. In further subgroup analysis, it was found a significant positive association of SARS-CoV-2 specific IgA level and the APACHE II score in critically ill patients with COVID 19 (r=0.72, P=0.01), while the level of SARS-CoV-2 specific IgG and IgM did not show correlations with disease severity.

The cardiovascular symptoms in COVID-19 could be related to IgA vasculitis as increased IgA relative to IgG was observed among severe COVID-19 patients. Although there is currently no evidence for antibody dependent enhancement in COVID-19 this would be consistent with the Kawasaki syndromes and consistent with previously observed antibody dependent enhancement of SARS.

Global autoantibody screening against SARS-CoV-2 antigens found strong enrichment in MIS-C of autoantibodies to proteins involved in immune response regulation and structural proteins in the heart, in particular the glycoprotein Endoglin. Endoglin (CD105), also called TGF-β receptor III is a homodimeric membrane protein that binds TGF-β. Taken together, IgA vasculitis and enrichment of endoglin in heart muscle and lung can result in the immune reactions occurred at these sites which could further explain the common organ injuries in COVID-19. Therefore, we believe that TGF-β inhibition can reduce IgA vasculitis (KD syndrome) and will improve COVID-19.

### COVID-19 —a TGF-β Storm Disease:

Coronavirus entry into cells is followed by suppression of cellular replication and redirection of cellular machineries to the replication of the virus. SARS-CoV-1 infection of VeroE6 cells inhibits cell proliferation by both the phosphatidylinositol 3′-kinase/Akt signaling pathway and by apoptosis. The nucleocapsid protein of SARS-CoV-1 inhibits the cyclin-cyclindependent kinase complex and blocks S phase progression in mammalian cells including VeroE6. And, SARS-CoV-1 7a protein blocks cell cycle progression at G0/G1 phase via the cyclin D3/pRB pathway of HEK293, COS-7, and Vero cells. Murine coronavirus replication induces cell cycle arrest in G0/G1 phase in infected 17Cl-1 cells through reduction in Cdk activities and pRb phosphorylation. Infection of asynchronous replicating and synchronized replicating cells with the avian coronavirus infectious bronchitis virus (IBV) arrests infected cells in the G1/M phase of the cell cycle. Cell cycle arrest is also centrally mediated by up-regulation of TGF-β. SARS coronavirus upregulates TGF-β via its nucleocapsid protein and papain-like protease (PLpro). SARS coronavirus PLproactivates TGF-β1 transcription both in cell-based assay and in mouse model with direct pulmonary injection. TGF-β overexpression in SARS patients lung samples also been demonstrated. Suppression of TGF-β expression by OT-101 suppressed SARS-CoV1 and SARS-CoV2 replication in the viral replication assays [OT-101 Investigator Brochure, University of Utah report. In the same study, artemisinin—a reported TGF-β inhibitor—also suppressed SARS-CoV2 replication. Therefore, it is most likely that induction of TGF-β following infection results in cell cycle arrest to allow for diversion of cellular machinery to viral production. This means as viral load increases there will be a proportional increase in TGF-β which in turn drives the progression of COVID-19 disease. It has been reported that viral load is closely associated with drastically elevated IL-6 level in critically ill COVID-19 pts and mortality. By targeting TGF-β, OT-101 shuts off the engine behind COVID-19 allowing patients to recover without going into respiratory crisis. In fact, the administration of a soluble type II TGF-β receptor, which sequesters free TGF-β during lung injury and protected wild-type mice from pulmonary edema induced by bleomycin or Escherichia coli endotoxin. The local increase in TGF-β can also trigger a cascading event leading to recruitment of neutrophil, NET to the infected organ and the resulting coagulation releases TGF-β stored in platelets with precipitous consequences.

The architecture of the alveolus, which is composed of type I and type II alveolar epithelial cells, resident intra-alveolar macrophages and adjacent lung capillaries with intact endothelial lining. The injured alveolus in ALI/ARDS: Complement activation products (C5a) and inflammatory mediators released by activated macrophages orchestrate the influx of PMNs, monocytes and adaptive immune cells to the alveolar compartment. C5a promotes release of NETs and extracellular histones, thereby resulting in tissue damage and disruption of the epithelial/endothelial barrier. Intraalveolar hemorrhage includes the presence of platelets, which interact with NETs and release TGF-β. The later phases of ALI/ARDS may include TGFβ-mediated fibro-proliferative responses and accumulation of extracellularmatrix. For COVID-19, the triggering event is not injury to the organ but the release of TGF-β generated during viral replication and infection.

In the case of SARS-CoV infection, the serum level of TGF-β1 was elevated during the early phase of SARS (Pang et al. 2003). A high level of TGF-β was also observed in SARSCoV-infected lung cells (including alveolar epithelial cells, bronchial epithelial cells, and monocytes/macrophages), but not in uninfected lung cells (Baas et al. 2006; He et al. 2006). The virus-induced high level of serum and in situ TGF-β ligand leads to hyperactivation of the TGF-β pathway leading to SARS/ARDS.

### OT-101 (Inhibitor of TGF-β) for COVID-19:

There is an emerging recognition that TGF-β could be a valid target for the treatment of COVID-19. Alhelfawi M. et al suggested that COVID-19 can be treated with TGF-β inhibition. SARS-CoV PLpro significantly induced the TGF-β-mediated pro-fibrotic response via ROS/p38 MAPK/STAT3/Egr-1 pathway in vitro and in vivo. PLpro also triggered Egr-1 dependent transcription of TSP-1 as an important role in latent TGF-β1 activation. Blocking TGF-β would inhibit or reduce the complication of viral spread and fibrosis as well as giving chance for cellular immunity to exert its effect and hence reduction of the viral yield in the infected cells. In fact, knockdown of TGF-β gene expression by shRNA not only inhibits the replication of PRRSV but also improves immune responsiveness following viral infection, suggesting a novel way to facilitate the control of PRRSV infection in pigs. Epithelial cells have a vital role orchestrating pulmonary homeostasis and defense against pathogens. TGF-β regulates an array of immune responses—both inflammatory and regulatory—however, its function is highly location—and context-dependent. Epithelial derived TGF-β acts as a pro-viral factor suppressing early immune responses during influenza A infection. Mice specifically lacking bronchial epithelial TGF-b1 (epTGFbKO) displayed marked protection from influenza-induced weight loss, airway inflammation, and pathology.

However, protection from influenza-induced pathology was not associated with a heightened lymphocytic immune response. In contrast, the kinetics of interferon beta (IFNb) release into the airways was significantly enhanced in epTGFbKO mice compared with control mice, with elevated IFNb on day 1 in epTGFbKO compared with control mice. This induced a heighted antiviral state resulting in impaired viral replication in epTGFbKO mice. Their publication succinctly described the impact of TGF-β suppression against viral infection and we would propose that OT-101 would result in very similar if not the same responses—with the exception that the TGF-β storm driven by viral infection of epithelial cells and multiplication as the source of TGF-β.

As a result, the sudden and uncontrolled increases in active TGF-β (possibly with the help of some proinflammatory cytokines such as TNFα, IL-6, and IL-1β) inevitably result in rapid and massive edema and fibrosis that remodels and ultimately blocks the airways. This leads to the functional failure of the lungs and death of the patients.

Summation of the data we would like to propose the progression of COVID-19 and the placement of OT-101 versus Remdesivir and others in the treatment of COVID-19 as below.

TABLE 1
The three phases of COVID-19 and the application
of OT-101, Vegetarian, and energy

−αENaC mRNA and
protein expression

Clinical Impacts of TGF-β Storm

| | | |
|---|---|---|
| Normal Fluid | Alveolar edema | Alveolar edema |
| Clearance | | Cytokine storm |
| | | Neutrophil NET |

OT-10 Mechanism of Action

| | | |
|---|---|---|
| Viral | Viral replication ↓ | Viral replication ↓ |
| replication ↓ | Alveolar edema ↓ | Alveolar edema ↓ |
| | | Neutrophil NET ↓ |

Positioning of Therapeutic Agents

| | | |
|---|---|---|
| Antiviral Agent: | Antiviral Agent: | Antiviral Agent: |
| Remdesivir or | Remdesivir or OT-101 | Remdesivir or OT-101 |
| OT-101 | Inhibitor of TGF-β | Inhibitor of TGF-β |
| | Storm: OT-101 | Storm: OT-101 |
| | | Inhibitor of Cytokine |
| | | Storm: Tocilizumab |
| | | (anti-IL-6) Fluid- |
| | | conservative strategy |

OT-101 and cytokine Risk:

Despite extensive data-mining, there was no signal of cytokine storm or emergent of cytokine storm following treatment with OT-101. IL-6 was examined and as shown above, does not exhibit meaningful changes across time following treatment with OT-101. We do not anticipate treatment of COVID-19 patients with OT-101 will result in emergent of cytokine storm. However, due to this potential risk, the protocol is designed with a lead in phase where patients will be entered in a staggered fashion with a minimum of 48 hours between consecutive patients. Patients will be monitored for AE and several assessments will be done to confirm the safety of OT-101 in this patient population. Patients will be monitored for AEs continually until day 14. Safety data will be reviewed on day 7 and day 14 after the initiation of treatment in the first patient by the Data Safety Committee (DSC). It is anticipated that safety data up to day 7 will be available on all 7 patients by day 14 and reviewed by DSC. Part 2—the expansion part of the trial—will be initiated only if OT-101 is not associated with drugrelated SAE in Part 1 in any of the 7 patients. Cytokine profiling was performed during P001 and the analysis reported in two reports: 1) Analysis of plasma levels of 31 cyto-/-chemokines in selected samples of study P001, Study No: AP 12009/021301 and 2) Analysis on Cytokine Response in Patients Treated with OT-101 (Trabedersen).

Thirty-one cyto/chemokines from clinical plasma samples of 12 pancreatic cancer patients of the P001 study were assessed in an exploratory analytical study to analyze the impact of OT-101 treatment on cyto/chemokine levels in plasma. Samples analyzed were acquired from before onset of OT-101 therapy during the screening phase of the study and at selected time points during the therapy. Samples were measured and data were acquired by Lophius-Biosciences applying a non-validated method allowing the analysis of multiple cytokines. Regression and hierarchical cluster analyses were performed to identify potential cytokine signatures in the patients investigated in this cohort. Analysis of variance models were applied to investigate relationships between cyto/chemokine levels and clinical outcomes (PK and OS). Logistic models were applied to characterize associations of cyto/chemokine levels and adverse events.

Transforming growth factor-beta (TGF-β) is a multifunctional regulatory polypeptide that controls many aspects of cell function—including cell proliferation, differentiation, migration, apoptosis, adhesion, angiogenesis, immune surveillance, and survival. TGF-β has a dual role in cancer. It is tumor suppressive in premalignant cells and in the early stage of tumor development, but strongly protumorigenic at later stages of tumor progression. Autocrine TGF-β signaling promotes epithelial—mesenchymal transition, which increases cell invasion and metastasis. Paracrine TGF-β signaling stimulates angiogenesis and contributes to an immune-tolerant environment by suppressing T lymphocytes and natural killer (NK) cells. The overall balance of tumor inflammatory mechanisms is polarized to promote angiogenesis, tumor cell survival, and immune escape, all contributing to tumor growth.

After infection with SARS-CoV-2, up to one third of COVID-19 patients develop an acute pulmonary inflammation with a fulminant progression to acute respiratory distress syndrome (ARDS) with a high fatality rate in high risk patient populations despite best available supportive care. In these patients, it was thought initially that the “burst release” of proinflammatory cytokines in massive amounts and in succession causes a severe form of systemic capillary leak syndrome with pulmonary edema, that can cause hypoxic injury and dysfunction of multiple organs, ultimately leading to an irreversible and fatal multi-organ failure. This hyperacute inflammatory process is reminiscent of the cytokine release syndrome (CRS) that may hyperactivate elements of both the innate and acquired immune system. However, with the failure of IL-6 mAb, it is likely that the cytokine storm was not the primary pathology of COVID-19. Instead, coupling of our finding that ARTIVeda™ has potent antiviral activity and existing knowledge of TGF-β, it has been proposed that TGF-β is the main driver of COVID-19.

TGF-β is upregulated following SARS-CoV-2 infection inducing cell cycle arrest and allowing the virus to hijack the host machineries for its own replication. The increase in viral load results in a TGF-β surge causing a diverse clinical symptoms associated with COVID-19. Instead of targeting the individual subcomponents i.e. mAb against IL-6, it is more effective to target the underlying pathology of these clinical symptoms i.e. inhibition of the TGF-β surge/storm with Artemisinin and/or OT-101 (In April 1975, after the nationwide collaborative conference on artemisinin as an antimalarial, research units from the Academy of Traditional Chinese Medicine now the China Academy of Chinese Medical Sciences (CACMS), Shandong, Yunnan, Guangdong, Sichuan, Jiangsu, Hubei, Henan, Guangxi, Shanghai, the Chinese Academy of Sciences (CAS), and the People's Liberation Army (PLA) formed the China Collaborative Research Group on Qinghaosu (artemisinin). Artemisinin formulations, including suppositories, were used to treat malaria in Hainan, Yunnan, Sichuan, Shandong, Henan, Jiangsu, Hubei, and Southeast Asia.

A total of 1511 cases of vivax malaria were treated with artemisinin. Average defervescence time was 20-30 hours, average asexual parasite clearance time was 30-40 h, and the relapse rate was 10%-30% within a month. The collaborative research group used artemisinin tablets (total dose 3 g) to treat 16 cases of vivax malaria in 1975. Another 13 control cases were given chloroquine, total dose 1.5 g (base). The parasite clearance time of the artemisinin group was 39.6±13.5 h, while that of the chloroquine group was 55.9±16.6 h (P<0.01), indicating that the clearance speed of artemisinin was faster than that of chloroquine. However, the relapse rate of the artemisinin group was 21.4% within a month, while no cases of relapse were seen in the chloroquine group Similar results were observed in other locations.

Artemisinin was used to treat 527 cases of falciparum malaria in various locations. Fever clearance time was 30-40 h, and asexual parasite clearance time was 30-50 h. The recrudescence rate was 85% within a month for tablets, and 10%-25% for other formulations.

Also in 1975, the collaborative research group used 3-day regimens of artemisinin tablets (total dose 2.5 g) and chloroquine (total dose 1.5 g base) to treat 18 cases of falciparum malaria with each drug. Average asexual parasite clearance time was 37±17.8 h for artemisinin and 65.7±29.9 h for chloroquine (P<0.01). Therefore, the clearance speed was faster for artemisinin than for chloroquine. All the cases in the artemisinin group experienced recrudescence in a month, whereas the recrudescence rate for chloroquine was 50%. Although the recrudescence rate for artemisinin was higher than that of chloroquine, the rate dropped to 10%-25% within 1 month when the tablet formulation was replaced by the oil, oil-suspension, and water-suspension formulations.

A total of 143 cases which had failed to respond to chloroquine (RI-RIII resistance) were studied in areas where chloroquine-resistant falciparum malaria was prevalent. The 3-day regimen of artemisinin oil, oil suspension, or water suspension was used and all patients were cured. Therefore, artemisinin was shown to be effective in treating chloroquine-resistant falciparum malaria.

From 1974 to 1978, 141 cases of cerebral malaria were studied in areas where chloroquine-resistant falciparum malaria was endemic. Since injections were not available at the time, nasogastric gavage was used with 36 patients. The cure rate was 91.7%. Intramuscular administration was adopted once injections became available. Of the 141 patients, 131 were cured (92.9%). Ten died, yielding a mortality rate of 7.1%. From statistics across all sites, the average parasite clearance time was 33.3-64.5 h and average defervescence time was 34.1-56.7 h. The average time taken to regain consciousness was 21.5-30.8 h. This did not include a few cases who only regained consciousness after at least 10 days.

No obvious side effects were seen in 2099 cases treated with artemisinin. Tests for liver function and recordings of electrocardiograms (ECGs) were conducted on 139 cases before and after treatment, and 75 cases were examined for nonprotein nitrogen in the blood. No abnormalities were found. There were no aberrations in patients with heart, liver, or kidney diseases, and in pregnant women. For the water suspension, mild pain was experienced at the injection site, but no other adverse reactions were reported.

A case series of hepatotoxicity associated with an extract of Artemisia annua L. was identified through the New Zealand spontaneous adverse drug reaction reporting system. A. annua extract, produced using a supercritical carbon dioxide extraction method and formulated with grapeseed oil, has been marketed in New Zealand as a natural product for joint health. As of 31 Jan. 2019, the New Zealand Pharmacovigilance Centre had received 29 reports of hepatic adverse reactions occurring in patients taking A. annua extract in grapeseed oil. The case reports were assessed for patient and adverse reaction characteristics, patterns of A. annua extract use and causality (based on the WHO-UMC system for standardized case causality assessment). Patients were aged 47 to 93 years (median 67). Time to onset of hepatotoxicity from starting A. annua extract was 7 days to approximately 12 months in the 23 reports with this information. Nineteen of these reports indicated onset within 12 weeks. A. annua extract was the sole suspect medicine in 27 reports. A few patients had possible predisposing conditions. Twenty-seven patients were reported to have recovered or improved on stopping A. annua extract. Nine patients required hospital admission. The pattern of hepatic injury varied. Jaundice, often with pruritus and dark urine, was experienced by 16 patients. There was considerable consistency across case reports from various reporters.

Twenty-three reports described “hepatic enzymes increased”/“hepatic function abnormal.” Five patients experienced “hepatitis,” and one patient experienced “hepatic cirrhosis.” “Jaundice,” often presenting early, was also reported for 16 patients, including seven with “pruritus.” Two reports were suggestive of impaired hepatic synthetic function: one patient had hypoalbuminemia and another experienced purpura and hematuria.

None of the reports described death as an outcome. However, nine patients required hospital admission. In 16 reports, the hepatic adverse effects were described by the reporter as “severe.” Liver function test results were available in 21 reports. Peak concentrations of serum bilirubin, ALP, and ALT were reported for 18, 19, and 21 patients, respectively. Serum bilirubin ranged from 5 to 608 μg/L, (mean 115.3); ALP 73 to 594 IU/L (mean 307.5); and ALT 37 to 3,311 IU/L (mean 517.6).

A. annua has a long history of traditional use, yet there are very few published reports of hepatotoxicity. There are, however, numerous reasons why adverse reactions following use of herbal medicines may not be identified or reported. Typically, individuals who use herbal medicines do not seek professional advice if they experience adverse effects, and use of herbal medicines is often not disclosed to healthcare professionals. The lack of adverse reaction reports for these substances should not be interpreted as evidence of “safety.”

The WHO Global Database of Individual Case Safety Reports, VigiBase®, contains two recent non-New Zealand reports of hepatobiliary disorders associated with A. annua. The first report was confounded by the use of several herbal preparations but the second concerned a male who took A. annua 1.25 g daily for six weeks. He developed cholestatic icterus eight weeks after initiation, which was reported as serious (caused or prolonged hospitalization). The reaction abated after stopping A. annua and the patient recovered.

Artemisinins have been available in the United States without a prescription as herbal supplements for at least 20 years; these supplements are marketed for general health maintenance and for treatment of parasitic infections and cancers. In the CAERS database (2004-2019), there were only 5 cases of toxicity associated with Artemisinin. On Aug. 27, 2008, CDC was notified of a patient who developed hepatitis after a 1-week course of an herbal supplement containing artemisinin. The 52-year old man developed hepatitis after taking artemisinin 200 mg three times daily for one week. Clinical investigation did not reveal any other cause for the hepatitis. He recovered two weeks after stopping artemisinin. The patient had abdominal pain, dark urine, and laboratory results consistent with hepatitis (e.g., serum alanine aminotransferase of 898 IU/L [normal: 10-55 IU/L]). Samples of the supplement were sent to CDC and the Georgia Institute of Technology for analysis to determine the amount of artemisinin and to identify any contaminants. Analysis indicated that the supplement contained 94%-97% of the 100 mg of artemisinin stated on the packaging and the supplement contained no other common pharmaceutical active ingredients. Given the patient's clinical course and laboratory evaluation, CDC investigators concluded that the hepatitis might have been associated with ingestion of the herbal supplement containing artemisinin. A 43-year-old woman developed arthralgia and jaundice five weeks after she began taking artemisinin 125 mg orally two-to-three times daily. A thorough clinical investigation did not reveal any structural, viral, or autoimmune cause for the hepatitis, and paracetamol concentrations were undetectable. Discontinuation of artemisinin resulted in gradual clinical and biochemical improvement, and the woman remained asymptomatic with normal liver function one year later.

The databases of the FDA's Adverse Event Reporting System (AERS) and its Center for Food Safety and Applied Nutrition (CFSAN) Adverse Event Reporting System (CAERS) hold an additional eight reports of hepatotoxicity in patients using artemisinin as self-treatment. An expert review of these case reports and the two published reports noted that artemisinin was used for a longer duration and at a greater dose than for malaria treatment (typically 3 days) and suggested that this may have contributed to hepatotoxicity.

Taken together they do raise a hypothesis for hepatotoxicity with unpurified A. annua extract. Hepatotoxicity associated with the A. annua extract Arthrem® was reported in a randomized, double blind, placebo-controlled pilot study. Of the 28 subjects randomized to Arthrem®, one of 14 participants receiving high-dose Arthrem® (300 mg twice daily) developed hepatitis, considered possibly related to the study medicine by the investigator. Thirty-four participants continued into an open-label, six-month safety extension study of Arthrem 150 mg twice daily. One patient withdrew because of elevated serum hepatic enzymes, considered unrelated to the study medicine by the investigator.

In addition to artemisinin, A. annua extracts contain variable amounts of other constituents, including a series of arteannuins, artemisitine, artemisinic acid, flavonoids, including artemetin, and a volatile oil. The pharmacological activities of many of these compounds are not fully understood. The quantity of particular constituents in the raw herbal material is influenced by numerous factors, such as the growing location and conditions, and the time of harvest. The concentration of artemisinin is highest in the leaves just before the plant flowers. Further, the chemical composition of extracts prepared from A. annua leaves differs depending on the extraction method used.

Arthrem® and Go-Arthri® are prepared from A. annua by supercritical CO2 extraction of dried plant material. This method relies on the fact that carbon dioxide behaves as a liquid when under high pressure and is highly effective for extracting biomass. The seeds were sourced from Switzerland and then grown at high altitudes in Tanzania where the soil is fertile and dense. It takes nine months to fully grow the plants. The nutrient rich tips of the plant were hand picked and dried using traditional methods. After drying, the plants were shipped to NZ where the active compounds were extracted. The natural extract was sent to a Swiss laboratory for analysis. The extract was then combined with grape seed oil to produce the easy-to-swallow soft gel capsule and marketed as Arthrem® and Go-Arthri®.

Safety information on artemisinin is largely obtained from their use as anti-malarial therapy (typically used in conjunction with other drugs), where it is generally considered safe and well-tolerated when administered for several days. Of note however, is the observed liver toxicity of herbal extract containing artemisinin. It is our conclusion that artemisinin as a purified product should only be used and not Artemisinin as herbal extract in regard to clinical safety.

### Artemisinin-Naphthoquine Combination (ARCO®)

ARCO® is a new generation ACT developed by the Chinese Academy of Military Medical Sciences (AMMS) in the early 1990s. It is a product of the combination of two independently developed antimalarials, artemisinin and naphthoquine. The main disadvantages of artemisinin and naphthoquine as monotherapy for malaria infections have been, for artemisinin, a very short circulating half-life as a result of rapid elimination, such that effective concentration levels might not be sustained to ensure complete elimination of blood parasites over several asexual cycles. For naphthoquine, the main disadvantage has been the slowness in the onset of the parasiticidal action following therapy administration. The slowness in the onset of action would create a time-window of opportunity for young circulating parasites to escape into the central intra-vascular compartment. The escaped parasites are more likely to avoid the parasiticidal action(s) of the drug. It becomes apparent therefore, that co-formulation should hypothetically overcome the inherent disadvantages of the individual drugs.
1) The recommended dosage for adult population (age >16 years) for uncomplicated malaria is a single dose of eight tablets (total dose 1,000 mg artemisinin/400 mg naphthoquine). For children, it is recommended that it be adjusted on a body-weight basis (25 mg artemisinin/10 mg naphthoquine). For younger children, including infants, tablets should be crushed before administration. The manufacturer's current recommendation is that all medications are to be taken before meals or after meals (˜2 h post-prandial) but not with a meal. The following studies were completed: ARCO® vs. chloroquine and sulphadoxine-pyrimethamine (SP) combination. This trial was conducted in an adult population with uncomplicated falciparum malaria infections in Papua New Guinea (Melanesian-Western Pacific). In this setting the ARCO® tablets were administered as a single dose versus chloroquine once a day for three days with a single dose of SP at the start of therapy. The therapeutic responses were monitored for 28 days. Although the two treatments provided relatively comparable cure rates, ARCO® treatment was superior in rate of clearing parasitaemia.
2) ARCO® vs. dihydroartemisinin-piperaquine (DHA-PPQ). This study was conducted in Indonesia in an adult population with uncomplicated falciparum malaria, vivax malaria, and in coinfection of falciparum-vivax malaria. In this study, a single dose (8 tablets) of ARCO® versus 3-days (once/day for 3 days) of DHA-PPQ combination tablets was investigated. The therapeutic responses were monitored for 28 and 42 days. Both treatments provided comparable PCR corrected cure rates for Plasmodium falciparum (ARCO® 99% vs. DHA-PPQ 97%), Plasmodium vivax (ARCO® 99% vs. DHA-PPQ 97%), and mixed infection of Plasmodium falciparum and Plasmodium vivax (ARCO® 79% vs. DHA-PPQ 97%) malarias at day 42. There was no statistically significant difference in parasite clearance times (ARCO® 28±11.7 vs. 26±12.2 DHA-PPQ) for both treatments; however, response to ARCO® treatment was low in mixed infection [source: KPC archived data].
3) ARCO® vs. artemether-lumefantrine (AL). Two of these studies were conducted in children populations of Nigeria and Uganda, and one in an adult population with uncomplicated falciparum malaria in Uganda. For the children, the number of ARCO® tablets administered was based on the bodyweight (25/10 mg/kg artemisinin-naphthoquine combination) and for the adult population ARCO® tablets were administered as a single dose of eight tablets. The therapeutic responses were monitored for 28 days in the Nigerian study and 42 days in the Ugandan study. There was no significant difference in the efficacy and safety profiles in children in the two studies between the single dose ARCO® treatment and 6-dose regimen of AL at day 28 and day 42, respectively. Similar observations were made between the two treatments in the adult population in the Ugandan study at day 28 [Source: KPC archived data].
4) ARCO® vs artemether-lumefantrine vs artesunate-amodiaquine (three-arm study). This study was conducted in Nigeria in mixed population (children+adults) with uncomplicated falciparum malaria. ARCO® tablets were administered according to the dosing schedule described above. For AL, tablets were administered twice a day for three days based on bodyweight for children and predetermined number of tablets for adults while for AA, once a day treatment for 3 days based on bodyweight for children and predetermined number of combination tablets for adults. The therapeutic responses were monitored for 28 days. The study concluded that ARCO® and AA treatments were marginally better than artemether-lumefantrine in these settings [Source: KPC archived data].
5) ARCO® (2x/day in divided dosage) vs. artemether-lumefantrine (AL). This study was undertaken in Ivory Cost, West Africa, in a mixed population (children and adults) with uncomplicated falciparum malaria. Drugs were administered as described above. The therapeutic responses were monitored for 28 days. There was no significant difference in the efficacy and safety of 1-day (2×/day in divided dosage) treatment with ARCO® and 3-day with AL (cure rate: ARCO® 100% vs. 98% AL).
6) ARCO® (single dose) vs ARCO® (2×/day divided doses). This study was conducted in Benin (Central Africa) in a child population with uncomplicated falciparum malaria. Children received number of tablets based on bodyweight as a single dose versus same dose divided into two doses give 12 h apart. The therapeutic responses were monitored for 28 days. Therapeutic efficacy and safety were similar for both therapeutic dose regimens (i.e. both regimens were equally effective).
7) ARCO® alone. Two studies were conducted in the adult populations with uncomplicated falciparum malaria: one in Nigeria and one in Myanmar, with no comparators. The therapeutic responses were monitored for 28 days. Both studies demonstrated high efficacy and safety profile for ARCO® in the respective country settings. The safety data provided in relation to individual patients were primarily clinical. Where laboratory data was available, the laboratory evaluation schedules were not consistent between the studies, making comparative interpretation of safety data difficult. Because the trial methodology included in the pooled analysis had not been prospectively standardized, there existed substantial inter-trial differences in defining, assessing, reporting, and classifying adverse events. Furthermore, reliably distinguishing drug side effects from clinical symptoms of malaria infection is often difficult and much of the reporting is largely dependent upon a subjective assessment performed at the time of the event. The safety data obtained for this analysis were from individual patient data case record forms, which were archived at the Centre for clinical studies at Kunming Pharmaceutical Corporation. A total of 16 different adverse events, with varying frequencies and intensities, were recorded in the pooled analysis of 952 adult patients who received artemisinin-naphthoquine combination for uncomplicated malaria. The five most common adverse events were, in order of frequency, headache, nausea, vomiting, dizziness, and abdominal pain. However, it was difficult to discern which of the adverse events were malaria related and which were due to drug treatment because almost all of these events were reported during the first 24-hrs following the commencement of treatment. No adult or pediatric patients discontinued the treatment or any part of the treatment prematurely due to adverse experiences.

Despite the methodological limitations of this analysis, the overall safety profile of ARCO® treatment in these series of studies appeared to be benign. The total incidence of drug-related adverse events considered by clinicians and/or principal investigators was estimated to be low (≤5%). The majority of the adverse events reported has been of gastrointestinal-related in nature and were self-limiting. In general, safety profile of ARCO® treatment appears to be excellent.

The common adverse events include headache, nausea, vomiting, dizziness, and abdominal pain which are self-limiting. A transient deafness has been reported by some patients. QTc prolongation between baseline and 4 h after the final dose may occur following ARCO® treatment. However, the same has been found to be an adverse effect associated exclusively with naphthoquine. Nevertheless, the drug should not be administered to individuals who are at risk of QTc prolongation, cardiac arrhythmias and in patients with electrolyte imbalance.

While the therapeutic assessment of ARCO® demonstrated high level of efficaciousness and safety, the following gaps in our knowledge exist.
Children: Malaria affects children more than adults. There is, however, insufficient information available on the pharmacokinetics of ARCO® in children, particularly in children between 6 months to 5 years of age.
Pregnancy: Women who are pregnant are relatively more vulnerable to malaria than non-pregnant women. Denying this population the most effective antimalarial drugs available would not save lives. It may be that the embryonic toxicity of ACTs in human pregnancies has been over-emphasized.

### Artemisinin-Piperaquine

As per the PRISMA guidelines, the EMBASE, MEDLINE, the Google Scholar Library, and Cochrane library databases were systematically searched from inception until July 2020 with the following terms: “artemisinin-piperaquine” or “AP.” Only randomized controlled trials (RCTs) Ts) were included. The competing interventions included dihydroartemisinin—piperaquine (DHA-PPQ), artemether-lumefantrine (AL, Coartem), artesunate-melfloquine (ASAM) and artesunate-amodiaquine (ASAQ, Artekin). Singlearm clinical trial on AP was also assessed. The reported outcomes, including the overall response, cure rate, fever and parasite clearance time, hematology, biochemistry, electrocardiogram (ECG), adverse events, recurrence rate, and sensitivity analyses, were systematically investigated. All data were analyzed using the Review Manager 5.3.

A total of seven studies were reviewed, including five RCTs and two single-arm studies. A pooled analysis of 5 RCTs (n=772) revealed a comparable efficacy on polymerase chain reaction (PCR)-confirmed cure rate between AP and competing interventions in treating uncomplicated malaria. As for the fever and parasite clearance time, due to the lack of complete data in some studies, only 3 studies' data could be used. The patients showed good tolerance to all drugs, and some side-effects (such as headache, anoxia, vomiting, nausea, and dizziness) were reported for every group, but they were self-limited and showed no significant difference.

The most common adverse events reported before and after treatment are shown below and they were generally mild in intensity. The percentage of patients reporting adverse events was comparable between the two treatment groups before and after starting treatment. There was a marked decline in adverse events 24 h after the first dose of each ACT and by 48 h after commencing treatment most patients were free of adverse events. AEs are primarily disease related.

A clearly identified safety concern with PQP and other members of the 4-aminoquinoline drug class is the potential to cause QTc prolongation at therapeutic doses and for example, QT prolongation is described in the European Summary of Product Characteristics for Eurartesim®, a DHA-PQP combination. The molecular mechanism for QT prolongation with piperaquine is selective inhibition of the cardiac delayed rectifier current, (also referred to as the hERG channel). Piperaquine in combination with DHA is approved in the EU and in other countries. One tablet contains 320 mg PQP and 40 mg DHA. In Europe, Eurartesim® (DHA-PQP) is indicated for the treatment of uncomplicated falciparum malaria in adults, children and infants 6 months and over and weighing 5 kg or more.

The QTc prolonging property of piperaquine is described and well quantified from studies with DHA-PQP. In a thorough QT study, the QT effect of DHA-PQP was evaluated in healthy subjects and compared with the effect of artemether/lumefantrine. DHAPQP was dosed weight-adjusted (three or four tablets) for 3 days with either a high fat/low calorie meal (group 1, 64 subjects), a high fat/high calorie meal (group 4, 40 subjects) or in the fasted state (group 5, 40 subjects). DHA-PQP caused QTc prolongation. On day 3, the largest by time point observed mean placebo-adjusted ΔQTcF was 45 ms, 36 ms and 21 ms in groups 4, 5 and 1, respectively.

In two recent and yet unpublished observational/phase IV patient studies (INESS and WANECAM), ECGs were recorded at baseline and at the projected piperaquine tmax and the effect was larger: ΔQTcF of approximately 30 ms (data on file, MMV). A substantially larger effect than in the pivotal trials was also observed in a recently published, prematurely halted clinical trial in Cambodia in which a mean placebo-adjusted ΔQTcF of 46 ms was seen at peak plasma concentrations after 2 days of dosing with DHAPQP 180/1440 mg.

These findings were confirmed and further quantified in this study in healthy subjects, in which a statistically significant relationship between piperaquine plasma concentration and placebo-adjusted ΔQTcF was demonstrated with a slope of 0.047 ms per ng ml-1 (90% CI 0.038, 0.057), i.e. approximately 5 ms for every 100 ng ml-1 of increase in piperaquine plasma concentration.

### Artemisinin-Piperaquine Against COVID-19

Forty-one patients with confirmed COVID-19 were enrolled in the study and divided into two groups: artemisinin-piperaquine (AP) group (n=23) and control group (n=18). The primary outcome was the time taken to reach undetectable levels of severe acute respiratory syndrome-coronavirus-2 (SARS-CoV-2) and the percentage of participants with undetectable SARS-CoV-2 on day 7, 10, 14, and 28. The computed tomography (CT) imaging changes within ten days, the corrected QT interval changes, adverse events, and abnormal laboratory parameters were the secondary outcomes.

The mean time to reach undetectable viral RNA (mean±standard deviation) was 10.6±1.1 days (95% confidence interval [CI]: 8.4-12.8) for AP group and 19.3±2.1 days (95% CI: 15.1-23.5) for the control group. The percentage of patients with undetectable viral RNA on day 7, 10, 14, 21, and 28 were 26.1%, 43.5%, 78.3%, 100%, and 100%, respectively, in the AP group and 5.6%, 16.7%, 44.4%, 55.6% and 72.2%, respectively, in the control group. The CT imaging within ten days post-treatment showed no significant differences between the two groups (p>0.05). Both groups had mild adverse events.

AP group: AP (ARTEPHARM Co., Ltd) was used as an antiviral and symptomatic treatment. AP was orally administrated with a loading dose of two tablets (artemisinin 125 mg and piperaquine 750 mg) for the first day and followed by a maintenance dose of one tablet/day (artemisinin 62.5 mg and piperaquine 375 mg) for the next six days. The total dose was eight tablets in 7 days.

Control group: Hydroxychloroquine/Arbidol, according to the “China's Novel Coronavirus Pneumonia Diagnosis and Treatment Plan (Trial Seventh Edition)”, was mainly used as an antiviral and symptomatic treatment. Hydroxychloroquine sulfate (Shanghai Zhongxi Pharmaceutical Co., Ltd.) was orally administered as a loading dose of 800 mg/day for the first three days, followed by a maintenance dose of 400 mg daily for the next five days. Arbidol hydrochloride (CSPC Ouyi Pharmaceutical Co., Ltd.) was orally administrated 600 mg/day for eight days, divided into three doses daily.

When drug doses completed, positive patients would be continued to receive symptomatic treatment, and meet the discharge conditions until two consecutive tests for nucleic acid turn negative. All the patient should be quarantined for 14-day observation after discharge. The quarantine restriction could be lifted if the tests remain negative.

The average age of the AP group and control group patients was 42.7 years and 45.8 years, respectively. 82.6% of the patients in the AP group and 88.9% of the patients in the control group were diagnosed with moderate COVID-19, and the rest were mild COVID-19 patients.

The average time to achieve undetectable SARS-CoV-2 RNA in the AP group was significantly less than that in the control group (AP: 10.6±1.1 days (95% CI: 8.4-12.8), control: 19.3±2.1 days (95% CI: 15.1-23.5)) (p=0.001<0.005). The percentage of the patients to achieve undetectable SARS-CoV-2 at the day 7, 10, 14, 21, and 28 during drug administration in the AP group were 26.1%, 43.5%, 78.3%, 100%, and 100%, respectively, while that in the control group were 5.6%, 16.7%, 44.4%, 55.6% and 72.2%, respectively. Analysis of these data indicated that the elimination rate of SARS-CoV-2 RNA in the AP group was significantly higher than that in the control group (RD=0.28; 95%CI 0.07-0.49). The length of the patient's hospital stay for AP group was 13.3±4.8 days, and 21.3±9.1 days for control group. No patients had been transferred to severe cases.

In the 17 patients of the AP group, the ECG results indicated that the average QTc interval value was 411.94 ms before treatment and 433.59 ms 3-8 days post-treatment. The average prolongation was 21.65 ms (95%CI: 3.58-39.71 ms) after treatment. Twelve patients (70.59%) showed varying degrees of prolongation, 6 (35.29%) showed mild prolongation (<30 ms), 4(23.53%) demonstrated moderate prolongation (30-60 ms), and 2 (11.76%) demonstrated severe prolongation (>60 ms). Furthermore, the paired sample t-test showed significant differences between the two groups (p<0.05). AP treatment did not cause TdP and other arrhythmias in the patients. The patients with prolonged QT interval returned to normal after the treatment was discontinued. ECG changes for the control group patients were not collected and recorded.

Conclusions:
1. Piperaquine and hydroxychloroquine are members of the quinoline family and they both have marginal activity against SARS-CoV-2 in vitro only. HCQ failed clinical trials against COVID-19. Therefore, Artemisinin is the main difference between the two arms.
2. Time to Undetectable SARS-CoV-2 RNA in the AP group was significantly less than the control group (AP: 10.6±1.1 days), Control: 19.3±2.1 days). P value is highly significant (p=0.001).
3. Length of hospital stay for AP group was 13.3±4.8 days, and Control was 21.3±9.1 days.
4. However, the cardiac toxicity of piperaquine would argue against the combine use of this fix dose combination and favoring the use of free Artemisinin.

Artemisinin and/or OT-101 treatment to shut down the TGF-β driven pathologies outlined below:
a) TGF-β recruits neutrophils into the site of inflammation increasing the risk for pulmonary thrombosis.
b) TGF-β inhibits ENaC and causes fluid accumulation in the lung and ARDS/pneumonia.
c) TGF-β induces late stage fibrosis compromising lung capacity even after recovery.
d) TGF-β induces IL-6 leading to systemic inflammation and “cytokine storm.”
e) TGF-β induces TGFBIp leading to vascular inflammation.
f) TGF-β induces IgA class switching leading to IgA vasculitis/Kawasaki Disease syndrome.
g) GF-β induces Furin which increase cellular uptake of the virus. This together with viral induction of TGF-β freezing cellular cycle allowing the virus to replicate forms a positive loop that lead to TGF-β surge which drive the pathologies described for a-g.

### Antisense Oligonucleotide

An antisense oligonucleotide (ASO) is a single-stranded deoxyribonucleotide, which is complementary to the mRNA target. The goal of the antisense approach is the downregulation of a molecular target, usually achieved by induction of RNase H endonuclease activity that cleaves the RNA-DNA heteroduplex with a significant reduction of the target gene translation. Other ASO-driven mechanisms include inhibition of 5′ cap formation, alteration of splicing process (splice-switching), and steric hindrance of ribosomal activity.

Antisense strategies utilize single-stranded DNA oligonucleotides that inhibit protein production by mediating the catalytic degradation of target mRNA, or by binding to sites on mRNA essential for translation. Antisense oligonucleotides can be designed to target the viral RNA genome or viral transcripts. Therefore, ASOs have been widely use in the treatment of viral disease. As, Antisense oligonucleotides (ASOs) provide an approach for identifying potential targets, and therefore represent potential therapeutics.

Coronaviruses make up a large family of viruses that can infect birds and mammals, including human, and have been responsible for several outbreaks around the world, including the severe acute respiratory syndrome (SARS-CoV), the Middle East respiratory syndrome (MERS-CoV), and the most recent novel coronavirus (COVID-19) in Wuhan, China.

SARS-CoV is a virus from genus Coronaviridae, the family of Coronaviridae, which are enveloped, positive-stranded viruses with ˜30,000 nucleotides. These largest RNA viruses are composed of three groups: Group 1 contains transmissible gastroenteritis coronavirus (TGEV), porcine gastroenteritis virus etc.; Group 2 consists of SARS-CoV, mouse hepatitis virus (MHV) etc. and Group 3 contains avian infectious bronchitis virus (AIBV) etc.

The coronavirus is a monopartite, linear single-strand RNA(+) and its genome size ranges from 27 to 32 kb (the largest of all RNA virus genomes). The coronavirus genome is usually capped, and polyadenylated. The leader RNA (65-89 bp) at the 5′ end of the genome is also present at the end of each subgenomic RNAs. The virion RNA is infectious and serves as both the genome and viral messenger RNA. Genomic RNA encodes ORF1a, as for ORF1b, it is translated by ribosomal frameshifting. Resulting polyproteins pp1a and pp1ab are processed into the viral polymerase (RdRp) and other non-structural proteins involved in RNA synthesis. Structural proteins are expressed as subgenomic RNA.

Two-thirds of the SARS-CoV genome encode viral replicase gene which is translated into two overlapping replicase polyproteins pp1a (˜490 kDa) and pp1ab (˜790 kDa). The polyproteins are later cleaved by two viral proteinases, 3Clike protease (3CLpro) and papain-like protease (PLpro), to yield non-structural proteins essential for viral replication. The remaining one-third encode 3CLpro and PLpro are still considered as a viable target, along with some new alternatives, such as E protein (Orf4), M protein (Orf6), N protein (Orf9), Orf3a, RNA-dependent RNA polymerase (RdRp) and 5¢-3¢ helicase.

The life cycle of the virus is as follow:
1) Attachment of the viral S protein (maybe also HE if present) to host receptors mediates endocytosis of the virus into the host cell.
2) Fusion of virus membrane with the endosomal membrane (probably mediated by S2), ssRNA(+) genome is released into the cytoplasm.
3) Synthesis and proteolytic cleavage of the replicase polyprotein.
4) Replication occurs in viral factories. A dsRNA genome is synthesized from the genomic ssRNA(+).
5) The dsRNA genome is transcribed/replicated thereby providing viral mRNAs/new ssRNA(+) genomes.
6) Synthesis of structural proteins encoded by subgenomic mRNAs.
7) Assembly and budding at membranes of the endoplasmic reticulum (ER), the intermediate compartments, and/or the Golgi complex.
8) Release of new virions by exocytosis.

Antisense oligonucleotides (ASO) are small synthetic pieces of single-stranded DNA that are normally 15-30 nucleotides in length. ASOs specifically bind to complementary DNA/RNA sequences by Watson-Crick hybridization and once bound to the target RNA, inhibit the translational processes either by inducing cleavage mechanisms or by inhibiting mRNA maturation. The use of ASOs was first reported by Zamecnik and Stephenson in 1978 as potential antiviral therapeutics. They utilized a phosphodiester oligodeoxynucleotide composed of 13 nucleotides (a 13-mer) that was designed to block Rous sarcoma virus replication. Since then, ASOs ability to selectively inhibit gene expression has generated noteworthy enthusiasm in the scientific and medical community because of its specificity and the breadth of its potential applications as therapeutic agents. An extensive range of oligonucleotide analogs has become available over the past decade and this led to target validation and development of ASO-based antiviral agents whose efficacy have been reported against various virus types, both in vitro as well as in vivo. For ASOs to be used empirically, modifications of DNAs or RNAs were needed to retain hybridization capacity at the same time increasing stability.

Major alterations have been introduced in the phosphodiester bond, the sugar ring, and the backbone to result in three generations of nucleic acid analogs for the synthesis of ASO oligomers. ASO-based antiviral agents are specifically designed to block the translational processes either by (i) ribonuclease H (RNAse H) or RNase P mediated cleavage of mRNA or (ii) by sterically (non-bonding) blocking enzymes that are involved in the target gene translation.

Antisense Oligonucleotide against Negative-Sense and Single-Stranded RNA Virus Influenza

Antisense oligonucleotides have been studied extensively against several respiratory viruses with promising results. The earliest studies using oligonucleotide (oligo') to inhibit synthesis of virus-specific proteins, including influenza, in MDCK cells were reported in the 1990s. Researchers observed that the modified oligos could effectively suppress the influenza A/PR8/34 (H1N1) virus production. Since then several other ASOs have been synthesized and studied for efficacy against influenza. Ge et al. showed that siRNAs specifically designed to target the conserved regions of the viral genome can potently inhibit influenza virus production in cell lines (Vero, MDCK) as well as embryonated chicken eggs.

Wu et al. showed that in vivo treatment with three doses of RNA oligonucleotides conferred significant protection to the infected chickens from H5N1 virus-induced mortality at a rate of up to 87.5%. The authors used mixed RNA oligonucleotides targeting the NS1 gene to show a significant reduction in the plaque-forming unit (PFU) and viral RNA levels in the lung tissues of the infected animals by plaque assay and real-time PCR analysis. Their study demonstrated that RNA oligonucleotides targeting at the viral NS1 gene could potently inhibit highly pathogenic avian H5N1 influenza virus reproduction and thus, could potentially be used as prophylaxis and therapy for H5N1 influenza virus infection in humans.

In another study, Gabriel et al. used three peptide-conjugated phosphorodiamidate morpholino oligomers (PPMOs), to selectively target the translation start site region of PB1 or NP mRNA or the 30-terminal region of NP viral RNA, to prevent virus replication in MDCK cells. The study further utilized the primer extension assays to show that treatment with any of the effective PPMO markedly reduced the levels of mRNA, cRNA, and vRNA.

Another study by Duan et al. used a novel antisense oligonucleotide (IV-AS) specifically designed against the 50-terminal conserved sequence found in all the eight viral RNA segments of influenza A virus. They monitored the activity of IV-AS both in vitro in the MDCK cells and in vivo using a mouse model. IV-AS was administered intranasally to the H5N1-infected mice once daily for 6 days starting 6 h post-infection. IV-AS, at 50% effective concentration (EC50) ranging from 2.2 to 4.4 uM, inhibited influenza A virus-induced cytopathic effects in MDCK cells. IV-AS was also effective against the H5N1 virus in preventing death, reducing weight loss, reducing lung consolidation and decreasing the lung virus titers.

It is known in the literature that antisense-PPMOs, delivered through the intranasal route, were able to inhibit the replication of equine influenza A virus A/Eq/Miami/1/63 (H3N8) in mice by over 95% compared to the controls. In another study, a group of authors designed antisense oligonucleotides against the common 30 NCR of segments of the IAV genome to inhibit its replication. The AS molecules demonstrated a drastic reduction in the cytopathic effect caused by A/PR/8/34 (H1N1), A/Udorn/307/72 (H3N2), and A/New Caledonia/20/99 (H1N1) strains of IAV for almost 48 h post-infection. The same AS molecule protected mice against all the strains of the influenza virus.

Phosphorothioate oligonucleotides (S-ONs) obtained from the packaging signals in the 30 and 50 ends of the PB2 vRNA have been tested against the influenza virus in vitro. The 15-mer S-ON (designated 5-15b) derived from the 50 end of the PB2 vRNA, and complementary to the 30 end of its coding region (nucleotides 2279-2293), proved noticeably inhibitory. Similar to other related studies, the antiviral activity of 5-15b was also observed to be dose- and time-dependent; however, it was independent of the cell substrate and multiplicity of infection used in the study.

In another follow-up study, it has been investigated whether analogous inhibitory S-ONs targeting PB1 and PA gene segments could be identified and if the virus can develop resistance to S-ONs. The authors observed that the 20-mer S-ONs reproducing the 50 ends of PB 1 and PA gene segments exerted a dominant antiviral activity against several influenza A virus subtypes in MDCK cells. Their findings suggest that the packaging signal at the 50 end of the PB2 vRNA can be a potential therapeutic target for the design of novel anti-influenza compounds. Also, antivirals against this region could be beneficial owing to fewer changes of mutation in these viral genes.

Lenartowicz et al. designed and tested 20-0-methyl and locked nucleic acid antisense oligonucleotides (ASOs) to specifically target the internal regions of influenza A/California/04/2009 (H1N1) viral RNA segment 8. Of the 14 designed and tested ASOs, 10 showed significant inhibition of viral replication in MDCK cells. The ASOs were 11-15 nucleotides long and demonstrated varying inhibition ranging from 5- to 25-fold. The designed ASOs were very specific for IAV and showed no inhibition of influenza B/Brisbane/60/2008 (Victoria lineage). The combinations of ASOs slightly improved anti-influenza activity. These studies show that ASOs can be designed to be accessible to IAV RNA in regions other than the panhandle formed between the 50 and 30 ends.

### Respiratory Syncytial Virus (RSV)

Linear negative-sense RNA genome including RSV has also been targeted by several designed ASO molecules. Jairath et al., in their study investigated the use of oligodeoxyribonucleotides to inhibit RSV replication in cell culture. Human epithelial type 2 (HEp-2) cells were infected with RSV strain A2 and treated with the designed oligonucleotides. A 0.5-1 uM50% effective concentration (EC50) values were obtained for the designed antisense oligonucleotide targeted to the start of the viral NS2 gene. The ELISA and PT-PCR analyses showed that all the oligonucleotides inhibited virus antigen production and demonstrated sequence-specific depletion of the genomic RNA target. The target RNA was observed to be cleaved at the specific antisense oligonucleotide binding site. The results suggest that antisense oligonucleotides could have therapeutic value against RSV infections. PPMOs have the ability to readily enter cells and interfere with viral protein expression through steric hindrance of the complementary RNA. Lai et al. designed two antisense PPMOs to specifically target the 50-terminal region and translation start-site region of RSV L mRNA. Both PPMOs demonstrated minimal cytotoxicity when tested for anti-RSV activity in two human-airway cell lines. One PPMO (AUG-2), reduced the viral titers by >2.0 log10. Intranasal administration of AUG-2 in BALB/c mice before the RSV infection showed a reduction in viral titer of 1.2 log 10 in lung tissue at day 5 post-infection, and further reduced pulmonary inflammation at day 7 post-infection. The overall results show that PPMO has a potent anti-RSV activity and the potential to be a therapeutic regimen against RSV infections.

### Ebola

Ebola virus is a highly pathogenic filovirus causing severe hemorrhagic fever with high mortality rates. It assembles heterogenous, filamentous, enveloped virus particles containing a negative-sense, single-stranded RNA genome packaged within a helical nucleocapsid (NC). The viral genome encodes for a nucleoprotein (NP), glycoprotein (GP), RNA dependent RNA polymerase (L), and four structural proteins termed VP24, VP30, VP35 and VP40. In addition, the Ebolavirus is able to express a truncated soluble form of GP (sGP) through RNA editing. Currently, the most promising studies in the antiviral treatment of EBOV use the ability of virus-gene-specific oligonucleotides to interfere with the translation of viral mRNA. This antisense strategy inhibits EBOV replication, resulting in a reduction in the pathogenic effects of EBOV and allowing the immune system more time to clear the infection. A combination antisense strategy targeted at ZEBOV L, VP24 and VP35 was an efficacious post-exposure treatment in several rodent and NHP studies (Warfield 2006, Warren 2010). Depending on the time of intravenous treatment, 66% or 100% of NHPs were protected from a lethal challenge (Gelsbert 2010). A similar study, using chemically modified oligonucleotides called phosphorodiamidate morpholino oligomers, conferred 60% protection against lethal ZEBOV challenge in NHPs following intraperitoneal, subcutaneous and intravenous administration (de Wit 2011).

### Antisense Oligonucleotide Against Positive-Sense and Single-Stranded RNA Virus Severe Acute Respiratory Syndrome (SARS)

Neuman et al. designed specific PPMOs against specific sequences in the SARS-CoV (Tor2 strain) genome. The PPMOs were analyzed for their capability to inhibit infectious virus production and were further investigated to determine the function of conserved vRNA motifs and their secondary structures. Several virus-specific PPMOs along with a random-sequence control PPMO were designed that showed low inhibitory activity against SARS-CoA. The virus-targeted PPMOs further reduced the cytopathology due to viral infection and reduced cell-to-cell spread because of a reduction in viral replication. The active PPMO was found to be most effective when administered any time prior to the peak viral synthesis and exerted a sustained antiviral effect in the culture medium. The study demonstrated the antiviral effects in vitro for the PPMO designed complementary to the AUG translation start site region of a murine coronavirus replicase and suggested a therapeutic potential of ASOs against coronavirus infection. In another study, Ahn et al. evaluated the antiviral effects of antisense peptide nucleic acids (PNAs) targeting a highly conserved RNA sequence on the programmed 1 ribosomal frameshifting (1 PRF) that is utilized by eukaryotic RNA viruses. Cells transfected with a SARS-CoV-replicon, treated with the PNA (50% inhibitory concentration of 4.4 uM) fused to a cell-penetrating peptide (CPP), showed a significant suppression of the replication of the SARS-CoV.

Fukuda et al. in their patent and paper describe ribozyme, an antisense RNA molecule with catalytic activity, for the treatment of infections by SARS-CoV and other CoVs like MHV. This ribozyme specifically recognizes the base sequence, namely GUC, present in the loop region, on the mRNA of SARS-CoV or other HCoVs. The complementary base sequence on the ribozyme is derived by deleting, adding or modifying bases without altering its binding affinity. A Chinese patent has claimed to use small interference RNA to inhibit SARS-CoV's M protein expression. The designed double-stranded RNA, named siRNA-M1, has sequence of 5′-gggugacuggcgggauugcgau-3′, complementary to the sequence of M protein mRNA 220-241 nucleotides. Another siRNA-M2, 5′-gggcgcugugacauuaaggac-3′, is complementary to the 460-480 nucleotides of M protein mRNA. These two siRNAs were shown to inhibit the expression level of M protein mRNA.

In another study by Shi et al, Vero E6 cells were transfected with plasmid constructs containing exons of the SARS-CoV structural protein E, M or N genes or their exons in frame with the reporter protein EGFP. The transfected cell cultures were treated with antisense phosphorothioated oligonucleotides (antisense PSODN, 20 mer) or a control oligonucleotide by addition to the culture medium. Among a total of 26 antisense PS-ODNs targeting E, M and N genes, six antisense PS-ODNs were obtained which could sequence-specifically reduce target genes expression by over 90% at the concentration of 50 μM in the cell culture medium tested by RT-PCR. The antisense effect was further proved by down-regulating the expression of the fusion proteins containing the structural proteins E, M or N in frame with the reporter protein EGFP.

In Vero E6 cells, the antisense effect was dependent on the concentrations of the antisense PS-ODNs in a range of 0-10 μM or 0-30 μM. The method of administration of the antisense oligo is crucial for the inhibition effect obtained in Vero E6 cells. The down-regulation effect of antisense PS-ODN added to the culture medium as a free oligonucleotide is varied between different cell types. This could be due to different intracellular concentrations of the PS-ODN, cell-type-specific differences in the level of RNase H, which is supposed to be the main factor in antisense inhibition of gene expression mediated by PS-ODNs.

### Mouse Hepatitis Virus (MHV)

Burrer et al. studied the effect of PMO compound on MHV replication and disease in vivo. Ten P-PMOs directed against various target sites in the viral genome were tested in cell culture, and one of these (5TERM), which was complementary to the 5 terminus of the genomic RNA, was effective against six strains of MHV. Further studies were carried out with various arginine-rich peptides conjugated to the 5TERM PMO sequence in order to evaluate efficacy and toxicity and thereby select candidates for in vivo testing. In uninfected mice, prolonged P-PMO treatment did not result in weight loss or detectable histopathologic changes. 5TERM P-PMO treatment reduced viral titers in target organs and protected mice against virus-induced tissue damage. Prophylactic 5TERM P-PMO treatment decreased the amount of weight loss associated with infection under most experimental conditions. Treatment also prolonged survival in two lethal challenge models. In some cases of high-dose viral inoculation followed by delayed treatment, 5TERM P-PMO treatment was not protective and increased morbidity in the treated group, suggesting that P-PMO may cause toxic effects in diseased mice that were not apparent in the uninfected animals. However, the strong antiviral effect observed suggests that with further development, P-PMO may provide an effective therapeutic approach against a broad range of coronavirus infections.

Important Findings for the Development of Antisense against SARS/Coronavirus Viral Mutation in Response to Antiviral Therapy

The error-prone replication of RNA viruses plays an important role in viral evolution and drug resistance. One of the challenges for drug development is the propensity for virus to mutate in response to antiviral agents and result in drug resistance. Many studies have been done to show the propensity for SARS-CoV to develop resistance to antiviral agents, including antisense. In the study done by Neuman et al., the researchers reasoned that antiviral effects of P-PMO might be improved by choosing conserved RNA sequence elements and secondary structures critical for replication, transcription, and host factor interaction as targets. They demonstrated that antisense-mediated suppression of viral replication can be achieved by targeting conserved RNA elements required for viral RNA synthesis and translation. P-PMOs tested included five designed to directly inhibit translation of the replicase open reading frame la (TRS1-2, AUG1-3), one to inhibit ribosomal frameshifting (1ABFS), three to bind conserved sequences in the 3′-untranslated region (3UTR, S2M, 3TERM), and one scrambled control sequence (DSCR). P-PMOs directed to the leader transcription regulatory sequence were most effective at reducing viral titer.

SARS-CoV plaque purified after 11 rounds of TRS2 P-PMO selection formed small plaques on Vero-E6 cells in the absence of P-PMO. TRS2 P-PMO-selected SARS-CoV displayed delayed growth kinetics compared with untreated SARS-CoV and other P-PMO-selected SARS-CoV. RNA was isolated from plaque purified SARS-CoV selected after 11 rounds of serial P-PMO treatment. RT-PCR amplicons from 14 serially P-PMO-treated SARS-CoV were sequenced to determine whether the virus had undergone mutation during P-PMO selection. Three contiguous base changes of CTC to AAA at position 61-63, proximal to the leader TRS and within the target region of TRS2-P-PMO, appeared in only the 14 amplicons from TRS2-resistant SARS-CoV.

Thermal melting curve data for peptide-conjugated PMO/RNA duplexes with variable mismatches lead them to speculate that the three mutations at the TRS2-P-PMO target site reduce the effective melting temperature (Tm) by ˜25-30° C.

### Target Sequence Should be the Conserved Sequence Among All the Variants of the Virus

Zhang et al. reported that siRNAs targeting the S gene could inhibit SARS-CoV replication and questioned whether the siRNA targeting the Leader sequence was more effective than targeting a specific gene. Leader sequence was highly conserved between different strains of SARS-CoV, while the other sequences coding for the specific proteins such as S, N, M and E contained various mutations between the different strains. According to this information, the sequence of CCAACCAACCTCGATCTC was selected and designed as the siRNA target for the proper GC/AT ratio and CC structure.

To compare the effectiveness, they used the same amounts of plasmids to transfect Vero E6 cells and measured the virus titers in the supernatant of the cells infected by the same amounts of SARS-CoV. The data show that the virus titer was 4.4×106 PFU in the infected cells with U6/GFP-RNAi plasmid, while the virus titers decreased to 4.2×105, 4.8×105 and 7.8×104 PFU in the cells with U6/S-RNAi1, U6/SRNAi2 and U6/L-RNAi plasmids, respectively.

The probability of gene variation in the S gene might cause randomly selected targeting sequence changes, which would reduce the effectiveness of the designed siRNA. While the Leader sequence of the coronavirus remained identical, they postulated generating siRNA targeting the Leader sequence of SARS-CoV, which is necessary for the transcriptions of various genes of the virus. This targeting site was more powerful than targeting individual genes and would overcome the various mutations of the other genes in SARS-CoV.

### Targeting the Genomic RNA Exclusively is More Efficient (TRS1 and 5TERM)

To prevent viral mutation resulted from antisense therapy, the conserved regions between different strains of SARS-CoV should be used as the target sequences for antisense. The 5′UTR sequences of different SARS-CoV isolates are relatively conserved, and a full sequence would form a secondary structure containing four stem-loop domains. The cDNA sequence corresponding to SARS-CoV 5′UTR possessed a promoter activity in eukaryotic cells. The promoter domain of the SARS-CoV 5′UTR contains both stem-loop I and II. The 56th nucleotide and its downstream TRS of SARS-CoV 5′UTR plays a key role in regulating transcription. Cells sourced from various tissues can provide efficient accessory factors for the SARS-CoV 5′UTR sequence that acts as a promoter, and the lung-sourced cells may be the most suitable.

Neuman et al. studied the effects of antisenses targeting on different regions of SARS-CoV RNA. In the study, P-PMO were designed to target conserved viral sequences implicated in SARS-CoV RNA synthesis, translation, and/or host factor interaction. The expression of the coronavirus replicase polyprotein is controlled at two points: the initiation of translation at open reading frame 1a, and the ribosomal frameshift which results in translation of the extended open reading frame 1ab. Three sequences were selected in the immediate vicinity of the AUG translation-initiation codon of the viral replicase polyprotein open reading frame 1a (AUG1, AUG2, and AUG3) such that AUG2 and AUG3 overlapped the initiation codon and AUG1 was located in the 5untranslated region proximal to the translation start site. P-PMO 1ABFS was designed to disrupt the RNA secondary structure at the 1 ribosomal frameshift site that mediates translation of the remainder of the replicase polyprotein. The untranslated 5-terminal 263 nucleotides of the SARS-CoV RNA also contain the 80-nucleotide leader sequence found at one terminus of each of the 5- and 3-coterminal subgenomic viral RNA species produced in the infected cell. The transcription regulatory sequence (TRS) located in the 5-UTR of the genome is believed to participate in discontinuous RNA synthesis. The leader TRS was targeted with two P-PMO, each designed to mask the consensus TRS (5-CGAAC-3) and disrupt the stem-loop predicted to form in this region. TRS1 is complementary to the TRS in the leader RNA present on both genomic and subgenomic RNA species. TRS2 spans the junction between the leader and a portion of the 5-UTR not present on subgenomic RNAs.

Studies of coronavirus defective-interfering RNAs have shown the genomic termini contain several conserved motifs, some of which act as discrete signals for RNA replication. P-PMO compounds designed against targets in the 3-untranslated region included 3UTR, targeting a portion of the conserved RNA stem-loop/pseudoknot found in most coronavirus genomes; S2M, targeting the stem-loop 2 motif region related to sequences in astroviruses and equine rhinovirus; and 3TERM, targeting the 3terminus of the genomic RNA, including the first five bases of the polyadenosine tail. Two nonsense P-PMO, DSCR and FT, were included to control for nonspecific P-PMO effects. The 5termini of P-PMO were conjugated to an arginine-rich delivery peptide [R9F2; ] or to a rearranged R5F2R4 peptide, which confers equivalent delivery and efficacy properties. The R9F2 and R5F2R4 peptide conjugates were used interchangeably in the antiviral studies presented here. We did not observe detectable differences in sequence-specific or nonspecific effects between PMO conjugated to one or the other of the two delivery peptides.

The most effective P-PMO targeted the transcription regulatory sequence of which the most effective P-PMO was found to be TRS2. Two different P-PMO, TRS1 and TRS2, showed the highest levels of antiviral activity compared to all other P-PMO used in this study. The 20-mer TRS1 and 21-mer TRS2 vary by only a few nucleotides, but are predicted to vary considerably in the targets to which they can bind. The TRS1 target includes the consensus TRS core sequence ACGAAC and 14 bases in the viral 5direction. TRS2 covers the TRS core, four bases in the 3direction, and 11 bases on the 5side. This difference is predicted to allow binding of TRS1 to full-length genomic RNA and all eight of the subgenomic mRNAs. Out of the eight SARS subgenomic RNAs, five have start codons either adjacent to or within two bases of the TRS core. The 3end of the TRS core is also the 3end of the TRS1 target. TRS1 was therefore expected to have a more profound antiviral effect due to its potential for translational inhibition via duplexing to a region immediately upstream of the AUG translation start sites of at least five discrete viral RNAs combined with its potential ability to block discontinuous transcription of all subgenomic minus-strand RNAs. The TRS2 P-PMO spans the flanking sequence on both sides of the TRS core more extensively than TRS1 P-PMO and may therefore be more effective at inhibiting discontinuous transcription. The observation that TRS2 is more efficacious than TRS1 suggests that targeting the genomic RNA exclusively is a more efficient antiviral strategy with this class of antisense compound.

Mouse hepatitis virus (MHV) is a close phylogenetic relative of SARS-CoV. Similar to SARS-CoV, the 5′-ends of the genomic RNA and all mRNA species in mouse hepatitis virus (MHV) contain a leader sequence of approximately 70 nucleotides. Furthermore, 5TERM was more effective than TSR1 again reinforcing that targeting the genomic RNA exclusively is a more effective approach. The relative effectiveness of R9F2-5TERM, R9F2-TRS 1, and R9F2-RND were tested against a panel of MHV strains. Preinfection treatment of cells with R9F2-5TERM reduced titers of five MHV strains over 10-fold, with the strongest effects observed against MHV-A59 and MHV-3. R9F2-TRS1 treatment was less effective than R9F2-5TERM treatment at reducing viral titers, and R9F2-RND treatment slightly increased the release of infectious virus in several cases (Table 2 below). Similar study done with SARS shown that 5TERM and TSR1 were effective, but TSR1 was more effective suggesting that combination of 5TERM and TSR1 would be preferred.

### Ribosomal Frameshifting (−1 PRF) as Potential Target

The genome of SARS-CoV consists of a single-stranded, plus-sense RNA approximately 30 kb in length. The large SARS-CoV RNA genome produces eight 3′-co-terminal, nested subgenomic mRNAs (sg-mRNAs) for the efficient translation of structural and accessory proteins. The 5′ two-thirds of the SARS-CoV genome encode two large replicase polyproteins, expressed by open reading frames (ORF) 1a and 1b. As in other coronaviruses, ORF1a and ORF1b are slightly overlapped and, because ORF1b lacks its own translation initiation sites, the proteins encoded by ORF1b are only translated as a fusion protein together with ORF1a by programmed −1 ribosomal frameshifting (−1 PRF). The ORF1a and ORF1a/1b fusion proteins are proteolytically cleaved into 16 mature nonstructural proteins (nsps) that play multiple crucial roles during viral genome replication. The −1 PRF is thought to be essential for CoV genome replication because the coronavirus RNA-dependent RNA polymerase (RdRp), the key component of the replicase required for viral genome replication, is the first part of the ORF1a/1b protein synthesized after frameshifting.

Natural ribosomal frameshifting hardly occurs during translation. However, PRF, occurring by specific signals, increases the possibility of tRNA slippage up to 50%. The ribosomal frameshift signal consists of two elements, a heptanucleotide slippery site and a downstream tertiary RNA structure in the form of an RNA pseudoknot. SARS-CoV initiates −1 frameshifting at the three-helix-containing RNA pseudoknot. Recently, control of −1 PRF efficiency has been shown to be critical for the maintenance of correct stoichiometric ratios of viral replicase proteins. The −1 PRF signal is conserved in sequence and structure, which may constrain the ability of SASR-CoV to develop drug-resistant mutants, making it an attractive target for antiviral drug discovery.

Antisense peptide nucleic acids (PNAs) have high hybridization affinity due to their neutral backbones. PNAs also exhibit superior stability compared with other anti-sense agents due to nuclease resistant properties resulting from the replacement of the deoxyribose phosphate backbone with a polypeptide backbone. In the study done by Ahn et al., they designed PNAs that target the pseudoknot structure of the SARS-CoV frameshifting signal, and tested the ability of these molecules to inhibit −1 PRF and SARS-CoV replication using a SARS-CoV replicon expressing a luciferase reporter.

10 μM Tat-FS PNA inhibited viral replication by 82%, whereas Tat-conjugated J3U2 PNA targeting the 3′-UTR of Japanese encephalitis virus (JEV) genome did not affect the viral replication at the same concentration. Consistent with the EMSA and frameshifting reporter assay results, the two-nucleotide mismatched PNA Tat-FSm2 showed a dramatically reduced antiviral activity. These results together clearly demonstrated sequence-specific inhibition of −1 PRF by Tat-FS PNA. In comparison, IFN-β 1a, a potent interferon in reducing SARS-CoV replication in vitro, reduced the luciferase activity by 46% when the replicon-replicating cells were treated with 250 IU/ml IFN-β Synthetic double-stranded RNA Poly(I:C), which triggers type I IFN (α/β) production, also led to suppression of SARS-CoV replication. Tat-FS PNA suppressed SARS-CoV replication in a dose-dependent manner, with an IC50 value of 4.4 μM.

In Vivo Prophylactic Treatment is More Effective than Therapeutic Treatment

Burrer et al. evaluated 10 MHV P-PMOs in cell culture experiments and found that one, with a sequence complementary to the 5-terminal sequence of the viral genome (5TERM), consistently generated the highest level of specific inhibition against each MHV strain challenged. In vivo, 5TERM P-PMO decreased viral replication in the livers of animals infected with various strains of MHV. Histologic examination revealed that the reduction in the severity of liver tissue damage corresponded with a decreased viral load. Prophylactic treatment with 5TERM P-PMO resulted in an improved clinical status of animals after i.p. challenge with each of the three strains of MHV at all inoculum doses tested. However, morbidity and mortality, paradoxically, increased when the administration of P-PMO treatment was delayed until 1 day after infection with high doses of MHV-A1b139. Similar results were obtained with MHV-1 in the lung, where certain antiviral and ineffective P-PMO regimens aggravated clinical disease compared to that in infected controls, irrespective of the level of viral replication. These results collectively reveal both the antiviral activity and potential toxicity of P-PMO treatment in therapeutically relevant MHV challenge models.

### Phosphorothioate (PS) Antisense is Effective

In another study, Hayashi et al. showed that phosphorothioate oligodeoxynucleotides (PS-oligo) complementary to a leader RNA of mouse hepatitis virus (MHV) were more effective inhibitors of MHV multiplication than natural oligodeoxynucleotides (PO-oligo). Sequence-dependent inhibition of viral multiplication was shown at low concentrations (0.001-0.1 μM) of antisense PS-oligo. Phosphorothioate oligodeoxycytidine. PS-(dC)20 and PS-oligo, which has no significant homology to the MHV sequence, showed inhibitory effects on MHV multiplication at concentrations higher than 0.5 μM. These results showed that PS-oligo was more potent than PO-oligo in inhibition of MHV multiplication and that PS-oligo may inhibit MHV multiplication by two different mechanisms, that is, in sequence-dependent and -independent manners.

Hayashi et al. selected the leader sequence including the conserved sequence as a target region for antisense PS-oligo and investigated the effect of PS-oligo on MHV multiplication.

AL-oligo (5′-AAAGTTTAGATTAGATTAGA-3′) (SEQ ID NO:1) contained a sequence complementary to the conserved sequence of a leader RNA of JHMV. ML-oligo (5′-AAAGTTTAGATTAGATTAGA-3′) (SEQ ID NO:2) contained a sequence with 70% homology to AL-oligo.

Random-oligo (5′AAAGTTAATGTAATGTTAGA3′) (SEQ ID NO:3) contained no significant homology with MHV sequences as yet reported. Phosphorothioate oligodeoxycytidine, PS-(dC)20 (5′CCCCCCCCCCCCCCCCCCCC3′) (SEQ ID NO:4) were also synthesized.

The yields of infectious virion particles from the cells treated with AL-oligo and ML-oligo at 0.001 μM were reduced significantly compared with the yields from control cells untreated with PS-oligo. At 0.1 and 0.5 μM, the viral multiplication was inhibited more than 95%. Since no inhibitory effect on the viral multiplication was observed at 1 μM after treatment with natural PO-oligo complementary to the leader RNA, PS-oligo was 1,000 times more potent than unmodified PO-oligo. It has been reported that PS-oligo is more resistant to nuclease digestion in cells and in the whole body. Therefore, PS-oligo might more effectively inhibit viral multiplication in infected cells than PO-oligo did. Although, ML-oligo contained a sequence only 70% homologous to AL-oligo, no significant difference was observed in inhibitory effects on MHV multiplication between AL-oligo and ML-oligo. The reason why there was no significant difference between AL-oligo and ML-oligo in spite of the difference of homology remains unclear. It is well known that the sequence of the 5′ end is important in hybrid formation between the oligonucleotide and the template. Since AL-oligo and ML-oligo have the same sequences at both 5′ and 3′ ends, the efficiency of hybrid formation between AL-oligo and the leader RNA may be similar to that between ML-oligo and the leader RNA. Random-oligo which contained no sequence with significant homology to MHV genes and oligodeoxycytidine, PS-(dC)₂₀, showed inhibitory effects on viral multiplication. The percentages of inhibition by random-oligo and PS-(dC)₂₀ were significantly lower than those by AL-oligo and ML-oligo at low concentration.

One of the anti-sense nucleotide known for TGF-beta inhibition is OT-101 (trabedersen). Trabedersen is a synthetic antisense oligodeoxynucleotide designed to block the production of TGF-beta2, a secreted protein that can exert protumor effects. Trabedersen is indicated for the treatment of malignant brain tumors and other solid tumors overexpressing TGFbeta2, such as those of the skin, pancreas and colon.

Coronavirus entry into cells is follow by suppression of cellular replication and redirection of cellular machineries to the replication of the virus. SARS-CoV-1 infection of VeroE6 cells inhibits cell proliferation by both the phosphatidylinositol 3′-kinase/Akt signaling pathway and by apoptosis. The nucleocapsid protein of SARS-CoV-1 inhibits the cyclin-cyclin-dependent kinase complex and blocks S phase progression in mammalian cells including VeroE6. And, SARS-CoV-1 7a protein blocks cell cycle progression at G0/G1 phase via the cyclin D3/pRB pathway of HEK293, COS-7, and Vero cells. Murine coronavirus replication induces cell cycle arrest in G0/G1 phase in infected 17C1-1 cells through reduction in Cdk activities and pRb phosphorylation.

Infection of asynchronous replicating and synchronized replicating cells with the avian coronavirus infectious bronchitis virus (IBV) arrests infected cells in the G1/M phase of the cell cycle.

RSV infection induces TGF-β expression resulting in cell cycle arrest in A549 and PHBE cells. Cell cycle arrest was also shown to enhance RSV replication. Cell cycle arrest can be reversed by blocking with TGF-β antibody or by TGF-β receptor signaling inhibitor suggesting a role of TGF-β in viral-induced cell cycle arrest. Finally, blocking of TGF-β also resulted in significantly reduced viral protein expression and lower virus titer. In similar fashion, we hypothesized that OT-101′s ability to down-regulate TGF-β2 would affect cell cycle regulation following SARSCoV-1 and SARS-CoV-2 infections, resulting in neutralization of the viruses. Thus, we tested OT-101, an antisense against TGF-β2, in the viral replication assay for both SARS-CoV-1 and SARSCoV-2 (the COVID-19 virus). OT-101 exhibited nM inhibition of both SARS-CoV-1 and SARSCoV-2.

This forms the basis for our IND for OT-101 against COVID-19. Additionally, TGF-β2 is a multifunctional cytokine, playing an important role in the pathology of respiratory viral infection including neutrophil recruitment which could result in the inflammation and pulmonary fluid accumulation that often result in death from COVID-19. Low level of TGF-βs, especially TGF-β2, induces neutrophil chemotaxis to damaged tissue i.e. the lung. As inflammation unfold, high level of TGF-β could contribute to viral pathogenesis through both local phenotypic effects and secondary effects including changes in vascular permeability, resulting from the induction of VEGF or other TGF-β regulated cytokines, chemokines, and growth factors as was shown for Ebola. Together with its demonstrated antiviral activity, OT-101 could be an effective therapeutic against COVID-19.

There is a clear need for new drugs (both preventive and therapeutic) for treatment of patients with COVID-19. This invention is around this novel concept of TGF-β surge as the central cause of COVID-19 pathologies and clinical manifestations. The invention relates to the method of use of known TGF-β inhibitors such as OT-101 and artemisinin for the treatment of COVID-19. It is envisioned that such TGF-β inhibitor would work along the entire three phases of COVID-19 infection: 1) inhibition of viral uptake and/or replication, 2) Inhibition of viral symptoms and 3) inhibition of lung damage on recovery. The present invention overcomes the limitations of prior art and fulfills the need of preventive and therapeutic treatment for viral diseases including COVID-19 by proposing various compositions, methods of treatment and methods of use.

### BRIEF SUMMARY

The present invention provides TGF-beta inhibition by administering agent selected from the group comprising of Artemisinin, OT-101 antisense oligonucleotide.

The present invention provides composition comprising the agents selected from the group comprising of Artemisinin, OT-101 antisense oligonucleotide and other anti-sense oligonucleotides.

The present invention provides TGF-beta inhibition by administering Artemisinin. The present invention provides TGF-beta inhibition by administering OT-101.

The present invention provides a substantially pure Artemisinin having a purity of more than 90%. The present invention provides a substantially pure Artemisinin free of the impurity Thujone.

The present invention provides a substantially pure Artemisinin with negligible amount of the impurities such as Artemisinin, 9-epiartemisinin.

The present invention provides Artemisinin for use in the treatment or prophylaxis of viral or pulmonary diseases.

The present invention provides Artemisinin for use in the treatment of COVID-19. The present invention provides OT-101 for use in the treatment of COVID-19.

The present invention provides composition comprising the agents selected from the group comprising of Artemisinin, OT-101 antisense oligonucleotide along with one or more additional therapeutic agents.

The present invention provides method of treating a fibrosis or any collagen related diseases, cancers, viral diseases, bacterial diseases, fungal diseases, parasite born diseases by administering to a subject agent selected from the group comprising of Artemisinin, OT-101 antisense oligonucleotide and other anti-sense oligonucleotides optionally with one or more additional therapeutic agents.

The present invention provides a method of treating COVID-19 by administering to a subject agent selected from the group comprising of Artemisinin, OT-101 antisense oligonucleotide and other anti-sense oligonucleotides optionally with one or more additional therapeutic agents.

The present invention provides a method of treatment by administering the agents by intravenous, intrathecal, intramuscular, oral, and any other acceptable route of administration.

The present invention provides a pharmaceutically acceptable oral dosage form comprising artemisinin.

The present invention provides a process of extraction of artemisinin.

The present invention provides a composition of matter comprising artemisinin.

The present invention provides a composition of matter of derivatives of artemisinin such as artemether (ARM), artesunate (ARS) and dihydroartemisinin.

The present invention provides Artemisia annua extract comprising Artemisinin, Artemisitene, 9-epiartemisinin and Thujone.

The present invention provides the composition of matter comprising Artemisinin formulated as drug product.

The present invention provides a composition of matter comprising an anti-sense oligonucleotide OT-101 or OT-101 in combination with anti-sense oligonucleotide sequence selected from SEQ ID NOS:5-12 wherein the backbone is modified as OME or LNA, pharmaceutical composition thereof, and use thereof in treatment of viral diseases including COVID-19.

The present invention provides a method of treating TGF-beta storm.

The present invention provides a method of use of anti-sense oligonucleotide by suppression of TGF-beta induced proteins including IL-6, TGFBIp.

The present disclosure broadly lies in the field of pharmaceutics, particularly, TGF-beta inhibition. Specifically, the present invention relates to TGF-beta inhibition utilizing certain agents such as Artemisinin, antisense oligonucleotides. The present invention also provides the composition comprising the said agents, method of treatment and method of use involving said agents.

### BRIEF DESCRIPTION OF THE DRAWINGS

The present disclosure broadly lies in the field of pharmaceutics, particularly, TGF-beta inhibition. Specifically, the present invention relates to TGF-beta inhibition utilizing certain agents such as Artemisinin, antisense oligonucleotides. The present invention also provides the composition comprising the said agents, method of treatment and method of use involving said agents.

FIG. 1: FIG. 1 shows a process flow chart for extraction of Artemisinin.

FIG. 2A: FIG. 2A shows a chart of the time dependent improvement in symptoms for patients treated with ARTIVeda™+SOC versus SOC alone.

FIG. 2B: FIG. 2B shows a chart of the time dependent improvement in symptoms for patients treated with SOC alone.

FIG. 3: FIG. 3 shows a site specific SOC.

FIG. 4: FIG. 4 shows days to reduction of 1 WHO scale i.e. 2 to 1 and 4 to 3. The solid line is SOC+ARTIVeda™ and the dotted line is SOC alone.

FIG. 5: FIG. 5 shows Log-Rank Statistical Analysis for rate of recovery between ARTIVeda™+SOC and SOC alone. ARTIVeda™ is benefiting the patients and the sicker the patients as shown by increasing WHO scale, the more obvious the differences between ARTIVeda™ treated versus ARTIVeda™ untreated.

FIG. 6: FIG. 6 shows a Manufacturing Process Flow Chart for Artemisinin Immediate Release Capsules 500MG.

FIG. 7: FIG. 7 shows OT-101 Treatment Suppressed IL-6.

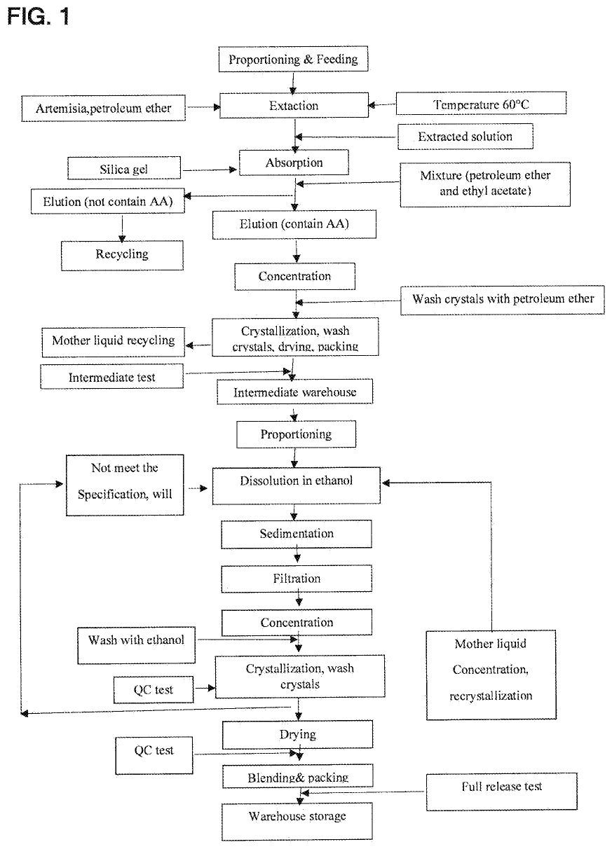

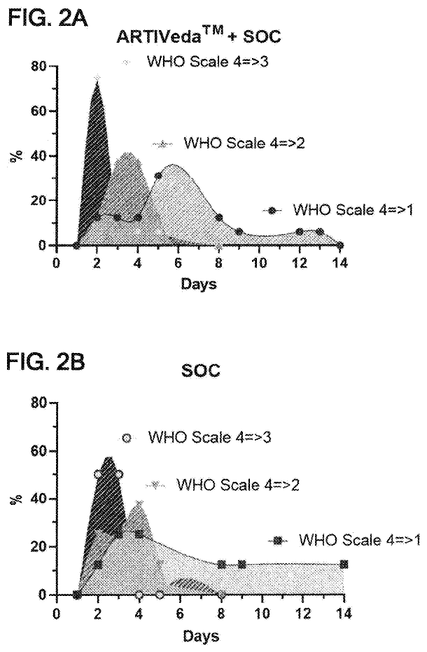

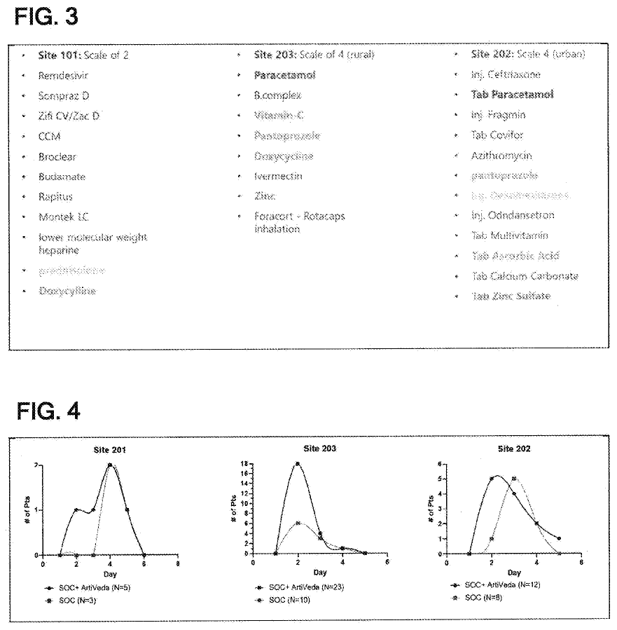

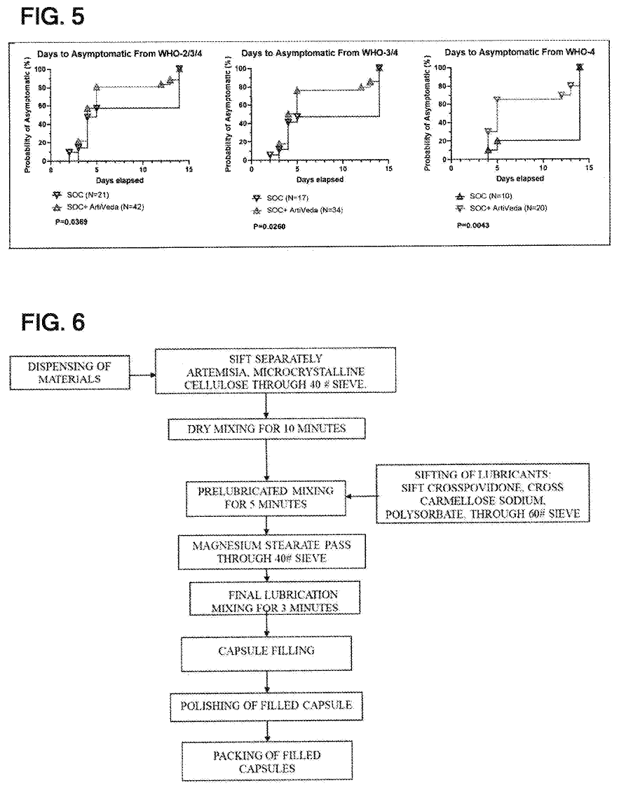

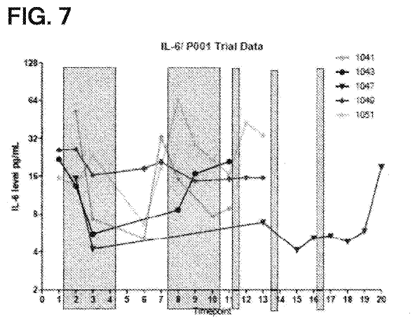

### DETAILED DESCRIPTION

The principal objective of the present invention is to provide TGF-beta inhibition by administering agent selected from the group comprising of Artemisinin, OT-101 antisense oligonucleotide.

One of objective of the present invention is to provide composition comprising the agents selected from the group comprising of Artemisinin, OT-101 antisense oligonucleotide and other anti-sense oligonucleotides.

Yet another objective of the present invention is to provide TGF-beta inhibition by administering Artemisinin.

Yet another objective of the present invention is to provide TGF-beta inhibition by administering OT-101.

Another objective of the present invention is to provide a substantially pure Artemisinin having a purity of more than 90%.

Yet another objective of the present invention is to provide a substantially pure Artemisinin free of the impurity Thujone.

Yet another object of the present invention is to provide a substantially pure Artemisinin with negligible amount of the impurities such as Artemisinin, 9-epiartemisinin.

One more objective of the present invention is to provide Artemisinin for use in the treatment or prophylaxis of viral or pulmonary diseases.

Yet another objective of the present invention is to provide Artemisinin for use in the treatment of COVID-19.

One more objective of the present invention is to provide Artemisinin for use in the treatment of COVID-19.

Yet another objective of the present invention is to provide composition comprising the agents selected from the group comprising of Artemisinin, OT-101 antisense oligonucleotide along with one or more additional therapeutic agents.

Yet another objective of the present invention is to provide a method of treating a fibrosis or any collagen related diseases, cancers, viral diseases, bacterial diseases, fungal diseases, parasite born diseases by administering to a subject agent selected from the group comprising of Artemisinin, OT-101 antisense oligonucleotide and other anti-sense oligonucleotides optionally with one or more additional therapeutic agents.

Yet another objective of the present invention is to provide a method of treating COVID-19 by administering to a subject agent selected from the group comprising of Artemisinin, OT-101 antisense oligonucleotide and other anti-sense oligonucleotides optionally with one or more additional therapeutic agents.

Yet another objective of the present invention is to provide a method of treatment by administering the agents by intravenous, intrathecal, intramuscular, oral, and any other acceptable route of administration.

Yet another objective of the present invention is to provide a pharmaceutically acceptable oral dosage form comprising artemisinin.

Yet another objective of the present invention is to provide a process of extraction of artemisinin.

Yet another objective of the present invention is to provide a composition of matter comprising artemisinin.

Yet another objective of the present invention is to provide a composition of matter of derivatives of artemisinin such as artemether (ARM), artesunate (ARS) and dihydroartemisinin.

Yet another objective of the present invention is to provide Artemisia annua extract comprising Artemisinin, Artemisitene, 9 -epiartemisinin and Thujone.

Yet another objective of the present invention is to provide the composition of matter comprising Artemisinin formulated as drug product.

Yet another objective of the present invention is to provide a composition of matter comprising an anti-sense oligonucleotide OT-101 or OT-101 in combination with anti-sense oligonucleotide sequence selected from SEQ ID NOS:5-12 wherein the backbone is modified as OME or LNA, pharmaceutical composition thereof, and use thereof in treatment of viral diseases including COVID-19.

Yet another objective of the present invention is to provide method of treating TGF-beta storm.

One more objective of the present invention is to provide a method of use of anti-sense oligonucleotide by suppression of TGF-beta induced proteins including IL-6, TGFBIp.

The present disclosure broadly lies in the field of pharmaceutics, particularly, TGF-beta inhibition. Specifically, the present invention relates to TGF-beta inhibition utilizing certain agents such as Artemisinin, antisense oligonucleotides. The present invention also provides the composition comprising the said agents, method of treatment and method of use involving said agents.

At the very outset of the detailed description, it may be understood that the ensuing description only illustrates a particular form of this invention. However, such a particular form is only exemplary embodiment, and without intending to imply any limitation on the scope of this invention. Accordingly, the description is to be understood as an exemplary embodiment and teaching of invention and not intended to be taken restrictively.

Accordingly, an important embodiment of the present invention relates TGF-beta inhibition by administering agent selected from the group comprising of Artemisinin, OT-101 antisense oligonucleotide.

In one aspect of the embodiment, the present invention relates to the TGF-beta inhibition by administering agent selected from the group comprising of Artemisinin, OT-101 antisense oligonucleotide wherein TGF-beta can be TGF-beta1, or TGF-beta2 or TGF-beta3.

In another aspect of the embodiment, the present invention, the present invention relates to the TGF-beta inhibition by administering Artemisinin.

In yet another aspect of the embodiment, the present invention relates to the TGF-beta inhibition by administering anti-sense oligonucleotide, preferably OT-101.

In one more aspect of the embodiment, the present invention relates to the TGF-beta inhibition by administering anti-sense oligonucleotide, preferably OT-101 or OT-101 wherein the backbone is modified as OME or LNA.

In yet another aspect of the embodiment, the present invention relates to TGF-beta inhibition by administering anti-sense oligonucleotide OT-101 or OT-101 in combination with antisense oligonucleotide sequence selected from SEQ ID NOS:5-12.

In one more aspect of embodiment the agents are administered to a human or an animal.

Another embodiment of the present invention relates composition comprising the agents selected from the group comprising of Artemisinin, OT-101 antisense oligonucleotide and other anti-sense oligonucleotides for TGF-beta inhibition.

In one aspect of the embodiment, the present invention relates to a composition comprising the agent Artemisinin for TGF-beta inhibition.

In another aspect of the embodiment, the present invention relates to a composition comprising the agent OT-101 or OT-101 the backbone is modified as OME or LNA, for TGF-beta inhibition. Another important embodiment of the present invention relates to a substantially pure Artemisinin having a purity of more than 90%.

In one aspect of the embodiment the present invention relates to substantially pure Artemisinin free of the impurities such as Thujone.

In another aspect of the embodiment the present invention relates to substantially pure Artemisinin negligible amount of the impurities such as Artemisinin, 9-epiartemisinin.

In yet another aspect of the embodiment the present invention relates to substantially pure Artemisinin free of the impurities such as Artemisinin, 9-epiartemisinin and Thujone.

In one of the embodiment the present invention relates to a process of extraction of artemisinin, from the plant Artemisia annua comprising the steps of extracting the plant extract with water, partitioning the extract between water and petroleum ether, chromatographing the extracted solution on silica gel adsorbent with a solvent comprising petroleum ether and ethyl acetate to obtain artemisinin in eluted solution and evaporating the eluted solution to obtain oily material followed by crystallization to produce substantially pure artemisinin.

In one more embodiment the present invention relates to Artemisinin for use in the treatment or prophylaxis of viral or pulmonary diseases.

In one more aspect of the embodiment the present invention relates to Artemisinin for use in the treatment or prophylaxis of viral diseases including but not limited to SARS, MERS, RSV, Coronavirus, HIV, Ebola, Cytomegalovirus (CMV). Human herpes virus type 6 (HHV-6), Herpes simplex virus (HSV-1 and HSV2), Epstein-Barr virus (EBV), Hepatitis B virus (HBV).

In another aspect of the embodiment the present invention relates to Artemisinin for use in the treatment or prophylaxis of viral disease such as COVID-19.

In one of the embodiment the present invention relates to a pharmaceutical composition comprising Artemisinin in free, or pharmaceutically acceptable salts form, polymorphs or stereoisomers or mixtures thereof, optionally along with pharmaceutically acceptable excipients.

In one aspect of the embodiment the present invention relates to the composition Artemisinin, stabilizers selected from polysobate 80 and polysorbate 80 dry powder, diluents selected from microcrystalline cellulose, disintegrants selected from crospovidone and croscarmellose and anticaking agent selected from magnesium stearate.

In another aspect of the embodiment the present invention provides the pharmaceutical composition wherein the composition comprises 88-97 weight % of Artemisinin, 1-5 weight % of stabilizers, 0.2-1 weight % of diluents, 1-4 weight % of disintegrants and 1-2 weight % of anticaking agents.

In another aspect of the embodiment the present invention provides a pharmaceutical composition comprising Artemisinin in free, or pharmaceutically acceptable salts form, polymorphs or stereoisomers or mixtures thereof and one or more pharmaceutically acceptable excipient selected from the group consisting of diluents, stabilizers, disintegrants and anticaking agent, wherein composition comprises 45-99% w/w of Artemisinin, 1-50% w/w of diluents and 2-20% w/w anticaking agent.

In another aspect of the embodiment the present invention provides the pharmaceutical composition comprising substantially pure Artemisinin having a purity of more than 90%.

In another aspect of the embodiment the present invention provides the pharmaceutical composition comprising substantially pure Artemisinin free from Artemisinin, 9-epiartemisinin and Thujone impurities.

In yet another embodiment the present invention provides a method of treating a fibrosis or any collagen related diseases, cancers, viral diseases, bacterial diseases, fungal diseases, parasite born diseases wherein the method comprises administering to a subject a therapeutically effective amount of Artemisinin.

In one aspect of the embodiment the method is for treating viral disease induced by, but not limited to SARS, MERS, RSV, coronavirus, HIV, Ebola, Cytomegalovirus (CMV). Human herpes virus type 6 (HHV-6), Herpes simplex virus (HSV-1 and HSV2), Epstein-Barr virus (EBV), Hepatitis B virus (HBV).

In one more aspect of the embodiment the present invention relates a method of treating COVID-19 administering to a subject a therapeutically effective amount of Artemisinin.

In one more aspect of the embodiment the Artemisinin inhibits TGF-beta, wherein TGF-beta is TGF-beta1, or TGF-beta2 or TGF-beta3.

In one more aspect the administration includes intravenous, intrathecal, intramuscular, oral, and any other acceptable route of administration. In yet another embodiment, the present invention relates to Artemisinin for use in the treatment of COVID-19.

Another important embodiment of the present invention relates to provides composition comprising the Artemisinin.

Another important embodiment of the present invention relates to a pharmaceutically acceptable oral dosage form comprising artemisinin.

In one aspect of the embodiment the present invention relates a pharmaceutically acceptable oral dosage form comprising artemisinin in an amount of 250-750 mg each day for five days, preferably in an amount 500 mg each day for five days.

Efficacy of ARTIVeda™ (Artemisinin/Artemisia absinthium plant extract/Damanaka per Ayurvedic text)—Artemisia extract—was found to have activity against COVID-19 based on our own internal studies (clinical and cell based) with independent confirmation from others across the globe. The data is strongly supportive of ARTIVeda™ as therapeutic against COVID-19.

Bioactives in plants, such as Artemesia, are secondary metabolites that are intimately involved in the cellular metabolism and plant physiology that are created to further improve survival of plant as part of the coevolution of plant within the ecosystem as defense against pathogens such as viruses, as attractor for pollinators such as insects, and the health of the disseminators such as grazing animals. What started out as pharmacophore to confer survival advantage is exploited by human to treat maladies afflicted them. Over thousands of years, the traditional herbal medicine is codified into various system of traditional medicines. Ethnobiology take insights garnered from traditional medicine information for the development of pharmaceutical drug.

Artemisia species are widely use in traditional medicine. Artemisia are mostly herb, and sometimes shrubs, usually with strong aroma. Plant bodies are often densely hairy. Leaves are pinnatifid to pinnatisect with variable dimensions. Capitulum inflorescence is generally in the form of a paniculate-raceme arrangement. Herbaceous involucral bracts are present. Receptacles are convex or flat and naked or covered by hairs. Ray florets are pistillate. Corolla color is yellow or green and rarely brown. Disk florets are bisexual. Cypselas are obovoid to oblong and mostly brown. There are three well known species that are in cultivation in India. Artemisia annua, though not indigenous to India, is now cultivated widely in Kashmir valleys, hills of Himachal Pradesh, Uttar Pradesh, and other parts of the country. The chemical composition of Artemisia consists of volatile and nonvolatile constituents, mainly sesquiterpenoids, including artemisinin.
1. A. absinthium L. (Vilayati afsantin, Afsantin, Kakamush, Afsantheen, Zoon). Ethnobotanical uses: 1. The dried plant is used to protect clothes against insects and as an insecticide. 2. The whole plant decoction is used as a tonic for general health. 3. Leaf powder is used for gastric problems and intestinal worms. 4. Seed powder is taken orally to treat rheumatism. 5. Seed powder paste is applied on teeth for pain relief. Trade name: Dvipantara Damanaka.
2. A. annua L. (Afsantin, Afsantin jari). Ethnobotanical uses: 1. A decoction of the whole plant is used for treatment of Malaria. 2. Leaves are used for fever, cough and common cold. 3. Dry powder of leaves is taken to treat diarrhea. 4. Oil of afsantin is used in local perfumes (ettar) due to its pleasant fragrance. Trade name: Seeme Davana.
3. A. vulgaris L. (Tatwan, Nagdowna, Tarkha). Ethnobotanical uses: 1. A leaf infusion is used in fever. 2. The tomentum is used as moxa. Trade name: Dvipantara Damanaka.

### Pharmaceutical Vegetable Capsule Compositions Comprising Artemisinin

In one more embodiment the present disclosure relates to pharmaceutical vegetable capsules comprising artemisinin, in free, or pharmaceutically acceptable salts form, polymorphs or stereoisomers or mixtures thereof, optionally in combination with one or more additional therapeutic agents, processes or manufacture thereof and methods of use in the treatment or prophylaxis of COVID-19 disease.

In another embodiment, a pharmaceutically acceptable dosage form for pulmonary health support is provided. The pharmaceutically acceptable oral dosage can include a therapeutically effective amount of artemisinin and a pharmaceutically acceptable carrier. The oral dosage form can, when measured using a USP Type-II dissolution apparatus in 900 mL of sodium phosphate buffer of pH 6.8 with 2% (w/v) sodium lauryl sulfate at 75 rpm at 37° C., releases at least 70 wt % of artemisinin after 45 minutes, or in the alternative release at least 20 wt % more after 45 minutes than an equivalently dosed oral dosage form without the carrier.

In another embodiment, the pharmaceutical composition of the present invention of vegetable capsule of oral dosage form can be packaged in HDPE bottles or blister packs.

### Artemisinin Dosing: Selection of 500 Mg Oral Dose Each Day for Five Days as the Optimal Dose.

The pharmacokinetics of artemisinin was studied in multiple clinical trials previously for malaria at the various dose level. By analyzing the data from those clinical trials we arrived at the optimal dose of 500 mg dose level once a day for five days follow by 5 days break. This completed one cycle of treatment and patients are allowed to continue up to 3 cycles of treatment as necessary to achieve complete recovery.

Another embodiment the present invention relates to a composition comprising the Artemisinin along with one or more additional therapeutic agents.

In one more aspect of the embodiment the present invention provides a pharmaceutical composition Artemisinin in free, or pharmaceutically acceptable salts form, polymorphs or stereoisomers or mixtures thereof, further comprising one or more additional therapeutic agents.

In one of the aspect one or more additional therapeutic agent is selected from Piperiquine, Pyronaridine, Curcumin, Frankincense, or SOC.

In another aspect of the embodiment, SOC is defined as the treatment with the drugs selected from Remdesivir, Sompraz D, Zifi CV/Zac D, CCM, Broclear, Budamate, Rapitus, Montek LC, lower molecular weight heparine, prednisolone, Doxycylline Paracetamol, B. complex, Vitamin-C, Pantoprozol, Doxycycline, Ivermectin, Zinc, Foracort-Rotacaps inhalation, Injection Ceftriaxone, Tab Paracetamol, Injection Fragmin, Tablet Covifor, Azithromycin, pantoprazole, Injection Dexamethasone, Injection Odndansetron, Tablet Multivitamin, Tablet Ascorbic Acid, Tablet Calcium Carbonate, Tablet Zinc Sulfate.

In one more aspect of the embodiment, the present invention relates to a pharmaceutical composition comprising Artemisinin, Curcumin, Frankincense, and vitamin C.

In one more aspect of the embodiment, the present invention relates to a pharmaceutical composition comprising Artemisinin and piperaquine.

In another aspect of the embodiment, the present invention relates to a pharmaceutical composition comprising Artemisinin and pyronaridine in 70:30 to 30:70 weight %.

In yet another aspect of the embodiment, the composition is in form of a nanoparticular formulation.

In yet another aspect of the embodiment, the composition is in form of a spray.

In one of the aspects the present invention provides a composition comprising Artemisinin along with Curcumin. The product ArtemiC is a medical spray comprised of Artemisinin Curcumin, Frankincense and vitamin C. ArtemiC demonstrates the following distinct advantages:
1. A full safety and efficacy profile with no drug-adverse events;
2. The ability to prevent deterioration of COVID-19 patients and achieve faster clinical improvement;
3. The ability to assist in reducing the pressure on the medical system and support coping with hospitalised patients;
4. The ability to improve symptoms and pain associated with COVID-19;
5. The versatility to be used in community as well as in hospitals; and
6. As the mechanism of action of ArtemiCTM is focused on the anti-inflammatory effect and prevention of cytokine storm, a wide spectrum of potential indications will be considered for future development.

In one more aspect of the embodiment, the present invention provides a preparation of ArtemiC, comprising Artemisinin, Curcumin, Boswellia, and Vitamin C in a nanoparticular formulation, is proposed as a treatment for the disease associated with the novel corona virus SARS-CoV-2. It is readily available in light of its status as a food supplement. This initiative is presented under the urgent circumstances of the fulminant pandemic caused by this lethal disease, which is known as COVID-19 and has spread across the globe causing death and disrupting the normal function of modern society. The grounds for the proposal are rooted in existing knowledge on the components and pharmacological features of this formulation and their relevance to the current understanding of the disease process being addressed.

Leading among these considerations are well established immuno-modulatory activities of the active ingredients as established in vitro and in vivo and published over the years. These activities as apparent, for example, in diminishing activity of TNF alpha and IL-6 levels are acknowledged to be relevant to the pathophysiology processes involved in the progressive form of COVID-19. The active agents have in addition prominent anti-oxidant, anti-inflammatory as well as anti-aggregant and anti-microbial activities.

Based on these activities and observations in animal models, together with clinical experience of the separate ingredients and in various combinations in other contexts it is proposed to evaluate their effect in the context of COVID-19.

In one of the embodiments the present invention relates a Composition of matter comprising artemisinin.

In one embodiment the present invention relates a composition of matter of derivatives of artemether (ARM), artesunate (ARS) and dihydroartemisinin. artemisinin such as.

In one embodiment the present invention relates a Artemisia annua extract comprising Artemisinin, Artemisitene, 9-epiartemisinin and Thujone.

In one aspect of the embodiment the present invention relates a composition of matter formulated as drug product.

In one more aspect of the embodiment the drug product is capsules, tablets, powders, pouches, sachets, suppository.

In yet another aspect of the embodiment the drug product is encapsulated in vegetable, hard gelatin or soft gelatin capsules.

In another aspect of the embodiment the present invention relates to drug product formulated as capsules, tablets, powders, pouches, sachets, suppository for release of the drug immediate release, sustained release or modified release.

In yet another aspect of the embodiment dissolution profile is such that greater 40% dissolution is achieved within 15 min.

Another important embodiment of the present invention relates to a composition of matter comprising an anti-sense oligonucleotide OT-101 or OT-101 in combination with anti-sense oligonucleotide sequence selected from SEQ ID NOS:5-12 wherein the backbone is modified as OME or LNA, pharmaceutical composition thereof, and use thereof in treatment of viral diseases including COVID-19.

Anti-sense oligonucleotides of this invention include the following:

SEQ ID NO: 5,
gtaggtaaaa acctaatat.
SEQ ID NO: 6,
gttcgtttag agaacagatc.
SEQ ID NO: 7,
taaagttcgt ttagagaaca g.
SEQ ID NO: 8,
agccctgtat acgac.
SEQ ID NO: 9,
gtaggtaaaa acctaatat.
SEQ ID NO: 10,
cgtttagaga acagatctac.
SEQ ID NO: 11,
cattgtagat gtcaaaagcc.
SEQ ID NO: 12,
ctccctcatg gtggcagttg a.
SEQ ID NO: 13,
cggcatgtct attttgta. (OT-101)

Antisense oligonucleotides given in SEQ ID NOs:5-13 herein can be chemically-modified, as known in the art.

Antisense oligonucleotide OT-101 is SEQ ID NO:13.

Another important embodiment of the present invention relates to a pharmaceutical composition comprising antisense oligonucleotide selected from sequences OT-101, any of SEQ ID NOS:5-12, or a combination thereof, optionally along with one or more pharmaceutically acceptable excipients.

In one of the aspects one or more pharmaceutically acceptable excipients is selected from the group comprising of vehicles, stabilizers, diluents, disintegrants, anticaking agents and/or additives.

In one more aspect of the embodiment the present invention relates to a pharmaceutical composition comprising OT-101 in combination with any of SEQ ID NOS:5-12 in ratio of 1:1 to 1:100.

In one more aspect of the embodiment the present invention relates to a pharmaceutical composition comprising OT-101 in combination with any of SEQ ID NOS:5-12 wherein the backbone is modified as OME or LNA, further comprising one or more additional therapeutic agents.

In yet another aspect of the embodiment the composition is in form of a nanoparticular formulation.

### Design of ASO Against SARS-CoV2

Success or failure of an anti-sense experiment fundamentally depends on first selecting the right target sequence within the particular mRNA of interest. The antisense oligonucleotide (ASO), along with its appropriate chemical modifications, is then designed around that sequence. The following should be taken into consideration when selecting the mRNA target sequence:

### ASO Length.

The ASO comprises at least 8 nucleotides, optimally 20 nucleotides. The ratio of residues forming 3 hydrogen bonds and 2 hydrogen bonds should be >=2.9.

ASO should be about 20 bases long; such oligos are easy to synthesize, form stable DNA-RNA duplexes, and are long enough to be unique, at least in the human genome. Uniqueness is important; it is critical that the ASO does not bind, even partially, to a non-target mRNA. If as few as 6-7 base pairs are formed between the ASO and non-target mRNA, that likely would be sufficient to initiate RNase H activity, leading to cleavage of the wrong target.

Blast search to identify mRNA with 100% match was negative for the entire sequence; 100% matches for the partial sequence were found — except TRS-1, the Tₘ is not above 37° C. to be of concern. Another sequence, the FS, exhibited abnormally short sequence and low Tₘ and therefore would not be suitable as therapeutic antisense.

The following ASOs are being evaluated against COVID-19.

| | | | | | | Off Target |
|---|---|---|---|---|---|---|
| Name | Sequence (5′-3′) | | | | | mRNA |
| 5TERM | GGTAGGTAAAAACCTAATAT (SEQ ID NO: 14) | | | | | Highest |
| (1-20) | | | | | | match = 14 nt/Tₘ of |
| | | | | | | 25.0 |
| | Homo sapiens protein geranylgeranyltransferase type I subunit | | | | | 14 nt/25.0° C. |
| | beta (PGGT1B), mRNA | | | | | |
| | Sequence ID: NM_005023.4Length: 9550Number of Matches: 1 | | | | | |
| | Alignment statistics for match #1 | | | | | |
| | Score | Expect | Identities | Gaps | Strand | |
| | 28.2 bits(14) | 12 | 14/14(100%) | 0/14(0%) | Plus/Plus | |
| | Query 7 TAAAAACCTAATAT 20 (SEQ ID NO: 15) | | | | | |
| | Sbjct 4678 TAAAAACCTAATAT 4691 (SEQ ID NO: 16) | | | | | |
| | Homo sapiens CA5BP1-CA5B readthrough (CA5BP1-CA5B), | | | | | 14 nt/27.0° C. |
| | transcript variant 1, long non-coding RNA | | | | | |
| | Sequence ID: NR_160544.1 Length: 8116Number of Matches: 1 | | | | | |
| | Alignment statistics for match #1 | | | | | |
| | Score | Expect | Identities | Gaps | Strand | |
| | 28.2 bits(14) | 12 | 14/14(100%) | 0/14(0%) | Plus/Plus | |
| | Query 6 GTAAAAACCTAATA 19 (SEQ ID NO: 17) | | | | | |
| | Sbjct 1271 GTAAAAACCTAATA 1284 (SEQ ID NO: 18) | | | | | |
| | Homo sapiens integrator complex subunit 14 (INTS14), | | | | | 13 nt/27.0° C. |
| | transcript variant 3, non-coding RNA | | | | | |
| | Sequence ID: NR_045105.3Length: 2678Number of Matches: 1 | | | | | |
| | Alignment statistics for match #1 | | | | | |
| | Score | Expect | Identities | Gaps | Strand | |
| | 26.3 bits(13) | 49 | 13/13(100%) | 0/13(0%) | Plus/Plus | |
| | Query 3 TAGGTAAAAACCT 15 (SEQ ID NO: 19) | | | | | |
| | Sbjct 2099 TAGGTAAAAACCT 2111 (SEQ ID NO: 20) | | | | | |
| TRS1 | GTTCGTTTAGAGAACAGATC (SEQ ID NO: 21) | | | | | Highest |
| (53-72) | | | | | | match = 14 nt/Tₘ of |
| | | | | | | 31.0° C. |
| | PREDICTED: Homo sapiens uncharacterized LOC105375623 | | | | | 14 nt/31.0° C. |
| | (LOC105375623), transcript variant X1, ncRNA | | | | | |
| | Sequence ID: XR_928373.3Length: 743Number of Matches: 1 | | | | | |
| | Alignment statistics for match #1 | | | | | |
| | Score | Expect | Identities | Gaps | Strand | |
| | 28.2 bits(14) | 12 | 14/14(100%) | 0/14(0%) | Plus/Plus | |
| | Query 5 GTTTAGAGAACAGA 18 (SEQ ID NO: 22) | | | | | |
| | Sbjct 296 GTTTAGAGAACAGA 309 (SEQ ID NO: 23) | | | | | |
| | Homo sapiens plakophilin 4 (PKP4), transcript variant 8, mRNA | | | | | 13 nt/29.0° C. |
| | Sequence ID: NM_001377220.1Length: 5842Number of | | | | | |
| | Matches: 1 | | | | | |
| | Range 1: 1292 to 1304GenBankGraphicsNext Match Previous | | | | | |
| | Match | | | | | |
| | Alignment statistics for match #1 | | | | | |
| | Score | Expect | Identities | Gaps | Strand | |
| | 26.3 bits(13) | 49 | 13/13(100%) | 0/13(0%) | Plus/Plus | |
| | Query 5 GTTTAGAGAACAG 17 (SEQ ID NO: 24) | | | | | |
| | Sbjct 1292 GTTTAGAGAACAG 1304 (SEQ ID NO: 25) | | | | | |
| | Homo sapiens golgin, RAB6 interacting (GORAB), transcript | | | | | 13 nt/29.0° C. |
| | variant 1, mRNA | | | | | |
| | Sequence ID: NM_152281.3Length: 2540Number of Matches: 1 | | | | | |
| | Alignment statistics for match #1 | | | | | |
| | Score | Expect | Identities | Gaps | Strand | |
| | 26.3 bits(13) | 49 | 13/13(100%) | 0/13(0%) | Plus/Plus | |
| | Query 1 GTCGTTTAGAGA 13 (SEQ ID NO: 26) | | | | | |
| | Sbjct 971 GTTCGTTTAGAGA 983 (SEQ ID NO: 27) | | | | | |
| TRS2 | TAAAGTTCGTTTAGAGAACAG (SEQ ID NO: 28) | | | | | Highest |
| (56-76) | | | | | | match = 13 nt/Tₘ of |
| | | | | | | 29.0 |
| | Homo sapiens plakophilin 4 (PKP4), transcript variant 8, mRNA | | | | | 13 nt/29.0° C. |
| | Sequence ID: NM_001377220.1Length: 5842Number of | | | | | |
| | Matches: 1 | | | | | |
| | Range 1: 1292 to 1304GenBankGraphicsNext Match Previous | | | | | |
| | Match | | | | | |
| | Alignment statistics for match #1 | | | | | |
| | Score | Expect | Identities | Gaps | Strand | |
| | 26.3 bits(13) | 61 | 13/13(100%) | 0/13(0%) | Plus/Plus | |
| | Query 9 GTTTAGAGAACAG 21 (SEQ ID NO: 29) | | | | | |
| | Sbjct 1292 GTTTAGAGAACAG 1304 (SEQ ID NO: 30) | | | | | |
| | Homo sapiens golgin, RAB6 interacting (GORAB), transcript | | | | | |
| | variant 1, mRNA | | | | | |
| | Sequence ID: NM_152281.3Length: 2540Number of Matches: 1 | | | | | |
| | Alignment statistics for match #1 | | | | | |
| | Score | Expect | Identities | Gaps | Strand | |
| | 26.3 bits(13) | 61 | 13/13(100%) | 0/13(0%) | Plus/Plus | |
| | Query 5 GTCGTTAGAGA 11 (SEQ ID NO: 31) | | | | | |
| | Sbjct 971 GTTCGTTTAGAGA 983 (SEQ ID NO: 32) | | | | | |
| | Homo sapiens zinc finger protein 57 (ZNF57), transcript variant | | | | | |
| | 1, mRNA | | | | | |
| | Sequence ID: NM_173480.3Length: 1970Number of Matches: 2 | | | | | |
| | Alignment statistics for match #1 | | | | | |
| | Score | Expect | Identities | Gaps | Strand | |
| | 26.3 bits(13) | 61 | 13/13(100%) | 0/13(0%) | Plus/Plus | |
| | Query 8 CGTTTAGAGAACA 20 (SEQ ID NO: 33) | | | | | |
| | Sbjct 1251 CGTTTAGAGAACA 1263 (SEQ ID NO: 34) | | | | | |
| | Range 2: 1419 to 1431GenBankGraphicsNext Match Previous | | | | | |
| | Match First Match | | | | | |
| | Alignment statistics for match #2 | | | | | |
| | Score | Expect | Identities | Gaps | Strand | |
| | 26.3 bits(13) | 61 | 13/13(100%) | 0/13(0%) | Plus/Plus | |
| | Query 8 CGTTTAGAGAACA 20 (SEQ ID NO: 35) | | | | | |
| | Sbjct 1419 CGTTTAGAGAACA 1431 (SEQ ID NO: 36) | | | | | |
| FS | AGCCCTGTATACGAC (SEQ ID NO: 37) | | | | | match = 13 nt/Tₘ of |
| (13,458- | | | | | | 33.0 |
| 13,472) | | | | | | |
| | Homo sapiens BAI1 associated protein 3 (BAIAP3), transcript | | | | | 13 nt/33.0° C. |
| | variant 6, mRNA | | | | | |
| | Sequence ID: NM_001286464.2Length: 4582Number of | | | | | |
| | Matches: 1 | | | | | |
| | Alignment statistics for match #1 | | | | | |
| | Score | Expect | Identities | Gaps | Strand | |
| | 26.3 bits(13) | 24 | 13/13(100%) | 0/13(0%) | Plus/Plus | |
| | Query 3 CCCTGTATACGAC 15 (SEQ ID NO: 38) | | | | | |
| | Sbjct 3289 CCCTGTATACGAC 3301 (SEQ ID NO: 39) | | | | | |
| | Homo sapiens osteoclast stimulating factor 1 (OSTF1), mRNA | | | | | 13 nt/33.0° C. |
| | Sequence ID: NM_012383.5Length: 1348Number of Matches: 1 | | | | | |
| | Alignment statistics for match #1 | | | | | |
| | Score | Expect | Identities | Gaps | Strand | |
| | 26.3 bits(13) | 24 | 13/13(100%) | 0/13(0%) | Plus/Plus | |
| | Query 1 AGCCCTGTATACG 13 (SEQ ID NO:4 0) | | | | | |
| | Sbjct 233 AGCCCTGTATACG 245 (SEQ ID NO: 41) | | | | | |
| | Homo sapiens ubiquitin specific peptidase 18 (USP18), mRNA | | | | | 13nt/33.0° C. |
| | Sequence ID: NM_017414.4Length: 1858Number of Matches: 1 | | | | | |
| | Alignment statistics for match #1 | | | | | |
| | Score | Expect | Identities | Gaps | Strand | |
| | 26.3 bits(13) | 24 | 13/13(100%) | 0/13(0%) | Plus/Plus | |
| | Query 2 GCCCTGTATACGA 14 (SEQ ID NO: 42) | | | | | |
| | Sbjct 655 GCCCTGTATACGA 667 (SEQ ID NO: 43) | | | | | |
| TRS2-2 | CGTTTAGAGAACAGATCTAC (SEQ ID NO: 44) | | | | | Highest |
| (53-72) | | | | | | match = 15 nt/Tₘ of |
| | | | | | | 35.5 |
| | PREDICTED: Homo sapiens uncharacterized LOC105378374 | | | | | 15 nt/35.5^{O} C. |
| | (LOC105378374), ncRNA | | | | | |
| | Sequence ID: XR_002957084.1 Length:12122Number of | | | | | |
| | Matches: 1 | | | | | |
| | Alignment statistics for match #1 | | | | | |
| | Score | Expect | Identities | Gaps | Strand | |
| | 30.2 bits(15) | 3.1 | 15/15(100%) | 0/15(0%) | Plus/Minus | |
| | Query 2 GTTTAGAGAACAGAT 16 (SEQ ID NO: 45) | | | | | |
| | Sbjct 4057 GTTTAGAGAACAGAT 4043 (SEQ ID NO: 46) | | | | | |
| | Homo sapiens DGUOK antisense RNA 1 (DGUOK-AS1), | | | | | 14 nt/31.0° C. |
| | transcript variant 1, long non-coding RNA | | | | | |
| | Sequence ID: NR_104029.1 Length: 582Number of Matches: 1 | | | | | |
| | Alignment statistics for match #1 | | | | | |
| | Score | Expect | Identities | Gaps | Strand | |
| | 28.2 bits(14) | 12 | 14/14(100%) | 0/14(0%) | Plus/Plus | |
| | Query 6 AGAGAACAGATCTA 19 (SEQ ID NO: 47) | | | | | |
| | Sbjct 477 AGAGAACAGATCTA 490 (SEQ ID NO: 48) | | | | | |
| | Homo sapiens plakophilin 4 (PKP4), transcript variant 8, mRNA | | | | | 13 nt/29.0° C. |
| | Sequence ID: NM_001377220.1Length: 5842Number of | | | | | |
| | Matches: 1 | | | | | |
| | Range 1: 1292 to 1304GenB ankGraphicsNext Match Previous | | | | | |
| | Match | | | | | |
| | Alignment statistics for match #1 | | | | | |
| | Score | Expect | Identities | Gaps | Strand | |
| | 26.3 bits(13) | 49 | 13/13(100%) | 0/13(0%) | Plus/Plus | |
| | Query 2 GTTTAGAGAACAG 14 (SEQ ID NO: 49) | | | | | |
| | Sbjct 1292 GTTTAGAGAACAG 1304 (SEQ ID NO: 50) | | | | | |
| FS-2a | CATTGTAGATGTCAAAAGCC (SEQ ID NO: 51) | | | | | Max 15 nt/38.9^{O} C. |
| (13539- | PREDICTED: Homo sapiens uncharacterized LOC101927182 | | | | | 15 nt/38.9^{O} C. |
| 13558) | (LOC101927182), transcript variant X7, ncRNA | | | | | |
| | Sequence ID: XR_001754612.1 Length:1663Number of | | | | | |
| | Matches: 1 | | | | | |
| | Alignment statistics for match #1 | | | | | |
| | Score | Expect | Identities | Gaps | Strand | |
| | 30.2 bits(15) | 3.1 | 15/15(100%) | 0/15(0%) | Plus/Plus | |
| | Query 4 TGTAGATGTCAAAAG 18 (SEQ ID NO: 52) | | | | | |
| | Sbjct 931 TGTAGATTGTCAAAAG 945 (SEQ ID NO: 53) | | | | | |
| | Homo sapiens ryanodine receptor 3 (RYR3), transcript variant | | | | | |
| | 1, mRNA | | | | | |
| | Sequence ID: NM_001036.6Length: 15568Number of Matches: | | | | | |
| | 1 | | | | | |
| | Range 1: 10080 to 10093GenB ankGraphics Next Match | | | | | |
| | Previous Match | | | | | |
| | Alignment statistics for match #1 | | | | | |
| | Score | Expect | Identities | Gaps | Strand | |
| | 28.2 bits(14) | 12 | 14/14(100%) | 0/14(0%) | Plus/Plus | |
| | Query 7 AGATGCCAAAAGCC 20 (SEQ ID NO: 54) | | | | | |
| | Sbjct 10080 AGATCTCAAAAGCC 10093 (SEQ ID NO: 55) | | | | | |
| | Homo sapiens zinc finger protein 600 (ZNF600), transcript | | | | | |
| | variant 2, mRNA | | | | | |
| | Sequence ID: NM_001321866.2Length: 4618Number of | | | | | |
| | Matches: 1 | | | | | |
| | Alignment statistics for match #1 | | | | | |
| | Score | Expect | Identities | Gaps | Strand | |
| | 28.2 bits(14) | 12 | 14/14(100%) | 0/14(0%) | Plus/Plus | |

| | |
|---|---|
| | Query 1 CATTGTAGATGTCA 14 (SEQ ID NO: 56) |
| | Sbjct 1668 CATTGTAGATGTCA 1681 (SEQ ID NO: 58) |
| RSV1 | CTCCCTCATGGTGGCAGTTGA (SEQ ID NO: 58) |

Presence of CG Motifs in Target mRNA/ASO

Because unmethylated CG motifs are common in bacterial, but not eukaryotic, DNA, their presence in an anti-sense oligo may trigger an immune response in in vivo experiments if the organism's immune system interprets it as a bacterial infection. CG-mediated immune response is particularly strong when the CG sequence is embedded as part of a purine-purine-C-G-pyrimidine-pyrimidine sequence. One way to avoid this problem is to be careful to choose oligos that either lack CG, or at least lack the above flanking sequences around a CG. If elimination of CG is not possible, then a good alternative is to replace the C in CG with 5-methyl-C, which does not stimulate the immune system or deleteriously affect hybridization.

Oligonucleotides containing CG can act as immunostimulators by causing proliferation of B lymphocytes; by activating macrophages, dendritic cells, and T cells; and by inducing cytokine release. These CG-mediated immune effects depend on the sequences flanking the CG dimer, and are strongest with the purine.purine.CG.pyrimidine.pyrimidine motif. These CG effects occur with phosphorothioates as well as with phosphodiesters, and may be responsible for some of the activities of oligonucleotides reported in vivo.

Formation of Tetraplexes within ASO

ASOs should not contain 4 or more consecutive elements/nucleotides (CCCC or GGGG). Furthermore, ASOs should not contain 2 or more series of 3 consecutive elements/nucleotides (CCC or GGG).

ASOs containing either single GGGG runs or repeated GG or GGG runs in close proximity can form intra-strand tetraplexes (single structures of four strands). G tetraplexes often have high affinity for proteins, which can result in potent, non-antisense biological effects that may interfere with an anti-sense experiment, particularly when such effects mimic anti-sense activity. Whenever possible, such G motifs should be avoided. When elimination of such motifs is unavoidable, then a good alternative is to replace one or more of the Gs with 7-deaza-G or 6-thio-G, which block G-tetraplex formation.

Formation of tetraplexes with potent biological activity has caused some problems in the antisense field. Investigators should carefully examine all oligonucleotides very rich in a particular nucleoside, particularly if they show repeated sequences or have multiple occurrences of two or more adjacent identical bases. Oligomers with multiple repeats of two or more consecutive Gs or Cs may form tetraplexes and other non-Watson-Crick structures. Not all oligomers with such features will necessarily form these higher order structures, particularly in physiological conditions. Nonetheless, such sequences raise warning flags and there is a well-documented danger in ascribing biological effects to an antisense mechanism without careful investigation.

The most extensively studied tetraplexes are formed by oligonucleotides containing multiple adjacent guanine residues. These may occur in a single run of around four residues but they can also be found in repeated GG or GGG motifs that occur in close proximity. Even if they do not form tetraplexes, G-rich sequences with multiple GG dimers may form other unusual structures depending on sequence context. Tetraplex-forming runs of Gs seem to have an affinity for various proteins and when included in synthetic oligonucleotides, they produce a multitude of biological effects. For example, researchers have identified tetraplexes that bind to thrombin and to the HIV envelope protein. Other tetraplexes have been shown to bind to transcription factors or to produce antiproliferative effects by protein binding. The ability to form tetraplexes can be blocked by replacing guanosine residues with 7-deazaG or 6-thioG . It should also be noted that a phosphorothioate oligonucleotide containing only C residues was shown to have activity similar to one containing a G-tetraplex.

### Anti-Sense Activity-Increasing/Decreasing Motifs

Several studies have conclusively shown that the activity of an ASO against its mRNA target is sequence-motif content-dependent. A major study of over 1000 phosphorothiolated ASOs showed that the presence of motifs CCAC, TCCC, ACTC, GCCA, and CTCT positively correlated with anti-sense activity, while GGGG, ACTG, TAA, CCGG, and AAA negatively correlated with anti-sense activity.

Investigators have suggested that stretches of purines in the target might stabilize the heteroduplex formed. From examining the sequences of active antisense oligonucleotides in many published studies, investigators have proposed that selecting a target containing the sequence GGGA gives a much better chance of success.

### Conformational and Thermodynamic Considerations

The major problem lies with the secondary and tertiary folding that can make much of the RNA inaccessible to a molecule as large as an oligonucleotide. Even those sequences that appear to be accessible may already be involved in intramolecular hydrogen bonding, stacking interactions, or in solvation that would be disrupted by hybridization of an oligonucleotide. Consequently, hybridization-induced rearrangement of the existing RNA structure may carry a prohibitive thermodynamic penalty. On the other hand, single-stranded sequences within the RNA may be preordered by stacking into helical conformations that are particularly favorable for hybridization. The exceptional stability of hybrids formed between the loops of two hairpins (kissing interactions) is well known and is important in the association of natural antisense RNAs with their targets. Even though the rules for base-pairing are very simple, additional subtleties govern the hybridization of oligonucleotides to RNA that are not well understood. The behavior of oligonucleotides is very dependent on the terminal nucleotides. Moreover, small changes in the length or a shift in binding site of one or two nucleotides can profoundly affect the kinetics of hybrid formation. Even a few base changes that do not change the thermodynamic stability of the duplex may greatly change the kinetics of hybridization. These effects may partially account for the efficacy of different antisense oligonucleotides in vivo.

### Phosphorothioates

The easy-to-synthesize phosphorothioate oligonucleotides assume the native Watson-Crick nucleotide hydrogen-bonding patterns, can activate RNase H-mediated degradation of cellular mRNA, and are nuclease-resistant. The antisense effects of the phosphorothioates can be observed for over 48 hours after a single application to tissue culture cells. This degree of stability is needed for in vivo work. However, the actual stability of a phosphoro-thioate oligonucleotide in a specific experiment can vary with each sequence and cell line examined. Although early work using these compounds was very encouraging, it has become clear that some of the most exciting results were actually due to sequence independent biological effects of phosphorothioate DNA (sulfated polyanion) and immunostimulation properties of CpG islands and did not result from true antisense mechanisms. Careful planning to avoid these pitfalls should make for strong platform for antisense with phosphorothioate backbone.

Phosphorothioates show increased binding to cellular proteins and components of the extracellular matrix as compared to natural phosphodiester oligonucleotides. This binding appears to be due to the polyanionic nature of these compounds; they behave similar to dextran sulfate and heparin sulfate. This binding can displace or mimic the binding of natural ligands to assorted proteins, such as receptors or adhesion molecules. In fact, any of the heparin-binding class of proteins may also bind phosphorothioates. Phosphodiester DNA is a polyanion and may nonspecifically bind proteins, but due to nuclease action has such a shortened lifespan that the impact of this effect is most likely limited.

In one more embodiment the present invention relates to an anti-sense oligonucleotide for use in treatment of viral diseases wherein the anti-sense oligonucleotide is selected from OT-101 or OT-101 in combination with any of SEQ ID NOS:5-12.

In one aspect the embodiment the present invention relates to the anti-sense oligonucleotide for use in the treatment of the viral disease induced by, but not limited to SARS, MERS, RSV, coronavirus, HIV, Ebola., Cytomegalovirus (CMV). Human herpes virus type 6 (HHV-6), Herpes simplex virus (HSV-1 and HSV2), Epstein-Barr virus (EBV), Hepatitis B virus (HBV).

In one more aspect of the embodiment the viral disease is COVID-19.

In one more embodiment of the present invention relates to a method of treating a fibrosis or any collagen related diseases, cancers, viral diseases, bacterial diseases and parasitic diseases, wherein the method comprises administering to the subject a therapeutically effective amount of anti-sense oligonucleotide sequence selected from OT-101, SEQ ID NO:5, SEQ ID NO:6, SEQ ID NO:7, SEQ ID NO:8, SEQ ID NO:9, SEQ ID NO:10, SEQ ID NO:11, SEQ ID NO:12 or a combination thereof.

In one aspect of the embodiment the method is for treating viral disease induced by, but not limited to SARS, MERS, RSV, corona, Ebola, Cytomegalovirus (CMV). Human herpes virus type 6 (HHV-6), Herpes simplex virus (HSV-1 and HSV2), Epstein-Barr virus (EBV), Hepatitis B virus (HBV).

In one more aspect of the embodiment the method is for treating bacterial, viral, or other forms cytokine induced pneumonia.

In yet another aspect of the embodiment the viral disease is COVID-19.

In yet another aspect of the embodiment the administration includes intravenous, intrathecal, intramuscular, oral, and any other acceptable route of administration.

In yet another aspect of the embodiment OT-101 anti-sense oligonucleotide inhibits TGF-beta. In yet another aspect of the embodiment TGF-beta is TGF-beta1, or TGF-beta2 or TGF-beta3.

In yet another aspect of the embodiment the antisense oligonucleotide being any combinations of antisense against TGF-beta, viral 5′Terminal, viral Transcription Regulatory Site, and the viral Frame Shift site.

Another important embodiment of the present invention relates to a method of treating TGF-beta storm.

In yet another aspect of the embodiment the present invention relates to a method of treating TGF-beta storm, the method involving treatment of TGF-beta storm with TGF-beta inhibitor, antiviral agents, IL-6 inhibitors, or any combination thereof.

In yet another aspect of the embodiment the present invention relates to TGF-beta inhibitor including mAb, small molecules target the active domain of TGF-beta.

In yet another aspect of the embodiment the present invention relates to TGF-beta inhibitor including mAb, small molecules, antisense, RNA therapeutics targets the activation of TGF-beta or activating protein.

In yet another aspect of the embodiment the present invention relates to TGF-beta inhibitor including mAb, small molecules, antisense, RNA therapeutics targets the virus replication or the virus binding and uptake or virus protein synthesis or virus replication.

In yet another aspect of the embodiment the present invention relates to the method of use of anti-sense oligonucleotides wherein the method comprises inhibition of viral binding to target cells.

In yet another aspect of the embodiment the present invention relates to the method of treatment of symptoms associated with viral infection.

In yet another aspect of the embodiment the present invention relates to the method including treatment of symptoms associated with respiratory viral infection.

In yet another aspect of the embodiment the present invention relates to the method including treatment of symptoms associated with coronavirus viral infection.

In yet another aspect of the embodiment A method of use of anti-sense oligonucleotide wherein the method comprises suppression of TGF-beta induced proteins including IL-6, TGFBIp.

In yet another aspect of the embodiment the method comprises suppression of symptoms due to TGF-beta inducible proteins such as IL-6, TGFBIp.

In yet another aspect of the embodiment the present invention relates anti-sense oligonucleotide is OT-101.

In yet another embodiment the present invention relates to a method of use of OT-101 to treat cytokine storm.

In yet another embodiment the present invention relates to a method of use of OT-101 to treat multiorgan inflammatory syndrome.

In yet another embodiment the present invention relates to a method of use of OT-101 to treat Kawasaki syndrome.

In yet another embodiment the present invention relates to a method of use of OT-101 to treat IgA vasculitis.

Throughout the description and claims of this specification, the phrases “comprise” and “contain” and variations of them mean “including but not limited to”, and are not intended to exclude other moieties, additives, components, integers or steps. Thus, the singular encompasses the plural unless the context otherwise requires. Wherever there is an indefinite article used, the specification is to be understood as contemplating plurality as well as singularity, unless the context requires otherwise.

Embodiments are further defined in the following examples. The following examples are for the purpose of illustration of the invention and not intended in any way to limit the scope of the invention.

### EXAMPLES

### Example 1. Artemisinin Antiviral Activity Against SARS-CoV2

Cao et al has demonstrated that TGF-β protein levels and mRNA levels in 4T1 breast cancer cells decrease after ART treatment. Sub-confluent 4T1 cells were harvested, washed once in serum-free media, and resuspended in PBS at a concentration of 5×105 cells/0.1 mL PBS. 0.1 mL of the cell suspension was then implanted into the abdominal mammary fat pad of female BALB/c mice subcutaneously. Once the tumor was palpable (5-7 days after implantation), the mice were randomized into either the control group (n=7; intraperitoneal injection with 200 μl sterile PBS daily for 20 days) or the ART group (n=10; intraperitoneal injection with 100 mg/kg ART dissolved in 0.2% DMSO daily for 20 days). TGF-β mRNA levels within the tumor significantly decreased after ART treatment (P<0.01).

Artemisinin could reduce early renal oxidative stress damage in diabetic nephropathy (DN) rats by inhibiting TGF-β1 protein expression in kidney tissues as well as activating the Nrf2 signaling pathway and enhancing the expression of antioxidant proteins, thereby exerting the protective effects on DN kidney. The western blot analysis showed that the expression of TGF-β1 in the kidney tissues of DN model rats (p<0.05) was significantly increased when compared with the normal control group. Artemisinin (25, 50, 75 mg/kg) restored near normal expression of TGF-β1 suppressing the expression of TGF-β1. Similarly, in lupus nephritis mice there was an increase in TGF-β expression. This overexpression was also ameloriated with Artemisinin treatment. Both RNA and protein levels were significantly reduced in comparison to the untreated control mice.

Suppression of TGF-β expression by OT-101 (an antisense against TGF-β) suppressed SARS-CoV and SARS-CoV-2 replication in the viral replication assays on Vero 76 cells was demonstrated in collaboration with Dr. Brett Hurst at Utah State University, part of the NIAID Antiviral Testing Consortium.

In this assay, compounds were serially diluted using eight half-log dilutions in test medium (MEM supplemented with 2% FBS and 50 μg/mL gentamicin) so that the starting (high) test concentration was 1000 μg/mL. Each dilution was added to 5 wells of a 96-well plate with 80-100% confluent Vero 76 cells.

Three wells of each dilution were infected with virus, and two wells remained uninfected as toxicity controls. Six wells were infected and untreated as virus controls, and six wells were uninfected and untreated as cell controls. SARS-CoV-2 virus suspensions were prepared to achieve the lowest possible multiplicity of infection (MOI) that would yield >80% cytopathic effect (CPE) within 5 days. M128533 was tested in parallel as a positive control.

On day 5 post-infection, once untreated virus control wells reached maximum CPE, plates were stained with neutral red dye for approximately 2 hours (15 minutes). Supernatant dye was removed and wells rinsed with PBS, and the incorporated dye was extracted in 50:50 Sorensen citrate buffer/ethanol for >30 minutes and the optical density was read on a spectrophotometer at 540 nm. Optical densities were converted to percent of cell controls and normalized to the virus control, then the concentration of test compound required to inhibit CPE by 50% (EC₅₀) was calculated by regression analysis. The concentration of compound that would cause 50% cell death in the absence of virus was similarly calculated (CC₅₀). The selective index (SI) is the CC₅₀ divided by EC₅₀.

Artemisinin, being a reported TGF-β inhibitor, also suppressed SARS-CoV-2 replication. OT-101 activity was superior to antisense specifically designed against SAR-CoV-2 genome along selected 5′-TERM, FS, and LTR. Nonspecific antisense (RSV) did not demonstrate any suppression. The data are shown in the table 1 below.

TABLE 1

| Compound | Virus | EC₅₀ | | CC₅₀ | SI |
|---|---|---|---|---|---|
| OT-101 | SARS-CoV | 7.6 | (1.24 uM) | >1000 | >130 |
| | (Urbani strain) | | | | |
| OT-101 | SARS-CoV | 26 | (4.23 uM) | >1000 | >38 |
| | (Urbani strain) | | | | |
| OT-101 | SARS-COV-2 | 2.0 | (0.33 uM) | >1000 | >500 |
| | USA_WA1/2020 | | | | |
| RSV | SARS-COV-2 | 620.0 | | >1000 | >1.6 |
| | USA_WA1/2020 | | | | |
| Artemisinin | SARS-COV-2 | 0.45 | (1.59 uM) | 61.0 | 140 |
| | USA_WA1/2020 | | | | |

| | | | | |
|---|---|---|---|---|
| M128533 | SARS-COV-2 | 0.012 | >10 | >830 |
| | USA_WA1/2020 | | | |
| Remdesivir | SARS-COV-2 | (0.77 uM) | (>100 uM) | >129.87 |

OT-101: TGF-β antisense; RSV: Negative control antisense; M128533: positive control; EC₅₀: 50% effective antiviral concentration (in μg/ml); CC₅₀: 50% cytotoxic concentration of compound without virus added (in μg/ml); SI=CC₅₀/EC₅₀; Source of SARS-CoV: Centers for Disease Control Stock 809940 (200300592). Source of SARS-CoV-2: World Reference Center for Emerging Viruses and Arboviruses (WRCEVA) at UTMB.

The anti-SARS-CoV-2 activity of Artemisinin was confirmed subsequently by two other laboratories:
1) By RTPCR method. For the antiviral assay, 4.8×106 Vero E6 cells were seeded onto 48-well cell-culture Petri dishes and grown overnight. After pretreatment with a gradient of diluted experimental compounds for 1 h at 37° C., cells were infected with virus at an MOI of 0.01 for 1 h. After incubation, the inoculum was removed, cells were washed with PBS, and culture vessels were replenished with fresh drug containing medium. At 24 h post-infection, total RNA was extracted from the supernatant and qRT-PCR was performed to quantify the virus yield.
2) by immunostaining for spike protein. VeroE6 cells seeded the previous day in 96-well plates were infected with SARS-CoV-2 and treated with the specified concentrations of test articles. After a 2-day incubation, infected cells were visualized by immunostaining for SARS-CoV-2 spike glycoprotein and counted automatically.

Furthermore, docking studies were performed to show that it is active against the initial binding and uptake of the virus. Artemisinin produced better Vina docking score than hydroxychloroquine (−7.1 kcal mol-1 for the best scoring artemisinin derivative vs. −5.5 kcal mol-1 for hydroxychloroquine). Artesunate, artemisinin and artenimol, showed two mode of interactions with Lys353 and Lys31 binding hotspots of the Spike protein.

Independently, when the ZINC natural library with a total of ˜203,458 Natural ligands was tested through blind docking against the S protein: human ACE2 complex, artemisinin was one of the top 4 candidates (Andrographolide, Artemisinin, Pterostilbene, and Resveratrol).

Artemisinin showed binding between the interface of S protein: human ACE2 complex. It is characterized: 1) 1 H-bond with Tyr-505 residue of the ACE2 receptor, 2) His-34 and Ala-387 formed alkyl and pi-alkyl contacts with the receptor and 3) Pro-389 forms a carbon H-bond.

### Example 2. Artemisinin-ARTI-19 Trial

Given our observation that artemisinin is potent antiviral against SARS-CoV-2 (COVID-19) better than remdesivir and chloroquine, and that artemisinin is commonly used herbal remedy worldwide, we set out to evaluate artemisinin in COVID-19 patients, to determine whether it is an effective treatment option for these patients. The ARTI-19 trial was cleared by Indian regulatory authorities, and is registered under the Clinical Trials Registry India (CTRI) with three active sites and additional sites to be added as the trial progresses and expands. ARTI-19 trial registration information can be found at: CTRI/2020/09/028044. Phase IV study to evaluate the safety and efficacy of Artemisinin on COVID-19 subjects as Interventional. http://ctri.nic.in/Clinicaltrials/advsearch.php.Site specific information is: 1) Government Medical College & Government General Hospital, Srikakulam, ANDHRA PRADESH. 2) Rajarshi Chhatrapati Shahu Maharaj Government Medical College and Chhatrapati Pramila Raje Hospital, MAHARASHTRA. And 3) Seven Star Hospital, MAHARASHTRA.

Aim of the study: A clinical study to observe the effect of Artemisinin in COVID-19 patients. These patients will have confirmed SARS-CoV-2 infection by RT-PCR and mild and moderate (hospitalized, without oxygen therapy) symptoms of COVID-19. These are patients with scores of 2-4 on the WHO Clinical Progression Scale.

Objective of the study: To evaluate the clinical effect of Artemisinin in COVID-19 patients (see above description of patients).

Primary endpoints of the study: Primary endpoints: Days to recovery in the signs and symptoms in COVID-19 patients by adding Artemisinin to SOC (see above description of patients) based on the WHO Clinical Progression Scale and (2) Assessment Criteria of Symptoms.

Description of the population to be studied: This pilot study will be carried out in 120 adult COVID-19 patients in Bangalore. These patients will have confirmed SARS-CoV-2 infection by RT-PCR and mild and moderate (hospitalized, without oxygen therapy) symptoms of COVID-19 and scores of 2-4 on the WHO Clinical Progression Scale. These patients are diagnosed at the hospital and fulfilling the criteria of diagnosis and inclusion criteria, willing to give their consent to participate in the clinical trial will be registered. These registered patients will be then randomly divided by computer generated randomization sequence in Group 1 or Group 2. Each registered patient will be provided the details of clinical trial by patient information sheet and after taking their consent, detailed history will be taken as per clinical research Perform a and generated data will be used for research. If the pilot study shows that Artemisinin improves on symptoms of COVID-19 patients when administered with SOC, an Expanded Clinical study will be carried out in 1,080 adult COVID-19 patients with the same study design as the Pilot Study.
Group 1—Treatment group: Artemisinin+SOC (Physician's choice).
Group 2—Control group: SOC (physician's choice).

Diagnostic criteria: These patients will have confirmed SARS-CoV-2 infection by RT-PCR and mild and moderate (hospitalized, without oxygen therapy) symptoms of COVID-19. These are patients with scores of 2-4 on the WHO Clinical Progression Scale.

### Inclusion Criteria:

1. Confirmed case of COVID-19 infection by laboratory tests for COVID-19.
2. Age limit: 21 to 60 years of age male, or non-pregnant or non-lactating female.
3. Patients with oxygen saturation higher than 95% and without any requirement of oxygen therapy or assisted ventilation.
4. Patients willing to give their informed consent to participate in the clinical trial.

### Exclusion Criteria:

1. COVID-19 positive patients above 60 years of age or below 21 years.
2. Patients on Immuno-suppression therapy.
3. Patients with associated renal or hepatic impairment.
4. Pregnant Women or lactating mothers.
5. Patients in advanced stage of disease requiring emergency medical intervention like pneumonia, bronchial asthma, organ failure.
6. Patients whom ventilator support is required.
7. Patients not willing to give their informed consent to participate in the clinical trial.
8. Patients with the following co-morbidities: insulin-dependent diabetes, hypertension with cardiac symptoms, morbid obesity with diabetes and/or hypertension. Uncontrolled diabetes. Uncontrolled hypertension.
Posology: In this, along with ongoing allopathic medicines will be given to patients who are confirmed for SARS-CoV-2 infection by RT-PCR and have mild or moderate (hospitalized, without oxygen therapy) symptoms of COVID-19. These are patients with scores of 2-4 on the WHO Clinical Progression Scale.
Results: The India arm of ARTI-19 global study is on track to complete enrollment of the first 120 cohort by Jan. 15th, 2021, of which 78 patients have already been randomized. Interim analysis of the first 78 pts (8 WHO scale 2 and 70 WHO scale 4 on randomization) was performed. WHO 10 point scale was used in this study.
   1) Of significant is 75% of WHO scale 4 patients exhibited a drop to WHO scale 3 by Day 2 of treatment with ARTIVeda™. WHO scale 3 does not require hospitalization.
   2) 40% of WHO scale 4 patients exhibited a drop to WHO scale 1 on day 5 of treatment with ARTIVeda™. Note: WHO scale 1 is asymptomatic.
   3) SOC=Standard of care including remdesivir/dex/heparin.
      Site specific analyses:
The SOC varied from site to site. Despite these patients being heavily treated with a range of antiviral agents as well as agents meant for COVID-19 symptoms such as Paracetamol for fever, these patients all improved faster when treated with ARTIVeda™+SOC.

### Recovery Analysis:

The median time to asymptomatic WHO scale of 1 was 5 days for ARTIVeda™ plus SOC as compared to 14 days for SOC alone. The differences were statistically significant meaning unlikely to happen by chance. The trend was more pronounced with higher initial disease status. Log rank statistics: WHO-scale 2,3,4: p=0.0369/RR=1.476 (0.8957-2.433), WHO-scale 3,4: p=0.026/RR=1.581 (0.9094-2.747), WHO-scale 4: p=0.0043/RR=2.038 (0.9961-4.168). RR=rate ratio for recovery.

Pharmaceutical Vegetable Capsule Compositions Comprising Artemisinin

### Example 3: Excipient Compatibility Study

The chemical compatibility of artemisinin with selected excipients is investigated. Excipients evaluated are: (1)Diluent (microcrystalline cellulose PH112, USP); (2) Stabilizer (Polysorbate 80 Dry Powder, IH); (3) Disintegrants (crospovidone USP, and croscarmellose sodium USP); (4) Antisticking agent (magnesium stearate, USP). Artemisinin is mixed in a 1:1 weight ratio with each excipient and the mixture is evaluated immediately after mixing, as well as after one month of accelerated aging at 40° C. and 75% relative humidity. Comparisons are made to artemisinin under the same conditions without excipient. It is found that there are no chemical incompatibilities with the selected excipients. All samples measurements of compatibility stability samples indicate artemisinin potency of 98.1% to 99.2% compared to control. There is no appreciable changes in the impurity profiles

TABLE 2
Excipients and artemisinin compatibility data

| | Artemisinin | Impurity | Impurity | Impurity |
|---|---|---|---|---|
| Excipient/Grade | (%) | RC1 (%) | RC2 (%) | RC3 (%) |
| No Excipient | 99.20 | 0.11 | 0.12 | ND* |
| (Control) | | | | |
| Microcrystalline | 99.00 | 0.10 | 0.13 | ND |
| Cellulose | | | | |
| Polysorbate 80 | 98.50 | 0.10 | 0.14 | ND |
| dry powder | | | | |
| Crospovidone | 98.10 | 0.08 | 0.10 | ND |
| Croscarmellose | 98.30 | 0.09 | 0.15 | ND |
| sodium | | | | |
| Magnesium | 98.20 | 0.09 | 0.11 | ND |
| Stearate | | | | |

*Not detectable;
Impurity Artemisitene (RC1);
Impurity 9-epiartemisinin (RC2);
Impurity Thujone (RC3)

### Example 4: Small Scale Testing of Capsule Formulations

Initial trials of capsule formulation development are performed for capsules comprising about 500 mg of artemisinin. Each formulation comprises a single diluent, a stabilizer, two disintegrants and an anticaking agent as described in Example 1. Formulations are prepared in 400 capsule sizes. The initial dry-blend process includes screening both the API (artemisinin) and each excipient through a 40-mesh screen, followed by manual bag blending. The API and all excipients, other than anticaking agent, are blended first, passed through 60-mesh screen followed by addition of anticaking agent and further blending. The resulting mixture is further screened through 40-mesh and then encapsulated in Size 0 vegetable capsule using a bench top filling machine using dosing discs and tamping pins to obtain consistent fill weights. Table 3 below shows the compositions tested in weight percentage.

TABLE 3
Trial Batch Composition Details

| S.No. | Ingredients | Specification | WND20244A | WND20254A | WND20255A | WND20263A |
|---|---|---|---|---|---|---|
| 1 | Artemisia | IH | 99.2% | 97.4% | 94.6% | 94.6% |
| | Annua Extract | | | | | |
| 2 | Microcrystalline | USP | 0.3% | 0.3% | 0.5% | 0.5% |
| | Cellulose PH112 | | | | | |
| 3 | Polysorbate 80 | USP | 0.0% | 1.9% | 0.0% | 0.0% |
| 4 | Polysorbate 80 | IH | 0.0% | 0.0% | 2.5% | 2.5% |
| | Dry Powder | | | | | |
| 5 | Cross povidone | USP | 0.0% | 0.0% | 1.0% | 1.0% |
| 6 | Cross | USP | 0.0% | 0.0% | 1.0% | 1.0% |
| | Carmellose | | | | | |
| 7 | Magnesium | USP | 0.5% | 0.5% | 0.5% | 0.5% |
| | stearate | | | | | |

Each of the compositions is tested for release of the artemisinin using a USP Type II apparatus, at 75 rpm in 900 mL of sodium phosphate buffer at pH 6.8 having 2% sodium lauryl sulfate. The percent of the artemisinin released from each composition is analyzed using HPLLC.

The First Trial (WND20244A) of the Artemisia annua Capsules was done as per formula given in Table-3. The capsule dissolution in sodium phosphate buffer of pH 6.8 with 2% (w/v) sodium lauryl sulfate at 75 rpm at 37° C. was observed very less i.e around 20%.

In this trial of WND20254A, with objective of improving the dissolution rate in sodium phosphate buffer of pH 6.8 with 2% (w/v) sodium lauryl sulfate at 75 rpm at 37° C., the surfactant liquid Polysorbate 80 was added which results in improving the dissolution rate up to 35% which is still far from the acceptable range. Also the texture of formulation was also found undesirable. The problem of dissolution was still not resolved in this trial so to achieve our target next trial batch was planned.

In the WND20255A trial dissolution rate problem was tried to be solved by replacing the liquid Polysorbate 80 with Polysorbate 80 Dry Powder and incorporating the disintegrant and superdisintegrant Crospovidone and Croscarmellose into the formula improved the dissolution rate and observed more than 70% at 45 minutes sodium phosphate buffer of pH 6.8 with 2% (w/v) sodium lauryl sulfate at 75 rpm at 37° C. during analysis .The final average fill weight of capsule was finalized with 500 mg. The problem of dissolution was resolved. Also found under the acceptance range. All physical parameters were complying as per target limit. Thus, all parameters were found satisfactory and send for analysis. Physical and chemical parameters also complies as per the requirement of an immediate release capsules for an oral dose.

### Example 5: Reproducibility Testing of Capsule Formulations

Artemisinin capsule dosage form was prepared by dry granulation process by using formula as given in Table 4.

TABLE 4
Trial Composition Details for Batches of Reproducibity Testing

| S. | | Functional | | | |
|---|---|---|---|---|---|
| No. | Ingredients | Category | WND20263A | WND20266A | WND20266A |
| 1 | Artemisia Annua | Active Ingredient | 94.6% | 94.6% | 94.6% |
| | Extract, IH | | | | |
| 2 | Microcrystalline | Diluent | 0.5% | 0.5% | 0.5% |
| | Cellulose PH112, | | | | |
| | USP | | | | |
| 3 | Polysorbate 80, | Stabilizer | 0.0% | 0.0% | 0.0% |
| | USP | | | | |
| 4 | Polysorbate 80 Dry | Stabilizer | 2.5% | 2.5% | 2.5% |
| | Powder, IH | | | | |
| 5 | Cross povidone, | Disintergrant | 1.0% | 1.0% | 1.0% |
| | USP | | | | |
| 6 | Crosscarmellose | Disintegrant | 1.0% | 1.0% | 1.0% |
| | sodium, USP | | | | |
| 7 | Magnesium | Anticaking agent | 0.5% | 0.5% | 0.5% |
| | stearate, USP | | | | |

Formulations are prepared in 1000 capsule sizes. The initial dry-blend process includes screening both the API (artemisinin) and each excipient through a 40-mesh screen, followed by Octagonal Blender (GR-17) blending. The API and all excipients, other than anticaking agent, are blended first, passed through 60-mesh screen followed by addition of anticaking agent and further blending. The resulting mixture is further screened through 40-mesh and then encapsulated in Size 0 vegetable capsule using a Semi Autometic Capsules Filling Machine (SA-9) to obtain consistent fill weights. Table 3 below shows the compositions tested in weight percentage.

To check reproducibility of the finalized formula and process, three reproducible batches of WND20263A, WND20266A & WND20268A were planned with same batch size, same formula and parameters, using similar equipment. Physical and chemical parameters were found satisfactory and reproducible in all aspects as shown in Table 4. These batches were also charged for stability.

Each batch is tested in a dissolution study of Type II, paddle using 900 sodium phosphate buffer of pH 6.8 with 2% (w/v) sodium lauryl sulfate at 75 rpm at 37° C. The results are shown below in Table 5. Results are similar across batches for artemisinin assay and dissolution values. The results for the batches are acceptable for an immediate release oral capsule, and this batch formula is therefore chosen for preparation of the large scale capsule preparation (GMP).

TABLE 5
Characteristics of capsules from Batches of the Reproducibility
Testing with the Final Lab Batch Formula

| S. | | | | | | | | |
|---|---|---|---|---|---|---|---|---|
| No. | Parameters | Limits | WND20263A | | WND20266A | | WND20268A | |
| 3 | Av. Fill weight of | 555.0 mg ± 5.0% | 549.87 | mg | 552.23 | mg | 554.68 | mg |
| | capsule | | | | | | | |
| 4 | Av. Weight | [555 mg blend part + | 652.13 | mg | 647.23 | mg | 649.68 | mg |
| | of capsule | 95.0 mg empty HPMC | | | | | | |
| | | Cap)] = 650.0 mg ± | | | | | | |
| | | 5.0% | | | | | | |
| 5 | Group weight of | 13.00 g ± 3.0% | 13.04 | g | 12.94 | g | 12.99 | g |
| | 20 capsules | | | | | | | |
| 6 | Dissolution | Not less than | Min: 85.62% | | Min: 86.43% | | Min: 85.08% | |
| | | 70.00% of labeled | Max: 89.55% | | Max: 89.98% | | Max: 90.05% | |
| | | amount in 45 minutes | Av: 87.58% | | Av: 88.20% | | Av: 87.56% | |
| 8 | Assay | | | | | | | |
| | Each | 450.00 mg to | 515.69 | mg | 518.84 | mg | 513.22 | mg |
| | HPMC | 550.00 mg | | | | | | |
| | capsule contains | | | | | | | |

| | | | | |
|---|---|---|---|---|
| Artemesia | 90.00% to | 103.14% | 103.77% | 102.64% |
| Annua(Artemisinin) | 110.00% | | | |
| | labelled amount | | | |

### Example 6: Scale Up of Vegetable Capsule Formulation (GMP)

Further studies are performed to prepare 11 kg batches of artemisinin vegetable capsules for GMP evaluation (current Good Manufacturing Practices as set by Food and Drug Administration). Based on the small-scale study results, the formula in Table 6is selected for further development.

TABLE 6
Batch formula for the Exhibit Batch (GMP) of 20,000 Capsules

| | | Rational for | % |
|---|---|---|---|
| Component | Grade | Use | (w/w) |
| Artemisia annua Extract | IH | API | 94.59 |
| Microcrystalline Cellulose | USP | Diluent | 0.45 |
| PH112 | | | |
| Polysorbate 80 Dry Powder | IH | Stabilizer | 2.45 |
| Cros povidone | USP | Disintegrant | 1.03 |
| Croscarmellose sodium | USP | Disintegrant | 1.03 |
| Magnesium Stearate | USP | Antisticking | 0.45 |
| | | agent | |

The processing steps in involved in the manufacturing of capsule dosage form is given below:

Sifting: Sift Artemisia annua Extract, and Microcrystalline cellulose PH112 through 40# sieve in double poly bag and load in Octagonal Blender & mix for 10 min,

Sifting of Lubricants: Sift Crospovidone, Cross Carmellose Sodium & Polysorbate 80 dry powder through 40# Sieve and Magnesium stearate through 60#.

Lubrication: Add sifted materials of step No: 2 (except Magnesium Stearate) to blend of Step no: 1 in Octagonal Blender and mix for 5.0 minutes.

Add sifted Magnesium stearate to blend in Octagonal Blender and mixed further for 3.0 minutes.

The blend is ready for analysis and further for filling in “0” size Transparent/C. Transparent # HPMC capsule shell. The theoretical average weight of veg capsule should be 96 mg±5.0%

Capsule Filling: Fill the dry mix blend into the Hopper of Semi—Automatic capsule filling machine. Set the capsule filling machine. After setting the machine, the in-process parameters are set and capsule filling in process control. First set the average fill weight

Average fill weight should be kept at 650.0 mg and theoretical Average weight of capsule should be 650.0 mg±3.0% [555 mg blend part+(95.0 mg empty HPMC Cap)] & all capsule filling parameters should be monitored and recorded.

Before taking capsule for polishing, dedust & Inspect the capsule for any denting, broken, spotted appearance.

The GMP analytical studies are performed, and it is found that the batch meets all GMP requirements. No adverse sticking of the blend to the tamping pins is observed.

### Artemisinin Dosing

### Example 7. Selection of 500 mg Daily for Five Days Oral Dose

12 healthy male Vietnamese subjects after a single, 500-mg, oral dose. The relatively small interindividual variation in pharmacokinetics does not seem to be of clinical significance. Tolerance to the single dose of artemisinin was good: no adverse effects were detected. Based on these results, a treatment schedule of 2×500 mg of artemisinin (oral dose) per day can be advised. This will result in adequate antimalarial plasma concentrations, despite poor bioavailability, and rapid elimination.

Eight healthy male, Vietnamese subjects were administered 1×250, 2×250 and 4×250 mg artemisinin capsules in a cross-over design with randomized sequence with a 7-day washout period between administrations. The pharmacokinetic results suggested artemisinin to be subject to high pre-systemic extraction. Artemisinin oral plasma clearance was about 400 L h-1 exhibiting a slight decrease with dose, although the effect was weak. There was a high correlation between artemisinin plasma concentrations determined at various time-points after drug administration and the AUCs after the 500 and 1000 mg doses, but less so after the 250 mg dose. Artemisinin was well tolerated with no apparent dose or time dependent effects on blood pressure, heart rate or body temperature.

A single-center, randomized, 4-sequence, open-label, crossover study conducted in 15 healthy male Vietnamese volunteers under fasting conditions with a washout period of 3 weeks between study visits was performed. A single oral dose of 160 or 500 mg of artemisinin was administered alone or in combination with piperaquine. Potential adverse events were monitored daily by the clinician and by using laboratory test results. Frequent blood samples were drawn for 12 hours after dose. Artemisinin was quantified in plasma using LC-MS/MS. Pharmacokinetic parameters were computed from the plasma concentration-time profiles using a noncompartmental analysis method.

This single-dose study found that the dose-normalized Cₘₐₓ, AUC₀₋ₗₐₛₜ, and AUC_{0-∞}. mean geometric differences between the test and reference formulations were relatively small (<40%) and will probably not have a clinical impact in the treatment of malaria infections.

The pharmacokinetics of artemisinin was studied in 11 Vietnamese patients with uncomplicated falciparum malaria after a single 500 mg oral dose. Parasites disappeared rapidly, with a mean parasite clearance time of 36 hr. No relationship was found between pharmacokinetics and the parasite elimination rate. Tolerance to the single dose of artemisinin was good. No adverse effects were detected. In conclusion, pharmacokinetics of a single dose of artemisinin for uncomplicated falciparum malaria is not different from findings in healthy subjects. A single dose of 500 mg of artemisinin is effective in reducing parasitemia in nonsevere falciparum malaria and is well-tolerated.

The immediate efficacies of two oral dosage regimens of artemisinin were investigated in 77 male and female adult Vietnamese falciparum malaria patients randomly assigned to treatment with either 500 mg of artemisinin daily for 5 days (group A; n=40) or artemisinin at a dose of 100 mg per day for 2 days, with the dose increased to 250 mg per day for 2 consecutive days and with a final dose of 500 mg on the fifth day (group B; n=37). Parasitemia was monitored every 4 h. The average parasite clearance time was longer in group B than in group A (means±standard deviations, 50±23 and 34±14 h, respectively; P<0.01). Artemisinin's pharmacokinetic parameters were similar on day 5 in both groups, although a significant increase in oral clearance from day 1 to day 5 was evident. Thus, artemisinin exhibited both dose- and time-dependent pharmacokinetics. The escalating dose studied did not result in higher artemisinin concentrations toward the end of the treatment period.

### Example 8. Establishment of 5 Day on/5 Day Off Cycle as Treatment Regimens

Artemisinin is mainly eliminated by hepatic transformation. To investigate whether the clearance of artemisinin in patients with liver cirrhosis is different from healthy volunteers, pharmacokinetic study was performed in male Vietnamese patients with Child B cirrhosis of the liver who received 500 mg of artemisinin orally. The results were compared to those found in a previous study in healthy subjects. The mean (±SD) area under the concentration time curve was 2365 (±1761) h ng/ml; the mean (±SD) clearance, 382 (±303)/L/h. The elimination half-life was 4 (±1.3) h extimated by log-linear regression and 2.4±0.9 h estimated by non-linear regression using a one-compartment first order elimination model. The mean (±SD) absorption time was 1.55 (±0.8) h. These results were not different from the results of healthy subjects and show that liver disease has no effect on the availability and clearance of oral artemisinin, indicating that artemisinin has an intermediate hepatic extraction ratio and that there is no significant first pass effect.

The influence of food intake on the pharmacokinetics of artemisinin was studied with six healthy Vietnamese male subjects. In a crossover study, artemisinin capsules (500 mg) were administered with and without food after an overnight fast. Plasma samples were obtained up to 24 h after intake of each drug. Measurement of artemisinin concentrations was performed by high-performance liquid chromatography with electrochemical detection. Tolerance was evaluated according to subjective and objective findings, including repeated physical examinations, routine blood investigations, and electrocardiograms. Pharmacokinetics were analyzed with a noncompartmental method and with a one-compartment model. This model had either zero-order or first order input. No statistically significant differences were found between the results of the two experimental conditions. Specifically, there were no consistent differences in parameters most likely to be affected by food intake, including absorption profile, absorption rate, bioavailability (f) (as reflected in area under the concentration time curve [AUC]), and drug clearance. Some mean±standard deviation parameters after food were as follows: maximum concentration of drug in serum (Cₘₐₓ), 443±224 μg×liter⁻¹; time to Cₘₐₓ, 1.78±1.2 h; AUC, 2,092±1,441 ng×ml⁻¹×h, apparent clearance/f, 321±167 liter×h⁻¹; mean residence time, 4.42±1.31 h; and time at which half of the terminal value was reached, 0.97±0.68 h. The total amount of artemisinin excreted in urine was less than 1% of the dose. We conclude that food intake has no major effect on artemisinin pharmacokinetics. In addition, we conclude tentatively that artemisinin is cleared by the liver, that this clearance does not depend on liver blood flow (i.e., that artemisinin is a so-called low-clearance drug), and that absorption of the drug is not affected by food intake.

Another important pharmacokinetic factor influenced by food is liver blood flow, and therefore bioavailability and/or systemic clearance. Because we found only trace amounts of unchanged artemisinin in urine, enzymatic, and thus most probably, hepatic, metabolism seems to the main route of elimination of artemisinin. Theoretically, biliary excretion is another possible route of elimination. The influence of changes in liver blood flow on pharmacokinetics depends on the relationship between liver blood flow and the intrinsic capacity of the liver to metabolize a drug (the so-called “intrinsic clearance”). When intrinsic clearance is high compared to liver blood flow, the rate-limiting factor in drug clearance is liver blood flow; changes in liver blood flow are thus expected to have an influence on pharmacokinetic parameters. When intrinsic clearance is low compared to liver blood flow, changes in liver blood flow do not affect clearance. Because we found no differences in the pharmacokinetics of artemisinin after food versus those before food, liver blood flow has no influence on the elimination or the bioavailability of artemisinin. Artemisinin is therefore probably a so-called low-clearance drug.

Fifteen subjects received four different dose regimens of a single dose of artemisinin as a conventional formulation (160 and 500 mg) and as a micronized test formulation (160 mg alone and in combination with piperaquine phosphate, 360 mg) with a washout period of 3 weeks between each period (i.e. four-way cross-over). Venous plasma samples were collected frequently up to 12 h after dose in each period. Artemisinin was quantified in plasma using liquid chromatography coupled with tandem mass spectrometry. A nonlinear mixed-effects modelling approach was utilized to evaluate the population pharmacokinetic properties of the drug and to investigate the clinical impact of different formulations.

The plasma concentration-time profiles for artemisinin were adequately described by a transit-absorption model with a one-compartment disposition, in all four sequences simultaneously. The mean oral clearance, volume of distribution and terminal elimination half-life was 417 L/h, 1210 L and 1.93 h, respectively. Influence of formulation, dose and possible interaction of piperaquine was evaluated as categorical covariates in full covariate approaches. No clinically significant differences between formulations were shown which was in accordance with the previous results using a non-compartmental bioequivalence approach.

The pharmacokinetics of the antimalarial artemisinin exhibited an unusual time dependency during a 7-day oral daily regimen of 500 mg in 10 healthy, male Vietnamese adults. Artemisinin areas under the plasma concentration-time curve (AUC) decreased to 34% (median) by day 4 with a further decrease by day 7 to only 24% of values obtained after the first day of administration. In seven subjects restudied after a 2-week washout period, artemisinin AUCs had almost normalized, demonstrating the reversibility of the time dependent drug disposition. The results suggest artemisinin exhibits an auto-inductive effect on drug metabolism of an unusual magnitude. This may partly explain why some patients on standard doses, due to sub-parasiticidal drug levels toward the end of a standard regimen, do not completely clear parasites.

Artemisinin induces its own metabolism even after a single dose, resulting in decreased concentrations after repeated administration. Increasing amounts of artemisinin in the liver compartment increased the rate of production of an enzyme precursor in a linear fashion, resulting in greater amounts of enzyme.

Twenty-four healthy males were randomized to receive either a daily single dose of 500 mg oral artemisinin for 5 days, or single oral doses of 100/100/250/250/500 mg on each of the first 5 days. Two subjects from each group were administered a new dose of 500 mg on one of the following days after the beginning of the study: 7, 10, 13, 16, 20, or 24. Artemisinin concentrations in saliva samples collected on days 1, 3, 5, and on the final day were determined by HPLC. Data were analysed using a semiphysiological model incorporating (a) autoinduction of a precursor to the metabolizing enzymes, and (b) a two-compartment pharmacokinetic model with a separate hepatic compartment to mimic the processes of autoinduction and high hepatic extraction.

Artemisinin was found to induce its own metabolism with a mean induction time of 1.9 h, whereas the enzyme elimination half-life was estimated to 37.9 h. The hepatic extraction ratio of artemisinin was estimated to be 0.93, increasing to about 0.99 after autoinduction of metabolism. The model indicated that autoinduction mainly affected bioavailability, but not systemic clearance. Non-linear increases in AUC with dose were explained by saturable hepatic elimination affecting the first-pass extraction.

Therefore, either scaling in dose or a break of 5 day following dosing for 5 days is necessary to prevent enzyme induction and to achieve high plasma concentration. Importantly, the dosing of artemisinin should not be affected by food intake or hepatic status.

### Example 9 Manufacturing: Physical, Chemical, and Pharmaceutical Properties, Formulation, and Route of Administration

Physical and Chemical Characterization

The product, ARTIVeda™, is a formulated Artemisinin derived from Artemisia annua.

Pharmaceutical Properties of the Investigational Medicinal Product

ARTIVeda™ is manufactured in a facility in compliance with current GMP and legal requirements. The final product is quality controlled by appropriate analytical methods (e.g. HPLC, pH, etc.) to confirm the identity and purity of ARTIVeda™. Analytical testing is performed according to common pharmaceutical standards (e.g. Pharm. Eur. and/or United States Pharmacopeia) for parenteral drugs.

ARTIVeda™ is supplied as a gelatin capsule for oral administration. The capusles are package in strips of 10 sufficient for two cycles of ARTIVeda™. The primary as well as secondary containers of the closure system fulfill international quality standards.

Administration: Preparation and Application Drug Product Manufacturing

ARTIVeda™ is administered as oral capsule as part of a 10 day treatment regimen of one capsule per day for 5 days follow by 5 days washout; and the cycle can be repeated. The drug product complies with The International Pharmacopoeia—Ninth Edition, 2019 Artemisinin (Artemisininum). The product is USP compliant as to USP 231, USP 232, and USP 233. The product is ICH and FDA compliant as to ICH Q3D and FDA Q3D(R1).

The manufacturing process is as shown in FIG. 6 and the batch formula for the commercial batches is shown in Table 1. The narrative summary of each unit operations of the manufacturing of the artemisinin capsule is described below:
1. Sifting: Sift Artemisia annua Extract, and Microcrystalline cellulose PH112 through 40# sieve in double poly bag and load in Octagonal Blender & mix for 10 min.
2. Sifting of Lubricants: Sift Crospovidone, Cross Carmellose Sodium & Polysorbate 80 dry powder through 40# Sieve and Magnesium stearate through 60#.
3. Lubrication: Add sifted materials of step No: 2 (except Magnesium Stearate) to blend of Step no: 1 in Octagonal Blender and mix for 5.0 minutes.
4. Add sifted Magnesium stearate to blend in Octagonal Blender and mixed further for 3.0 minutes
5. The blend is ready for analysis and further for filling in “0” size Transparent/C. Transparent #. HPMC capsule shell. The theoretical average weight of veg capsule should be 96 mg±5.0%.
6. CAPSULE FILLING: Fill the dry mix blend into the Hopper of Semi-Automatic capsule filling machine. Set the capsule filling machine. After setting the machine, the in-process parameters are set and capsule filling in process control. First set the average fill weight
7. Average fill weight should be kept at 650.0 mg and theoretical Average weight of capsule should be 650.0 mg±3.0% [555 mg blend part+(95.0 mg empty HPMC Cap)] & all capsule filling parameters should be monitored and recorded.
8. Before taking capsule for polishing, dedust & Inspect the capsule for any denting, broken, spotted appearance
9. POLISHING: Polish the capsule using polishing machine. Record the yield & store the capsule in double polyethylene bags
10. STORAGE OF FILLED CAPSULES: Store in controlled temperature of NMT 25° C. & NMT 32% respectively before sample will be released for packing.

TABLE 7
Batch Formula for the Commercial Batch

| | | Rational for | Qty/Cap, | Commercial Batch | | % |
|---|---|---|---|---|---|---|
| Component | Grade | Use | mg | 200000 Units | | (w/w) |
| Artemisinin | IH | API | 525.00 | 105.00 | kg | 94.59 |
| Microcrystalline | USP | Diluent | 2.500 | 0.500 | kg | 0.45 |
| Cellulose PH112 | | | | | | |
| Polysorbate 80 Dry | IH | Stabilizer | 13.600 | 2.720 | kg | 2.45 |
| Powder | | | | | | |
| Cros povidone | USP | Disintegrant | 5.700 | 1.140 | kg | 1.03 |
| Croscarmellose | USP | Disintegrant | 5.700 | 1.140 | kg | 1.03 |
| sodium | | | | | | |
| Magnesium Stearate | USP | Antisticking | 2.500 | 0.500 | kg | 0.45 |
| | | agent | | | | |
| Total Weight | | | 555.00 | 111.00 | kg | |

The following batches were manufactured with three exhibit batches matching the proposed commercial batch formula (Table 8). The batch descriptions are shown in Table 9 and testing results are shown in Table 10. The batches produces to date demonstrated robustness of the manufacturing process and stability of the product.

TABLE 8
Drug Product Batches

| S. No. | Batch No. | Mfg. Date | Batch Size | |
|---|---|---|---|---|
| 1. | WND20244A | 19 Sep. 2020 | 400 | Capsules |
| 2. | WND20254A | 28 Sep. 2020 | 400 | Capsules |
| 3. | WND20255A | 29 Sep. 2020 | 400 | Capsules |
| 4. | WND20263A | 5 Oct. 2020 | 1,000 | Capsules |
| 5. | WND20266A | 9 Oct. 2020 | 1,000 | Capsules |
| 6. | WND20268A | 9 Oct. 2020 | 1,000 | Capsules |

TABLE9
Drug Product Formula

| | Ingredients | Specs. | WND20244A | WND20254A | WND20255A | WND20263A | WND20266A | WND20268A |
|---|---|---|---|---|---|---|---|---|
| 1 | Artemisinin | IH | 525.00 | 525.00 | 525.00 | 525.00 | 525.00 | 525.00 |
| 2 | Microcrystalline | USP | 1.50 | 1.50 | 2.50 | 2.50 | 2.50 | 2.50 |
| | Cellulose PH112 | | | | | | | |
| 3 | Polysorbate 80 | USP | . . . | 10.00 | . . . | . . . | . . . | . . . |
| 4 | Polysorbate 80 | IH | . . . | . . . | 13.60 | 13.60 | 13.60 | 13.60 |
| | Dry Powder | | | | | | | |
| 5 | Cross povidone | USP | . . . | . . . | 5.70 | 5.70 | 5.70 | 5.70 |
| 6 | Cross | USP | . . . | . . . | 5.70 | 5.70 | 5.70 | 5.70 |
| | Carmellose | | | | | | | |
| | Sodium | | | | | | | |
| 7 | Magnesium | USP | 2.50 | 2.50 | 2.50 | 2.50 | 2.50 | 2.50 |
| | Stearate | | | | | | | |
| Total Weight | | | 529.00 | 539.00 | 555.00 | 555.00 | 555.00 | 555.00 |
| 8 | Transparent | IH | 95.00 mg | 95.00 mg | 95.00 mg | 95.00 mg | 95.00 mg | 95.00 mg |
| | # ‘0’ | | | | | | | |

TABLE 10
Drag Product Testing Results

| S. | | | | | | | | |
|---|---|---|---|---|---|---|---|---|
| No. | Parameters | Limits | WND20263A | | WND20266A | | WND20268A | |
| 1 | Description | HPMC Capsule size “0” | Complies | | Complies | | Complies | |
| | of filled | clear transparent/clear | | | | | | |
| | capsule | transparent containing | | | | | | |
| | | white crystalline white | | | | | | |
| | | powder | | | | | | |
| 2 | Identification | Should be positive for | Complies | | Complies | | Complies | |
| | | Artemisinin as per assay | | | | | | |
| | | method | | | | | | |
| 3 | Av. Fill | 555.0 mg ± 5.0% | 549.87 | mg | 552.23 | mg | 554.68 | mg |
| | weight of | | | | | | | |
| | capsule | | | | | | | |
| 4 | Av. Weight | [555 mg blend part + | 652.13 | mg | 647.23 | mg | 649.68 | mg |
| | of capsule | 95.0 mg empty HPMC | | | | | | |
| | | Cap)] = 650.0 mg ± 5.0% | | | | | | |
| 5 | Group | 13.00 g ± 3.0% | 13.04 | g | 12.94 | g | 12.99 | g |
| | weight of | | | | | | | |
| | 20 capsules | | | | | | | |
| 6 | Dissolution | Not less than 70.00% of | Min: 85.62% | | Min: 86.43% | | Min: 85.08% | |
| | | labeled amount in | Max: 89.55% | | Max: 89.98% | | Max: 90.05% | |
| | | 45 minutes | Av: 87.58% | | Av: 88.20% | | Av: 87.56% | |
| 7 | Related Substances | | | | | | | |
| | Impurity A | Not more than 0.5% | 0.04% | | 0.03% | | 0.04% | |
| | Impurity B | Not more than 0.5% | 0.11% | | 0.11% | | 0.11% | |
| | Any other | Not more than 0.2% | Not detected | | Not detected | | Not detected | |
| | secondary | | | | | | | |
| | impurity | | | | | | | |
| | Total | Not more than 2.0% | 0.15% | | | | 0.15% | |
| | Impurity | | | | | | | |
| 8 | Assay | | | | | | | |
| | Each | 450.00 mg to 550.00 mg | 515.69 | mg | 518.84 | mg | 513.22 | mg |
| | HPMC | | | | | | | |
| | capsule | | | | | | | |
| | contains | | | | | | | |
| | Artemisinin | 90.00% to 110.00% | 103.14% | | 103.77% | | 102.64% | |
| | | labelled amount | | | | | | |
| 9 | Microbial Limit Test | | | | | | | |
| | Total | Not more than 100,000 | 50 | cfu/g | 60 | cfu/g | 50 | cfu/g |
| | Microbial | cfu/g | | | | | | |
| | Plate Count | | | | | | | |
| | Total Yeast | Not more than 1,000 cfu/g | <10 | cfu/g | <10 | cfu/g | <10 | cfu/g |
| | & Mould | | | | | | | |

Pathogens

| | | | | |
|---|---|---|---|---|
| E coli | Should be absent/g | Absent/g | Absent/g | Absent/g |
| Salmonella | Should be absent/10 g | Absent/10 g | Absent/10 g | Absent/10 g |
| spp. | | | | |
| P. | Should be absent/g | Absent/g | Absent/g | Absent/g |
| aeruginosa | | | | |
| S. aureus | Should be absent/g | Absent/g | Absent/g | Absent/g |

### Example 10 Drug Stability: Handling and Storage Conditions Temperature Stability

Stability studies were performed according to International Conference on Harmonisation guidelines to obtain stability data of ARTIVeda™. According to these stability studies, ARTIVeda™ demonstrated at least 2 year shelf life when stored at RT [+25° C.±2° C./60% relative humidity (RH) for at least 24 months].

Stability plan includes accelerated stability (40° C./75% RH) at 0, 4, 8, and 12 weeks, and room temperature (25° C./60% RH) stability data at 0, 3, 6, 9, and 12 months for the drug product (Table 11). Current available stability data is summarized in Table 12.

TABLE 11
Stability Plan

| Strength | Container/Closure | Conditions | Sample Times | Batches |
|---|---|---|---|---|
| 500 mg | Alu-PVDC clear | 40° C. ± 2° C. | 0, 1, 3 and 6 | Batch# |
| | blister | 75% ± 5% RH | months | WND20263A |
| | | 30° C. ± 2° C. | 0.3, 6, 9, 12, 24 and | |
| | | 75% ± 5% RH | 36 months | |
| | | 25° C. ± 2° C. | 0, 3, 6, 9, 12 and 24 | |
| | | 60% ± 5% RH | months | |
| | | 5° C. ± 3° C. | 0, 3, 6, 9, 12 and 24 | |
| | | No humidity | months | |
| 500 mg | Alu-PVDC clear | 40° C. ± 2° C. | 0, 1, 3 and 6 | Batch# |
| | blister | 75% ± 5% RH | months | WND20266A |
| | | 30° C. ± 2° C. | 0, 3, 6, 9, 12, 24 and | |
| | | 75% + 5% RH | 36 months | |
| | | 25° C. ± 2° C. | 0, 3, 6, 9, 12 and 24 | |
| | | 60% ± 5% RH | months | |
| | | 5° C. ± 3° C. | 0, 3, 6, 9, 12 and 24 | |
| | | No humidity | months | |
| 500 mg | Alu-PVDC clear | 40° C. ± 2° C. | 0, 1, 3 and 6 | Batch# |
| | blister | 75% ± 5% RH | months | WND20268A |
| | | 30° C. ± 2° C. | 0, 3, 6, 9, 12, 24 and | |
| | | 75% ± 5% RH | 36 months | |
| | | 25° C. ± 2° C. | 0, 3, 6, 9, 12 and 24 | |
| | | 60% ± 5% RH | months | |
| | | 5° C. ± 3° C. | 0, 3, 6, 9, 12 and 24 | |
| | | No humidity | months | |

TABLE 12
Stability Data Summary

| | Accelerated | Room Temperature |
|---|---|---|
| | (40° C./75% RH), | (25° C./60% RH), |
| Specifications | 0, 4, 8, 12 weeks | 0, 3, 6, 9, 12 months |
| Assay (90-110%) | No Trend, All values vary | No Trend, All values vary |
| | between 98-102.1% | between 98.7-101.6% |
| Impurity RC1 (NMT 0.2%) | No Trend, All values | No Trend, All values |
| | (0.1-0.2%) | (0.1-0.2%) |
| Impurity RC2 (NMT 1.0%) | No Trend, All values | No Trend, All values |
| | (0.2-0.5%) | (0.2-0.5%) |
| Impurity RC3 (NMT 0.1%) | No Trend, Not detectable | No Trend, Not detectable |
| Any Unspecified Impurity | No Trend, All values are | No Trend, All values are |
| (NMT 0.1%) | (0.05-0.1%) | (0.05-0.1%) |
| Total Impurities (NMT | Upward Trend (0.7%), All | No trend (<0.3%), All values |
| 3.0%) | values are (0.5-0.7%) | are <0.3% |
| Dissolution | All Comply (70-80%) in 12 h | All Comply (70-80%) in 12 h |
| Moisture (NMT 5.0%) | No Trend, Values vary | No Trend, Values vary between |
| | between 3.1-4.2% | 2.1-2.5% |
| Description and Physical | All Comply | All Comply |
| Appearance | | |

Recommended Storage and Handling Conditions

To date, the recommended temperature condition for storage and transport of ARTIVeda™ is

+25° C.±2° C./60% RH.

### Example 11. Artemisinin Combination Products:

ArtemiC is a medical spray comprised of Artemisinin (6 mg/ml), Curcumin (20 mg/ml), Frankincense (=Boswellia) (15 mg/ml) and vitamin C (60 mg/ml) in micellar formulation for spray administration.

Patients were given up to 6 mg Artemisinin, 20 mg Curcumin, 15 mg Frankincense and 60 mg vitamin C given daily as an add-on therapy (in addition to standard care) in two divided doses, on Days 1 and 2.

Patients were randomized in a manner of 2:1 for study drug (ArtemiC) and Standard of Care to Placebo and Standard of Care.

Patients were followed-up last 2 weeks. During this time, patients were be monitored for adverse events.

Additional time is required for follow up (until hospital discharge) in order to check side effects and study drug efficacy.

Placebo, composed of the same solvent but without active ingredients, was given in the placebo group as add-on therapy, 2 times a day, on Days 1 and 2.

Study Purpose: This study is designed to evaluate the safety and efficacy of ArtemiC on patients diagnosed with COVID-19.

Methodology 50 adult patients who suffer from COVID-19 infection studied in parallel groups treated with active agent or placebo as add on to standard care.

Safety was assessed through collection and analysis of adverse events, blood and urine laboratory assessments and vital signs.

Phase II double-blind, placebo-controlled clinical trial for anti-inflammatory treatment ArtemiC, based on Swiss PharmaCan AG MyCell Enhanced™ delivery system technology, on those diagnosed with COVID-19, has met all the Phase II primary and secondary endpoints and demonstrated to improve the clinical recovery of the patients.

Key Trial Results

The comparison between the study and placebo groups before and after treatment is presented in table 13.

| Study visit | Study group | NEWS Score | P |
|---|---|---|---|
| Before treatment | ArtemiC ™ | 1.5152 | 0.54 |
| | Placebo | 1.8824 | |
| Day 15 | ArtemiC ™ | .5152 | 0.04 |
| | Placebo | 2.2353 | |

The Phase II trial involved 50 infected patients across three independent hospital sites in Israel and India, with 33 in the treatment group and 17 in the placebo group.

The full results have demonstrated to improve the health status of COVID-19 patients delivering a NEWS score of less than or equal to 2.

None of the patients in the treatment group required additional oxygen, mechanical ventilation or admission to intensive care where all of these events were reported in the placebo group.

The average NEWS score of patients in the placebo group was 2.25 statistically significantly higher (p<0.04) than in the treatment group −0.5.><0.04) than in the treatment group −0.5 NEWS score determines the degree of illness of a patient and prompts critical care intervention.

This was defined as a main tool for the estimation of COVID-19 patients clinical health status and improvement.

Different indications related to inflammation and cytokine storm, will be considered as future development goals and include a wide range of diseases related to cytokine storm such as autoimmune diseases, inflammatory GI diseases, flu and chemotherapy patients.

Primary Outcome Measures:
1. Time to clinical improvement, defined as a national Early Warning Score 2 (NEWS2) of </=2 Maintained for 24 Hours in comparison to routine treatment [Time Frame: 24 hours] patient will be assessed using a scoring table for changes in clinical signs
2. Percentage of participants with definite or probable drug related adverse events [Time Frame: 14 days] Adverse events caused by the study drug will be assessed

Secondary Outcome Measures:
1. Time to negative COVID-19 PCR [Time Frame: 14 days ]
2. Proportion of participants with normalization of fever and oxygen saturation through day 14 since onset of symptoms [Time Frame: 14 days ]
3. COVID-19 related survival [Time Frame: 14 days ]
4. Incidence and duration of mechanical ventilation [Time Frame: 14 days ]
5. Incidence of Intensive Care Init (ICU) stay [Time Frame: 14 days ]
6. Duration of ICU stay [Time Frame: 14 days ]
7. Duration of time on supplemental oxygen [Time Frame: 14 days ]

Inclusion Criteria:
1. Confirmed SARS-CoV-2 infection.
2. Hospitalized COVID-19 patient in stable moderate condition (i.e., not requiring ICU admission).
3. Subjects must be under observation or admitted to a controlled facility or hospital (home quarantine is not sufficient).

Exclusion Criteria:
1. Tube feeding or parenteral nutrition.
2. Patients who are symptomatic and require oxygen (Ordinal Scale for Clinical Improvement score >3) at the time of screening.
3. Respiratory decompensation requiring mechanical ventilation.
4. Uncontrolled diabetes type 2.
5. Autoimmune disease.
6. Pregnant or lactating women.
7. Any condition which, in the opinion of the Principal Investigator, would prevent full participation in this trial or would interfere with the evaluation of the trial endpoints.

### Example 12: ASOs Synthesized in the Present Invention:

Anti-sense oligonucleotides

| Name | Sequence (5′-3′) | 2′MOE |
|---|---|---|
| 5TERM (1-20) | (SEQ ID NO: 59) | No |
| | G*G*T*A*G*G*T*A*A*A*A*A*C*C*T*A*A*T*A*T* | |
| TRS1 (53-72) | (SEQ ID NO: 60) | No |
| | G*T*TC*^{Me}G*T*T*T*A*G*A*G*A*A*C*^{Me}*G*A*T*C*^{Me} | |
| TRS2 (56-76) | (SEQ ID NO: 61) | No |
| | T*A*A*A*G*T*T*C*^{Me}*T*T*T*A*G*A*G*A*A*C*^{Me}*G* | |
| FS (13,458- | (SEQ ID NO: 62) | No |
| 13,472) | A*G*C*^{Me}c*^{Me}C*^{Me}T*G*T*A*G*A*C*^{Me}*A*C*^{Me} | |
| 5TERM (1-20) | (SEQ ID NO: 63) | Yes |
| | G*G*T*A*G*G*T*A*A*A*A*A*C*C*T*A*A*T*A*T* | |
| TRS2-2 53-72 | (SEQ ID NO: 64) | No |
| | C*G*T*T*T*A*G*A*G*A*A*C*A*G*A*T*C*T*A*C* | |
| TRS2-2 53-72 | (SEQ ID NO: 65) | Yes |
| | C*G*T*T*T*A*G*A*G*A*A*C*A*G*A*T*C*T*A*C* | |
| FS (13,458- | (SEQ ID NO: 66) | Yes |
| 13,472) | A*G*C*^{Me}c*^{Me}C*^{Me}T*G*T*A*T*A*C*^{Me}*A*C*^{Me} | |
| FS-2a (13539- | (SEQ ID NO: 67) | No |
| 13558) | C*A*T*T*G*T*A*G*A*T*G*T*C*A*A*A*A*G*C*C* | |
| RSV1 | (SEQ ID NO: 68) | No |
| | C*T*C*C*C*T*C*A*T*G*G*T*G*G*C*A*G*T*T*G*A* | |

1) *PS
2) Substitution at the 5-position of the cytosine (C) with a methyl group is indicated by ^{Me}

Analysis by OligoEvaluator (Sigma)

5TERM (1-20)
5′ G*G*T*A*G*G*T*A*A*A*A*A*C*C*T*A*A*T*A*T-3′ (SEQ ID NO: 69)

| Base | Molecular | Extinction | Oligo | μg/OD | Length | Tm | GC | GC | Run | Secondary | Primer | |
|---|---|---|---|---|---|---|---|---|---|---|---|---|
| Count | Weight | Coefficient | Type | at 260 nm | (bp) | (^{O} C.) | % | Clamp | Length (bp) | Structure | Dimer | BLAST |
| A = 9, | 6173.1 | 212.7 | No | 29.0 | 20 | 48.5 | 30.0 | 0 | 5 | Moderate | Yes | Sequence |
| U = 0, | | | Mod | | | | | | | | | |
| G = 4, | | | | | | | | | | | | |
| C = 2, | | | | | | | | | | | | |
| T = 5, | | | | | | | | | | | | |
| I = 0, | | | | | | | | | | | | |
| Total = 20 | | | | | | | | | | | | |

TRS1 (53-72)
5′-G*T*TC*G*T*T*T*A*G*A*G*A*A*C*A*G*A*T*C 3′ (SEQ ID NO: 70)

| Base | Molecular | Extinction | Oligo | μg/OD | Length | Tm | GC | GC | Run | Secondary | Primer | |
|---|---|---|---|---|---|---|---|---|---|---|---|---|
| Count | Weight | Coefficient | Type | at 260 nm | (bp) | (^{O} C.) | % | Clamp | Length (bp) | Structure | Dimer | BLAST |
| A = 6, | 6156.1 | 2020.0 | No | 30.5 | 20 | 52.8 | 40.0 | 1 | 3 | Moderate | Yes | Sequence |
| U = 0, | | | Mod | | | | | | | | | |
| G = 5, | | | | | | | | | | | | |
| C = 6, | | | | | | | | | | | | |
| T = 6, | | | | | | | | | | | | |
| I = 0, | | | | | | | | | | | | |
| Total = 20 | | | | | | | | | | | | |

TRS2 (56-76)
5′T*A*A*A*G*T*T*C*G*T*T*T*A*G*A*G*A*A*C*A*G 3′ (SEQ ID NO: 71)

| Base | Molecular | Extinction | Oligo | μg/OD | Length | Tm | GC | GC | Run | Secondary | Primer | |
|---|---|---|---|---|---|---|---|---|---|---|---|---|
| Count | Weight | Coefficient | Type | at 260 nm | (bp) | (^{O} C.) | % | Clamp | Length (bp) | Structure | Dimer | BLAST |
| A = 8, | 6493.3 | 218.9 | No | 29.7 | 21 | 53.1 | 33.3 | 1 | 3 | Moderate | No | Sequence |
| U = 0, | | | Mod | | | | | | | | | |
| G = 5, | | | | | | | | | | | | |
| C = 2, | | | | | | | | | | | | |
| T = 6, | | | | | | | | | | | | |
| I = 0, | | | | | | | | | | | | |
| Total = 21 | | | | | | | | | | | | |

FS (13,458-13,472)
5′ A*G*C*C*C*T*G*T*A*T*A*C*G*A*C 3′ (SEQ ID NO: 72)

| Base | Molecular | Extinction | Oligo | μg/OD | Length | Tm | GC | GC | Run | Secondary | Primer | |
|---|---|---|---|---|---|---|---|---|---|---|---|---|
| Count | Weight | Coefficient | Type | at 260 nm | (bp) | (^{O} C.) | % | Clamp | Length (bp) | Structure | Dimer | BLAST |
| A = 4, | 4761.9 | 144.8 | No | 32.9 | 15 | 47.4 | 53.3 | 2 | 3 | None | No | Sequence |
| U = 0, | | | Mod | | | | | | | | | |
| G = 3, | | | | | | | | | | | | |
| C = 5, | | | | | | | | | | | | |
| T = 3, | | | | | | | | | | | | |
| I = 0, | | | | | | | | | | | | |
| Total = 15 | | | | | | | | | | | | |

RSV1
5′-C*T*C*C*C*T*C*A*T*G*G*T*G*G*C*A*G*T*T*G*A-3′ (SEQ ID NO: 73)

| Base | Molecular | Extinction | Oligo | μg/OD | Length | Tm | GC | GC | Run | Secondary | Primer | |
|---|---|---|---|---|---|---|---|---|---|---|---|---|
| Count | Weight | Coefficient | Type | at 260 nm | (bp) | (^{O} C.) | % | Clamp | Length (bp) | Structure | Dimer | BLAST |
| A = 3, | 6734.4 | 193.0 | No | 34.9 | 21 | 69.7 | 57.1 | 1 | 3 | Very Weak | No | Sequence |
| U = 0, | | | Mod | | | | | | | | | |
| G = 6, | | | | | | | | | | | | |
| C = 6, | | | | | | | | | | | | |
| T = 6, | | | | | | | | | | | | |
| I = 0, | | | | | | | | | | | | |
| Total = 21 | | | | | | | | | | | | |

Results: SARS antiviral assay result for OT-101
1. Prepare 96-well plates of the desired cell line and incubate overnight. Seed platesat a cell concentration that will yield 80-100% confluent monolayers in each well after overnight incubation.
2. Prepare 8 half-log, serial dilutions in test medium with the highest test compound concentration of 1000 μg/mL.
4. Add 100 μL of each concentration to 5 test wells on the 96-well plate. Infect 3 wells of each dilution with the test virus in test medium (≤100 CCID₅₀ per well for most viruses). Add test medium with no virus to 2 wells (uninfected toxicity controls).
5. Infect 6 wells as untreated virus controls.
6. Add media only to 6 wells as cell controls.
7. Test a known active compound in parallel as a control.
8. Incubate at 37 C +5% CO2 until CPE is apparent.
9. After cytopathic effect (CPE) is observed microscopically, stain with 0.011% neutral red dye for approximately 2 hours. Siphon off neutral red dye (optionally rinse once with PBS to remove residual, unincorporated dye).
10. Perform CPE quantitation versus drug concentration to determine EC50
11. OT-101 had an 50% effective concentration of 7.6 μg/ml and was not toxic at the highest dose of 1000 μg/ml giving a Safety Index (SI) value of >130 which we would consider highly active.
12. Safety Index=Toxic dose/Efficacy dose. The wider the range the more safe the drug is.
13. As OT-101 has been through multiple clinical trials with more than 200 pts treated, there should be no problem putting OT-101 into clinical testing against COVID19
14. OT-101 is expected to have multiple mechanism of action against COVID-19: 1) Antiviral activity, 2) Anti-pneumonia activity and 3) Anti-viral binding to its receptor.

Test media:

MEM+2% FBS and 50 ug/mL gentamicin for most viruses

For flu: MEM+10 U/mL trypsin & 1 ug/mL EDTA for influenza Other special media if required depending on the virus and cell type

### Example 13. Testing for SARS-CoV2 with Antisense. Antisense Molecules Described in Example 1 and Also OT-101 (Antisense Against TGF-beta 2)

OT-101 (Trabedersen) and the ten antisense compounds against SARS-CoV-2 were solubilized in sterile saline to prepare 20 mg/mL stock solutions which were sterile filtered through a 0.2 μM low protein binding filter. Compounds were serially diluted using eight half-log dilutions in test medium (MEM supplemented with 2% FBS and 50 μg/mL gentamicin) so that the starting (high) test concentration was 1000 μg/mL. Each dilution was added to 5 wells of a 96-well plate with 80-100% confluent Vero 76 cells.

Three wells of each dilution were infected with virus, and two wells remained uninfected as toxicity controls. Six wells were infected and untreated as virus controls, and six wells were uninfected and untreated as cell controls. SARS-CoV-2 virus suspensions were prepared to achieve the lowest possible multiplicity of infection (MOI) that would yield >80% cytopathic effect (CPE) within 5 days. M128533 was tested in parallel as a positive control.

On day 5 post-infection, once untreated virus control wells reached maximum CPE, plates were stained with neutral red dye for approximately 2 hours (15 minutes). Supernatant dye was removed and wells rinsed with PBS, and the incorporated dye was extracted in 50:50 Sorensen citrate buffer/ethanol for >30 minutes and the optical density was read on a spectrophotometer at 540 nm. Optical densities were converted to percent of cell controls and normalized to the virus control, then the concentration of test compound required to inhibit CPE by 50% (EC₅₀) was calculated by regression analysis. The concentration of compound that would cause 50% cell death in the absence of virus was similarly calculated (CC₅₀). The selective index (SI) is the CC₅₀ divided by EC₅₀.

Antiviral activity against SARS-CoV-2 for each compound is shown in below. Cytotoxicity was observed for TRS1 (53-72), FS (13,458-13,472), and STERM (1-20) MOE. High antiviral activity was observed with the following compounds: OT-101, 5TERM (1-20), TRS1 (53-72), FS (13,458-13,472), 5TERM (1-20) MOE, TRS2-2 53-72, FS-2a (13539-13558). The positive control compound performed as expected.

TABLE 14

| | EC₅₀ | CC₅₀ | SI |
|---|---|---|---|
| OT-101 | 2.0 | >1000 | >500 |
| 5TERM (1-20) | 7.1 | >1000 | >140 |
| TRS1 (53-72) | 7.6 | 720 | 95 |
| TRS2 (56-76) | 73 | >1000 | >14 |
| FS (13,458-13,472) | 5.2 | 430 | 53 |
| 5TERM (1-20) MOE | 4.9 | 610 | 120 |
| TRS2-2 53-72 | 1.9 | >1000 | >530 |
| TRS2-2 53-72 MOE | 62 | >1000 | >16 |
| FS (13.458-13.274)MOE | 25 | >1000 | >40 |
| FS-2a (13539-13558) | 17 | >1000 | >59 |
| RSV | 620 | >1000 | >1.6 |
| Remdesivir | (0.77 uM) | (>200 uM) | >130 |
| M128533 (positive | 0.012 | >10 | >830 |
| control) | | | |

RSV-Negative control antisense/M128533-positive control. EC₅₀: 50% effective antiviral concentration (in μg/ml)/CC₅₀: 50% cytotoxic concentration of compound without virus added (in μg/ml)/SI=CC₅₀/EC₅₀. Source of SARS-COV-2: the World Reference Center for Emerging Viruses and Arboviruses (WRCEVA) at UTMB. Units are in μg/mL for test compounds and M128533.

### Example 14. TGF-Beta Inhibition Activity of OT-101.

The effect of trabedersen on TGF-β2 secretion was analyzed in cell lines of human HGG, human pancreatic carcinoma, malignant melanoma, colorectal carcinoma, and other tumors (prostate carcinoma, renal cell carcinoma, and non-small cell lung carcinoma). The TGF-β2 concentration in cell culture supernatants after treatment with trabedersen for 7 days was analyzed. Trabedersen reduced TGF-β2 secretion in the human HGG cell line A-172 compared to untreated controls at all concentrations tested; the highest inhibitory effect of 64% was observed with 10 trabedersen (Also known as OT-101). Trabedersen displayed very potent activity also in other cell lines, and concentration-dependently reduced TGF-β2 secretion compared to untreated control. The trabedersen concentration required to achieve half maximal inhibition of TGF-β2 secretion (half maximal inhibitory concentration [IC₅₀ ]) in vitro (without carrier) was determined for human HGG, pancreatic carcinoma, malignant melanoma, and colorectal carcinoma cells to be in the range of 2 to 5 Furthermore, down-regulation of TGF-β2 was also demonstrated in human malignant melanoma cells, i.e. MER 116 and RPMI 7951 (OT-101) and human cell lines originating from other tumor types such as non-small cell lung carcinoma, prostate carcinoma, renal clear cell carcinoma.

Results from in vitro experiments clearly demonstrated that trabedersen has high potency to inhibit TGF-β2 secretion.

Reversal of TGF-β-Induced Immunosuppression

TGF-β2 inhibits proliferation of lymphocytes and suppresses lymphocyte-mediated cytotoxicity directed against tumor cells. Targeted inhibition of TGF-β2 by trabedersen should re-establish cytotoxic activity of immune cells against human tumors.

Human HGG cells were obtained from surgical specimens of 5 patients. PBMCs from these patients were activated with human recombinant IL-2 to generate lymphokine-activated killer (LAK) cells as effector cells, which are known to lyse most autologous and allogenic fresh human tumor cells. LAK cell-mediated cytotoxicity against the patient derived autologous HGG cells (target cells) was tested in a cell co-culture system using the calcein-release assay. Trabedersen clearly enhanced autologous cytotoxicity against human HGG cells with a mean of 40% (untreated control 16%). The increase in antitumor activity ranged from 41% to 520% compared to untreated control.

The effects of trabedersen were evaluated in an allogenic cellular cytotoxicity test system using human pancreatic cancer cell lines as target cells and PBMCs from healthy donors as effector cells. Effector cells were incubated in cell supernatants of trabedersen-treated tumor cells before coincubation with the target cells in the cell-mediated cytotoxicity assay. Human immune cells cultivated with supernatants of tumor cells (PA-TU-8902) treated with trabedersen/Lipofectin showed an increased antitumor activity in comparison to untreated control and Lipofectin control at different ratios of human immune effector and pancreatic cancer target cells. These results were confirmed with PBMCs from a different healthy donor as well as another human pancreatic carcinoma cell line (Hup-T3).

Trabedersen reversed the suppression of immune cells by human tumor cells via inhibition of TGF-β2 secretion. These results showed that the antitumor activity of the human immune cells was clearly enhanced after treatment with trabedersen and underscored the potential therapeutic benefit of this approach.

Efficacy in TGF-β Expressing Xenograft Models

Studies in functional in vivo test systems demonstrated that: (i) OT-101 has minor antitumor activities on its own. However it was able to synergize and increase the activity of Paclitaxel and Dacarbazine. OT-101 was unable to synergize with Gemcitabine. Significant antitumor activity was achieved at human dose equivalent to 80 mg/m²/day which is well below the optimized clinical dose used for IV infusion of patients at 140 mg/m²/day.

Evaluation of 0T-101-Induced Anti-Tumor Activity in Orthotopic C8161 Human Melanoma Model in Female BALB/c nu/nu Mice C8161. Sixty female athymic nude mice were intradermally inoculated with 0.5×106 C8161 human melanoma cells and randomized into six groups of 10 mice. Three groups received monotherapy treatment with either OT-101 (16 mg/kg) (Group 2) or DTIC (1 or 10 mg/kg; Groups 3, 4). Two groups received combination therapy with OT-101/DTIC at 16/1 mg/kg or 16/10 mg/kg (Groups 5 and 6). Vehicle (0.9% saline, Groups 1, 3, and 4) and OT-101 were administered 3 times/week via subcutaneous injection (SC). Vehicle (0.9% saline, Groups 1 and 2) and DTIC (1 or 10 mg/kg) were administered via intraperitoneal injection (ip) four times/week beginning day 14. Mice were monitored for adverse effects, body weight and tumor size three times weekly. The tumor, lungs, liver and kidneys were excised from all mice at termination and weighed. Tumor growth was suppressed versus control group 1 by 2%, −2%, 78%, 27%, and 92% on day 42 for group 2, 3, 4, 5, and 6, respectively. Using Kruskal-Wallis test to compare all 6 groups, there is a significant difference in the tumor volume growth (P<0.0001). ANOVA statistic of repeated measurements versus control group 1 was performed and the P-values are non significant (ns), ns, <0.0001, ns, and <0.0001 for group 2, 3, 4, 5, and 6, respectively. DTIC inhibition of tumor growth was enhanced when OT-101 was combined with either the low dose DTIC (group 5 vs. 3) or high dose DTIC (group 6 vs. 4), with 29% and 14% improvement, respectively. Anti-tumor activity of the combination, group 6, was significantly better than high dose DTIC, group 4 (P=0.038).

In Vivo Evaluation of Taxol and Trabedersen (OT-101) Against Human Glioblastoma U87 MG Xenograft Model in Nude Mice. A subcutaneous U87 MG xenograft model was established in nude mice to test the efficacy of monotherapy of paclitaxel (Taxol) and Trabedersen (OT-101) and combination therapy of Taxol with Trabedersen in two dosing schedules against human glioblastoma. The optimal dosages of test agents were decided based on previous dose-finding studies at a given schedule and their clinical doses for a better prediction of clinical outcomes. Overall, the combination of Taxol with Trabedersen appeared tolerable and demonstrated enhanced anti-tumor efficacy in the U87 glioblastoma xenograft model. The combination of Taxol with Trabedersen was shown to have a significant synergistic relationship in vivo with a schedule of Trabedersen followed by Taxol resulting in enhanced antitumor activity as well as increased survival in mice.

In Vivo Evaluation of Taxol and Trabedersen (OT-101) Against Human Ovarian Adenocarcinoma in SK-OV-3 Xenograft Model in Nude Mice. A subcutaneous SK-OV-3 ovarian cancer xenograft model was established in nude mice to test the efficacy of monotherapy of paclitaxel (Taxol) and Trabedersen (OT-101) and combination therapy of Taxol with Trabedersen in two dosing schedules against human ovarian cancer. The optimal dosages of test agents were decided based on previous dose-finding studies at a given schedule and their clinical doses for a better prediction of clinical outcomes. Overall, the combination of 10 mg/kg Taxol with 32 mg/kg Trabedersen appeared tolerable and demonstrated enhanced anti-tumor efficacy in the SK-OV-3 ovarian cancer xenograft model. The combination of Taxol with Trabedersen was shown to have a significant synergistic relationship in vivo with a schedule of Trabedersen followed by Taxol (D7 administration) resulting in enhanced antitumor activity as well as increased survival in mice.

Biological Activity of 3′-Truncated (n-1)-(n-4) Trabedersen

The biological activity of truncated trabedersen, i.e. metabolites, in comparison to full-length trabedersen with respect to inhibition of TGF-β2 secretion was tested in the human HGG cell line A-172. Depending on the concentration, the biological activity of 3′-truncated (n-1) and (n-2) trabedersen was in the same range as full-length trabedersen at 5 or 10 μM, whereas the biological activity of the shorter fragments (3′-truncated (n-3) and (n-4)) was lower.

Effect on Viability and Proliferation of Human Peripheral Blood Mononuclear Cells

Cell viability was assessed following treatment of human PBMCs with trabedersen in vitro using the Trypan blue exclusion test. Freshly isolated, IL-2 activated PBMCs from healthy donors and HGG patients were incubated either without trabedersen or with 1, 5, 10 or 80 μM trabedersen for 2, 3, 6, 7, 14, and 21 day. No relevant effects on the viability of PBMCs either from healthy donors or HGG patients were observed up to 21 day. There was no difference in cell viability between untreated and trabedersen treated cells. Proliferation of PBMCs treated with 5, 10 or 50μM trabedersen, respectively, was within the range of variation. PBMC proliferation after 7 days of treatment with 80 μM trabedersen was slightly reduced (67% of untreated control cells).

While tumor cell proliferation was inhibited by trabedersen, viability and proliferation of PBMCs was not significantly negatively affected at clinically applied concentrations.

### Example 14. Clinical Efficacy of OT-101 Against TGF-Beta Expressing Solid Tumors

The clinical development program currently comprises 1 Phase I/II study of i.v. administered trabedersen in patients with solid tumors and 3 Phase I/II studies, 1 randomized and active-controlled Phase IIb study, and 1 Phase III study of locally administerd (intratumoral) trabedersen in patients with recurrent or refractory high-grade glioma.

Intravenous Administration in Patients with Solid Tumors

A Phase I/II Study P001 was conducted to investigate the i.v. administration of trabedersen in patients with solid tumors (i.e. advanced pancreatic carcinoma, malignant melanoma, or colorectal carcinoma).

Study Description

P001 is a completed Phase I/II dose escalation study. Primary objective is the determination of the MTD as well as the DLT of 2 cycles as core treatment and up to 8 optional extension cycles of trabedersen administered i.v. for 4 or 7 d every other week, as described in the following. The study followed a classical cohort design with 3 evaluable patients per cohort. Patients treated with the 1^{st} schedule received trabedersen continuously for 7 d, followed by a treatment-free interval of 7 d for each treatment cycle (7-d-on, 7-d-off). After the MTD had been reached for this schedule, a 2^{nd} schedule of 4 d trabedersen administration, followed by a treatment-free interval of 10 d for each treatment cycle was started (4-d-on, 10-d-off). In this treatment schedule the MTD has not been reached.

Objectives and Treatment

Primary objective of the study is the determination of the maximum tolerated dose (MTD) as well as the dose limiting toxicity (DLT) of two cycles of Trabedersen administered every other week. Secondary objectives include safety and tolerability, pharmacokinetic profile and potential antitumor activity of intravenous Trabedersen treatment.

Trabedersen was administered as i.v. continuous infusion for 4 or 7 days every other week via an implanted subcutaneous port system connected to a portable pump and with a flow rate of 0.8 mL/h. The core treatment consisted of 2 treatment cycles. Up to 8 optional extension cycles were administered in case of clinical benefit.

Main Inclusion and Exclusion Criteria

The study population included adult patients (18-75 years) with a histologically or cytologically confirmed diagnosis of either
pancreatic cancer Stage III or IV (American Joint Committee on Cancer, AJCC 2002; corresponds to AJCC 1997 Stage IVA or IVB),
malignant melanoma Stage III or IV (AJCC 2002), or
colorectal cancer Stage III or IV (AJCC 2002).

Other important inclusion criteria were a Karnofsky performance status of ≥80%, adequate organ function and recovery from acute toxicity caused by any previous therapy. Patients were either not or no longer amenable to established forms of therapy.

Main exclusion criteria included a history of brain metastasis and radiation therapy within 12 weeks, tumor surgery within 4 weeks or any other therapy with established antitumor effects within 2 weeks before study entry.

Dose Escalation

The dose escalation followed a classical cohort design with at least 3 and up to 6 patients per cohort receiving Trabedersen. The starting dose was chosen based on the Lowest-Observed-Adverse-Effect Level (LOAEL) determined in monkeys as the most relevant species. LOAEL was found to be equivalent to 48 mg/m²/day in human adults and therefore, the starting dose was set at 40 mg/m²/day (equivalent to approx. 1 mg/kg b.w./day). The Data and Safety Monitoring Board (DSMB) regularly reviewed available safety and efficacy data before each escalation step. Generally, if patients of one cohort had tolerated the therapy, the next cohort received the next higher dose. Toxicity was assessed based on National Cancer Institute-Common Toxicity Criteria (NCI-CTC, version 2). A DLT was defined as an at least possibly related, medically important adverse event, of NCI-CTC grade 3 or 4, a worsening by ≥2 grades from baseline for renal or hepatic toxicities, a worsening by ≥3 grades from baseline for other laboratory parameters, or other toxicities considered dose-limiting by the investigator. If more than 2 patients of a cohort had DLTs, the next lower dose was defined as MTD. Dose-escalation had to be stopped if MTD was reached.

Two different Trabedersen Treatment schedules (7-d-on, 7-d-off and 4-d-on, 10-d-off) were tested. Following completion of dose escalation another cohort of patients was enrolled for the treatment with one defined treatment schedule and dose to collect further safety and efficacy data in a larger group of patients.

Efficacy Assessments

Tumor size and response are determined through CT scan evaluation according to the RECIST criteria, version 1.0. Each change in tumor size as compared to baseline is classified into CR (complete response), PR (partial response), SD (stable disease) and PD (progressive disease).

The overall survival is calculated for all patients as the survival time from the onset of treatment with study medication to death due to any cause and analyzed with the Kaplan-Meier method.

Study Course and Efficacy Outcome Study Course

Altogether 33 patients with advanced pancreatic cancer, malignant melanoma, or colorectal cancer were enrolled for dose escalation (Table 15). Patients treated in the first treatment schedule received Trabedersen continuously for 7 days, followed by a treatment-free interval of 7 days for each treatment cycle (7-days-on, 7-days-off). The dose was successively increased from 40 to 80, 160, and 240 mg/m²/day. Three dose-limiting toxicities (2 thrombocytopenias, 1 exanthema) occurred with the dose of 240 mg/m²/day and established the MTD at 160 mg/m²/day in the 7-days-on, 7-days-off schedule.

After the MTD had been reached in the 7-days-on, 7-days-off schedule, a second dose escalation was started using a modified treatment schedule with 4 days Trabedersen administration, followed by a treatment-free interval of 10 days for each treatment cycle (4-days-on, 10-days-off). The dose was successively increased from 140 to 190, 250, and 330 mg/m²/day. As this modified treatment schedule proved to be well tolerated and as early signs of efficacy were seen already in the lowest dose group, dose escalation was stopped after the 4th cohort without reaching an MTD before the cumulative dose of the next dose level (440 mg/m²/day) would have exceeded the MTD-cumulative dose as established in the 7-days-on, 7-days-off schedule.

Subsequently, following the recommendations of the DSMB, a further cohort of 14 patients with advanced pancreatic cancer and 14 patients with malignant melanoma were enrolled and treated with a dose of 140 mg/m²/day Trabedersen within the 4-days-on, 10-days-off schedule.

A total of 61 patients were treated with Trabedersen. Of these, 50 patients completed the core study, i.e. received 2 cycles of Trabedersen, and a total of 42 patients participated in extension cycles. A summary of treatment schedule and patient disposition is given in Table 15.

TABLE 15
Treatment Schedule and Patient Disposition - Total Population

| Treatment | Dose | No. of patients | | Dose limiting toxicities |
|---|---|---|---|---|
| schedule | [mg/m²/day] | (PC/MM/CRC) | | (NCI-CTC grade) |
| 7-days-on, | 40 | 4 | (4/0/0) | — |
| 7-days-off | 80 | 3 | (2/1/0) | — |
| | 160 | 6 | (3/1/2) | — |
| | 240 | 4 | (2/0/2) | — 2 thrombocytopenias (3) |
| | | | | — 1 exanthema (3) |
| 4-days-on, | 140 | 5 | (5/0/0) | — 1 gastrointestinal bleeding (3) |
| 10-days-off | 190 | 3 | (2/1/0) | — |
| | 250 | 5 | (4/1/0) | — |
| | 330 | 3 | (1/1/1) | — |
| 4-days-on, | 140 | 28 | (14/14/0) | — |
| 10-days-off | | | | |
| (additional patients) | | | | |

| | | |
|---|---|---|
| Enrolled | 62 | (38/19/5) |
| Treated | 61 | (37/19/5) |
| Discontinued before start of study | 1 | (1/0/0) |
| Dropped out in core study | 11 | (7/1/3) |
| Completing core study | 50 | (30/18/2) |
| Participating in extension cycles | 42 | (25/16/1) |

CRC = Colorectal cancer,
MM = Malignant melanoma,
NCI-CTC = National Cancer Institute - Common Toxicity Criteria,
PC = Pancreatic Cancer

1.1.1.1.1 Patient Characteristics at Baselineen.

Table 16 shows patient demographic and baseline characteristics for the safety population (i.e. all patients that were treated with Trabedersen).

TABLE 16
Demographic and Baseline Characteristics - Safety Population

| | Pancreatic | | Malignant | | Colorectal | |
|---|---|---|---|---|---|---|
| | Cancer | | Melanoma | | Cancer | |
| | (N = 37) | | (N = 19) | | (N = 5) | |
| Gender n (%) | | | | | | |
| Male | 17 | (45.9%) | 8 | (42.1%) | 5 | (100%) |
| Female | 20 | (54.1%) | 11 | (57.9%) | 0 | (0%) |
| Median age range (years) | 63 | (40-76) | 61 | (44-74) | 61 | (43-67) |
| Race n (%) | | | | | | |
| Caucasian | 37 | (100%) | 19 | (100%) | 5 | (100%) |
| Previous radiation n (%) | 3 | (8.1%) | 5 | (26.3%) | 2 | (40.0%) |
| Previous surgery n (%) | 14 | (37.8%) | 19 | (100%) | 5 | (100%) |
| Previous chemotherapy n | 37 | (100%) | 19 | (100%) | 5 | (100%) |
| (%) | | | | | | |
| 1 | 16 | (43.2%) | 7 | (36.8%) | 0 | (0%) |
| 2 | 13 | (35.1%) | 9 | (47.4%) | 0 | (0%) |
| ≥3 | 8 | (21.6%) | 3 | (15.8%) | 5 | (100%) |
| Previous immunotherapy | 1 | (2.7%) | 14 | (73.7%) | 0 | (0%) |
| n (%) | | | | | | |
| Median time since first | 11.4 | | 65.6 | | 25.6 | |
| diagnosis [months] | | | | | | |
| KPS n (%) | | | | | | |
| 80 | 18 | (48.6%) | 1 | (5.3%) | 2 | (40.0%) |
| 90 | 13 | (35.1%) | 7 | (36.8%) | 2 | (40.0%) |
| 100 | 6 | (16.2%) | 11 | (57.9%) | 1 | (20.0%) |

N = Number of patients in the respective group;
n = number of patients with the respective characteristic;
KPS = Karnofsky Performance Status;
Percentages refer to “N”.
n.a. = not available

Survival and Antitumor Activity in Patients with Advanced Pancreatic Cancer

Overall Survival in the Treatment Schedules of the Dose Escalation

Altogether 21 patients with pancreatic cancer were treated during dose escalation. The median Overall Survival (mOS) of patients treated within the 7-days-on, 7-days-off schedule during dose escalation was comparable to the mOS of patients treated with the 4-days-on, 10-days-off schedule (5.7 months vs. 9.3 months, p=0.0645).

Overall Survival per Treatment Cohort

Table 17 shows the mOS per cohort of all 35 patients with pancreatic cancer treated. There was no clear dose-response relationship, neither in the 7-days-on, 7-days-off schedule nor in the 4-days-on, 10-days-off schedule. A similar pattern is seen when the 5 patients with malignant melanoma and 5 patients with colorectal cancer treated during dose-escalation are also included.

TABLE 17
Median Overall Survival per Dose Cohort

| | | | | Median Overall Survival in months | | | |
|---|---|---|---|---|---|---|---|
| | | | | [95% CI] | | | |
| Treatment | Dose | No. of patients | | Patients with | | | |
| schedule | [mg/m²/day] | (PC/MM/CRC) | | pancreatic cancer | | All patients | |
| 7-days-on, | 40 | 4 | (4/0/0) | 6.9 | [1.1, 11.1] | 6.9 | [1.1, 11.1] |
| 7-days-off | 80 | 2 | (1/1/0) | 4.6 | [ND, ND] | 9.2 | [4.6, 13.8] |
| | 160 | 6 | (3/1/2) | 3.9 | [3.2, 8.9] | 2.9 | [1.7, 8.9] |
| | 240 | 4 | (2/0/2) | 5.5 | [1.8, 9.2] | 6.5 | [1.8, 9.2] |
| 4-days-on, | 140 | 4 | (4/0/0) | 14.5 | [5.5, 39.7] | 14.5 | [5.5, 39.7] |
| 10-days-off | 190 | 3 | (2/1/0) | 6.2 | [3.0, 9.3] | 9.3 | [3.0, 11.4] |
| | 250 | 5 | (4/1/0) | 7.3 | [2.8, 16.1] | 9.8 | [2.8, 18.6] |
| | 330 | 3 | (1/1/1) | 2.4 | [ND, ND] | 3.0 | [2.4, ND] |
| 4-days-on, | 140 | 28 | (14/14/0) | 3.3 | [2.2, 5.5] | 6.0² | [4.6, 8.9] |
| 10-days-off | | | | | | | |
| (additional patients) | | | | | | | |

CI = confidence interval
CRC = Colorectal cancer,
MM = Malignant melanoma,
ND = not determined,
PC = Pancreatic cancer.

The 14 additional patients with pancreatic cancer treated with the 140 mg/m²/day dose in the 4-days-on, 10-days-off schedule had a lower mOS than the 4 patients with the same dose in the dose-escalation part of the study. However, these patients had an unfavorable prognosis as indicated by a long median time from first diagnosis (15.1 months), a high proportion of patients with a current diagnosis of AJCC Stage IV pancreatic cancer (86%), and a high proportion of patients receiving Trabedersen as 3^{rd}- or 4^{th}-line treatment (64%).

Overall Survival per Patient

Combining survival data from all 35 pancreatic carcinoma patients treated during dose escalation and in the last cohort, independent of Trabedersen dose and treatment schedule, resulted in an mOS of 4.9 months [95% CI: 3.0, 6.9 months]. Generally, patients receiving Trabedersen as 2^{nd}-line treatment had a better survival than patients receiving Trabedersen as 3^{rd}- to 4^{th}-line treatment: 11 of 17 patients (64.7%) who survived >5.0 months had received Trabedersen as 2nd-line treatment while only 4 of 18 patients (22.2%) who survived ≤5.0 months had received Trabedersen as 2^{nd}-line treatment. There was no obvious influence of baseline characteristics such as age, KPS, or disease duration on survival.

Overall Survival of Patients Treated 2^{nd}-Line with Trabedersen

In line with the finding that several patients treated 2^{nd}-line with Trabedersen showed a favorable survival, the mOS of all patients treated with Trabedersen as 2^{nd}-line therapy during the study independent of the dose and schedule was 8.9 months (95% CI: 2.9, 13.4). Restriction of the survival analysis to patients treated with the 140 mg/m²/day dose in the 4-days-on, 10-days-off schedule as 2nd-line treatment resulted in an mOS of 14.5 months (95% CI: 2.2, 18.9). Further sub-group analysis of patients treated with the 140 mg/m²/day dose in the 4-days-on, 10-days-off schedule as 2^{nd}-line treatment receiving subsequent chemotherapy after the end of Trabedersen treatment resulted in an mOS of 16.9 months (95% CI: 5.5, 39.7) compared to an mOS of 2.6 months (95% CI: 2.2, 2.9) in patient who did not receive subsequent chemotherapy after the end of Trabedersen treatment. Similar analysis of similar patient population treated with 5B1—an anti CA19 mAb—did not demonstrate the observed subsequent chemotherapy effect observed for Trabedersen.

Cytokine Profile Following Treatment with Trabedersen

An analyses of the effect from Trabedersen treatment on cyto-/chemokine levels was evaluated in 12 pancreatic cancer patients treated at 140 mg/m²/day on the 4-days-on, 10-days-off treatment schedule. A panel of 31 cyto-/chemokines were evaluated from clinical plasma samples over 3 cycles of Trabedersen at 8 separate timepoints (Baseline, Cycle 1 Day 2 and Day 5, Cycle 2 Day 1, Day 2 and Day 5, Final Visit, Cycle 3 Day 5). Cyto-/chemokine levels for each patient was standardized using log 10 transformed values calculated using the mean and standard deviation of each cyto-/chemokine within patients. To investigate the effect of Trabedersen on cyto-/chemokine levels and its correlation with OS, an ANCOVA model was developed.

The ANCOVA model was constructed such that at each of the cycle and timepoints, 2 variables (cyto-/chemokine, Overall Survival as a co-variate) and an interaction term (cyto-/chemokine×Overall Survival to profile the dependent variable response for each of the cyto-/chemokines and the Overall Survival) described changes in cyto-/chemokines and OS. Timepoints at which the model exhibited significant effects were further examined for the association of the cyto-/chemokine response and OS across the 12 patients. To test whether the assumptions of the model were satisfied, Normal-Quantile plots were examined for distribution of the residuals of the model. Significance of the relationship of the cyto-/chemokine and OS was assessed from the interaction term parameters and model error determination for each of the cyto/chemokines (P-values <0.05 were deemed significant if the false discovery rate was less than 10% considering all the relationships in the interaction term).

The developed ANCOVA model explained a significant proportion of the observed data for Cycle 1 Day 2 measurements of cyto-/chemokines (R²=0.3, F59,217=1.575, P<0.0103). Other timepoints did not exhibit a significant model fit and significant relationships in the interaction term (Baseline, R²=0.271, P =0.0542 (no significant relationships in the interaction term); Cycle 1 Day 5 R²=0.26, P=0.0984; Cycle 2 Day 1 R²=0.2, P=0.892; Cycle 2 Day 2 R²=0.26, P=0.368; Cycle 2 Day 5 R²=0.400, P=0.0256 (no significant relationships in the interaction term); Final Visit R²=0.170, P=0.996; Cycle 3 Day 5 R²=0.229, P=0.463).

Survival and Antitumor Activity in Patients with Advanced Melanoma and Colorectal Cancer

Five patients each with advanced malignant melanoma and colorectal cancer were enrolled into the dose escalation part of the study.

One patient with AJCC Stage IV colorectal cancer treated in the 240 mg/m²/day cohort of the 7-day-on, 7-day-off schedule was assessed with stable disease and survived for 7.3 months. The mOS of all patients independent of dose and schedule was 3.0 months (95% CI: 2.1, 7.3)

One patient with metastatic and dacarbazine (DTIC)-resistant melanoma treated in the 330 mg/m²/day cohort of the 4-day-on, 10-day-off schedule had stable disease and survived 25.7 months after start of study treatment. Further 3 patients with Stage IV melanoma survived for 11.4, 13.8 and 18.6 months (mOS of all patients: 13.8 months). All these patients had previously been treated with DTIC and PEG-Intron, i.e. received Trabedersen as 3^{rd}- or 4^{th}-line treatment.

Evaluation of 14 additional patients with malignant melanoma treated with 140 mg/m²/day in the 4-days-on, 10-days-off schedule showed a mOS of 10.4 months (95% CI: 5.4, 13.5). Survival between patients treated with the 7-days-on, 7-days-off schedule and the 4-days-on, 10-days-off schedule was not significantly different (7.8 months vs 11.4 months, p=0.501). At the time of database lock and final analysis, 4 patients were still alive with Overall Survival of 25.7, 13.8, 12.2, and 10.3 months. Overall survival of these 4 patients was censored during analysis, resulting in mOS 11.4 months (95% CI: 6.5, 13.8) for all patients independent of dose and schedule. Restricting survival analysis only to patients on the 4-days-on, 10-days-off schedule showed improved mOS in patients treated with subsequent therapies (chemotherapy or immunotherapy) compared to patients without (13.5 months vs 6.0 months). There was an even distribution of treatment with immunotherapy or chemotherapy only (4 patients vs 3 patients) or a combination of both (4 patients). Further limiting analysis to patients in the last cohort (140 mg/m2/day treated 4-days-on, 10-days-off) revealed significant improvements in mOS (13.5 months vs 6.0 months, p=0.0015) when trabedersen was followed by subsequent therapy.

Two melanoma patients were treated 2^{nd}-line with 140 mg/m²/day in the 4-days-on, 10-days-off schedule and showed a mOS of 9.5 months (95% CI: 5.4, 13.5).

### Example 15—Antiviral Activity of Oncotelic Compounds vs Sudden Acute Respiratory Syndrome-Associated Coronaviruses Procedure

OT-101 (Trabedersen) and the ten antisense compounds were received from sponsor in lyophilized form. Compounds were solubilized in sterile saline to prepare 20 mg/mL stock solutions which were sterile filtered through a 0.2 μM low protein binding filter. Compounds were serially diluted using eight half-log dilutions in test medium (MEM supplemented with 2% FBS and 50 mg/mL gentamicin) so that the starting (high) test concentration was 1000 mg/mL. Each dilution was added to 5 wells of a 96-well plate with 80-100% confluent Vero 76 cells. Three wells of each dilution were infected with virus, and two wells remained uninfected as toxicity controls. Six wells were infected and untreated as virus controls, and six wells were uninfected and untreated as cell controls. SARS-CoV and SARS-CoV-2 virus suspensions were prepared to achieve the lowest possible multiplicity of infection (MOI) that would yield >80% cytopathic effect (CPE) within 5 days. M128533 was tested in parallel as a positive control. Plates were incubated at 37±2° C., 5% CO2.

On day 5 post-infection, once untreated virus control wells reached maximum CPE, plates were stained with neutral red dye for approximately 2 hours (±15 minutes). Supernatant dye was removed and wells rinsed with PBS, and the incorporated dye was extracted in 50:50 Sorensen citrate buffer/ethanol for >30 minutes and the optical density was read on a spectrophotometer at 540 nm. Optical densities were converted to percent of cell controls and normalized to the virus control, then the concentration of test compound required to inhibit CPE by 50% (EC50) was calculated by regression analysis. The concentration of compound that would cause 50% cell death in the absence of virus was similarly calculated (CC50). The selective index (SI) is the CC50 divided by EC50.

Results

Antiviral activity against SARS-CoV for each compound is shown in Table 1. Cytotoxicity was observed for TRS2 (56-76) and 5TERM (1-20) MOE and OT-101 exhibited moderate antiviral activity. The positive control compound performed as expected.

Antiviral activity against SARS-CoV-2 for each compound is shown in Table 2. Cytotoxicity was observed for TRS1 (53-72), FS (13,458-13,472), and 5TERM (1-20) MOE. High antiviral activity was observed with the following compounds: OT-101, 5TERM (1-20), TRS1 (53-72), FS (13,458-13,472), 5TERM (1-20) MOE, TRS2-2 53-72, FS-2a (13539-13558), and artemisinin. The positive control compound performed as expected.

TABLE 18
In vitro antiviral activity of Onctotelic
compounds against SARS-CoV.

| | EC₅₀ | CC₅₀ | SI |
|---|---|---|---|
| OT-101 | 26 | >1000 | >38 |
| 5TERM (1-20) | >1000 | >1000 | 0 |
| TRS1 (53-72) | >1000 | >1000 | 0 |
| TRS2 (56-76) | 340 | >1000 | >2.9 |
| FS (13,458-13,472) | >1000 | >1000 | 0 |
| 5TERM (1-20) MOE | 380 | >1000 | >2.6 |
| TRS2-2 53-72 | >1000 | >1000 | 0 |
| TRS2-2 53-72 MOE | >1000 | >1000 | 0 |
| FS (13,458-13,274)MOE | >1000 | >1000 | 0 |
| FS-2a (13539-13558) | >1000 | >1000 | 0 |
| RSV1 | >1000 | >1000 | 0 |
| M128533 (positive control) | 0.16 | >100 | >630 |

Units are in mg/mL for test compounds and
   M128533 EC₅₀: 50% effective antiviral concentration
CC₅₀: 50% cytotoxic concentration of compound without virus added SI=CC₅₀/EC₅₀

TABLE 19
In vitro antiviral activity of Onctotelic
compounds against SARS-CoV-2.

| | EC₅₀ | CC₅₀ | SI |
|---|---|---|---|
| OT-101 | 2.0 | >1000 | >500 |
| 5TERM (1-20) | 7.1 | >1000 | >140 |
| TRS1 (53-72) | 7.6 | 720 | 95 |
| TRS2 (56-76) | 73 | >1000 | >14 |
| FS (13,458-13,472) | 5.2 | 430 | 53 |
| 5TERM (1-20) MOE | 4.9 | 610 | 120 |
| TRS2-2 53-72 | 1.9 | >1000 | >530 |
| TRS2-2 53-72 MOE | 62 | >1000 | >16 |
| FS (13,458-13,274)MOE | 25 | >1000 | >40 |
| FS-2a (13539-13558) | 17 | >1000 | >59 |
| RSV1 | 620 | >1000 | >1.6 |
| Artemisinin | 0.45 | 61 | 140 |
| M128533 (positive control) | 0.012 | >10 | >830 |

Units are in mg/mL for test compounds and M128533 EC50: 50% effective antiviral concentration
CC₅₀: 50% cytotoxic concentration of compound without virus added SI=CC₅₀/EC₅₀

### Example 16-OT-101 Treatment Suppressed IL-6

Cytokine levels of clinical plasma samples of pancreatic cancer patients of the P001 study of OT-101 in advanced solid tumor patients were measured using the ImmunoSignal cytokine storm assay developed by Eurofins.
Nine patients with elevated IL-6 were examined further. More than 50% of these patients (6 of 9) exhibited significant reduction in IL-6 level following 1st cycle of dosing with OT-101. Of significant are pts 1041 and 1051 who exhibited a rebound following treatment stop on cycle 1 which decreased again on subsequent cycle 2. All patients exhibited elevated IL-6 on disease progression.
   Various modifications of the invention, in addition to those described herein will be apparent to those skilled in the art from the foregoing description. Such modifications are also intended to fall within the scope of the appended claims. Each reference cited in the present application, including all patent, patent applications and publications, is incorporated herein by reference in its entirety.

### REFERENCES

Schuftan C. A story to be shared: the successful fight against malaria in Vietnam. WHO WPRO and the global Roll Back Malaria Programme, 2000 https://www.panna.org/sites/default/files/vietnam Malara Study 20 071106.

Li Q, Weina P J. Severe embryotoxicity of artemisinin derivatives in experimental animals, but possibly safe in pregnant women. Molecules. 2009; 15(1):40-57.

Kovacs S D, Rijken M J, Stergachis A. Treating severe malaria in pregnancy: a review of the evidence. Drug Saf. 2015; 38(2):165-181.

Nosten F, McGready R, d'Alessandro U, et al. Antimalarial drugs in pregnancy: a review Curr Drug Saf. 2006; 1(1):1-15.

Clark R L. Embryotoxicity of the artemisinin antimalarials and potential consequences for use in women in the first trimester. Reprod Toxicol. 2009; 28(3):285-296.

Guidelines for the Treatment of Malaria. 2nd ed. Geneva: World Health Organization; 2010.

Visser B J, van Vugt M, Grobusch M P. Malaria: an update on current chemotherapy. Expert Opin Pharmacother. 2014; 15(15):2219-2254.

McGready R, Lee S J, Wiladphaingern J, et al. Adverse effects of falciparum and vivax malaria and the safety of antimalarial treatment in early pregnancy: a population-based study. Lancet Infect Dis. 2012; 12(5):388-396.

Savage R L, Hill G R, Barnes J, Kenyon S H, Tatley M V. Suspected Hepatotoxicity With a Supercritical Carbon Dioxide Extract of Artemisia annua in Grapeseed Oil Used in New Zealand. Front Pharmacol. 2019; 10:1448.

Barnes J. Pharmacovigilance of herbal medicines: a UK perspective. Drug Saf. 2003; 26(12):829-851.

Uppsala Monitoring Centre. VigiBase: Uppsala Monitoring Centre. 2019. Available from: https://www.who-umc.org/vigibase/vigibase/.

Uppsala Monitoring Centre. VigiAccess: Uppsala Monitoring Centre. 2019. Available from: http://www.vigiaccess.org/.

Centers for Disease Control and Prevention (CDC). Hepatitis temporally associated with an herbal supplement containing artemisinin—Washington, 2008. MMWR Morb Mortal Wkly Rep. 2009; 58(31):854-856.

Kumar S. Cholestatic liver injury secondary to artemisinin. Hepatology. 2015; 62(3):973-974.

US Food and Drug Administration. “Pharmacy Compounding Advisory Committee,” in Compounders under section 503A of the FD&C act: quality, standards and FDA findings., 2017; 31-43.

Stebbings S, Beattie E, McNamara D, Hunt S. A pilot randomized, placebo-controlled clinical trial to investigate the efficacy and safety of an extract of Artemisia annua administered over 12 weeks, for managing pain, stiffness, and functional limitation associated with osteoarthritis of the hip and knee. Clin Rheumatol. 2016; 35(7):1829-1836.

Hunt S, Stebbings S, McNamara D. An open-label six-month extension study to investigate the safety and efficacy of an extract of Artemisia annua for managing pain, stiffness and functional limitation associated with osteoarthritis of the hip and knee. N Z Med J. 2016; 129(1444):97-102.

Heinrich M., Barnes J., Gibbons S., Prieto J., Williamson E. Methods in natural product analytical chemistry. In Fundamentals of Pharmacognosy and Phytotherapy, 3rd edition. Edinburgh: Elsevier. 2018; 106.

Zhang X, Zhao Y, Guo L, Qiu Z, Huang L, Qu X. Differences in chemical constituents of Artemisia annua L from different geographical regions in China. PLoS One. 2017; 12(9):e0183047.

DerMarderosian A., Beutler J. A., (eds). The Review of Natural Products. 8th ed. St. Louis, Mo.: Clinical Drug Information, LLC. 2014.

Iqbal S, Younas U, Chan K W, Zia-Ul-Haq M, Ismail M. Chemical composition of Artemisia annua L. leaves and antioxidant potential of extracts as a function of extraction solvents. Molecules. 2012; 17(5):6020-6032.

Promisia. Arthrem®. 2019. Available from: http://arthrem.co.nz/Arthrem/Product.

GO Healthy. GO Arthri Remedy 1-A-Day. 2019. Available from: https://www.healthporter.co.nz/go-healthy-go-arthri-remedy-1-a-day-60-capsules.

Sehailia M, Chemat S. In-silico Studies of Antimalarial-agent Artemisinin and Derivatives Portray More Potent Binding to Lys353 and Lys31-Binding Hotspots of SARS-CoV-2 Spike Protein than Hydroxychloroquine: Potential Repurposing of Artenimol for COVID-19. ChemRxiv. 2020. Preprint.

Alazmi M, Motwalli O. Molecular basis for drug repurposing to study the interface of the S protein in SARS-CoV-2 and human ACE2 through docking, characterization, and molecular dynamics for natural drug candidates. J Mol Model. 2020; 26:338.

Cao Y, Feng Y H, Gao L W, et al. Artemisinin enhances the anti-tumor immune response in 4T1 breast cancer cells in vitro and in vivo. Int Immunopharmacol. 2019; 70:110-116.

Wang M, Cao R, Zhang L, et al. Remdesivir and chloroquine effectively inhibit the recently emerged novel coronavirus (2019-nCoV) in vitro. Cell Res. 2020; 30(3):269-271.

Cao R, Hu H, Li Y, et al. Anti-SARS-CoV-2 Potential of Artemisinins In Vitro. ACS Infect Dis. 2020; 6(9):2524-2531.

Gilmore K, Zhou Y, Ramirez S, et al. In vitro efficacy of Artemisinin-based treatments against SARS-CoV-2. bioRxiv. 2020. 10.05.326637.

Li G, Yuan M, Li H, et al. Safety and efficacy of artemisinin-piperaquine for treatment of COVID-19: an open-label, non-randomised and controlled trial. Int J Antimicrob Agents. 2020; 106216.

MGC Pharmaceutical Ltd. ArtemiC™ Phase II Clinical Trial Results on COVID-19 patients confirm 100% of treatment group successfully met primary and secondary endpoints. Press Release. 14 Dec. 2020.

Liu, J. et al. Hydroxychloroquine, a less toxic derivative of chloroquine, is effective in inhibiting SARS-CoV-2 infection in vitro. Cell. Discov. 6, 16 (2020).

Yao, X. et al. In Vitro antiviral activity and projection of optimized dosing design of hydroxychloroquine for the treatment of severe acute respiratory syndrome coronavirus 2 (SARSCoV-2). Clin. Infect. Dis., (2020) doi: 10.1093/cid/ciaa237.

Al-Kofahi M. et al. Finding the dose for hydroxychloroquine prophylaxis for COVID-19; the desperate search for effectiveness. Clinical Pharmacology & Therapeutics. (2020) (In press).https://doi.org/10.1002/cpt.1874.

CHEN J. et al. A pilot study of hydroxychloroquine in treatment of patients with common coronavirus disease-19 (COVID-19). J Zhejiang Univ (Med Sci) 49, 0-(2020).

Gautret, P. et al. Hydroxychloroquine and azithromycin as a treatment of COVID-19: results of an open-label non-randomized clinical trial. Int. J. Antimicrob. Agents. 105949 (2020) doi:10.1016/j.ijantimicag.2020.105949.

Wang M. et al. Remdesivir and chloroquine effectively inhibit the recently emerged novel coronavirus (2019-nCoV) in vitro. Cell Res 2020; 30:269-71.

Wang Y. et al. Remdesivir in adults with severe COVID-19: a randomised, double-blind, placebo-controlled, multicentre trial. Lancet (2020) https://doi.org/10.1016/S0140-6736(20)31022-9

Zhou F et al. Clinical course and risk factors for mortality of adult inpatients with COVID-19 in Wuhan, China: a retrospective cohort study. Lancet 2020; 395:1054-62 9. Hamacher J, Lucas R, Lijnen H R, Buschke S, Dunant Y, Wendel A, et al. Tumor necrosis factoralpha and angiostatin are mediators of endothelial cytotoxicity in bronchoalveolar lavages of

patients with acute respiratory distress syndrome. Am J Respir Crit Care Med (2002) 166:651-6. doi:10.1164/rccm.2109004

Wagener B M, Roux J, Caries M, Pittet J F. Synergistic inhibition of beta2-adrenergic receptormediated alveolar epithelial fluid transport by interleukin-8 and transforming growth factor-beta. Anesthesiology (2015)122:1084-92. doi:10.1097/ALN.0000000000000595

Fahy R J, Lichtenberger F, McKeegan C B, Nuovo G J, Marsh C B, Wewers M D. The acute respiratory distress syndrome: a role for transforming growth factor-beta 1. Am J Respir Cell Mol Biol (2003) 28:499-503. doi:10.1165/rcmb.2002-0092OC.

Wakefield L M, Letterio J J, Chen T, Danielpour D, Allison R S, Pal L H, et al. Transforming growth factor-beta1 circulates in normal human plasma and is unchanged in advanced metastatic breast cancer. Clin Cancer Res (1995)1:129-36.

Schwartze J T, Becker S, Sakkas E, Wujak L A, Niess G, Usemann J, et al. Glucocorticoids recruit Tgfbr3 and Smad1 to shift transforming growth factor-beta signaling from the Tgfbr1/Smad2/3 axis to the Acvrl1/Smad1 axis in lung fibroblasts. J Biol Chem (2014) 289:3262-75. doi:10.1074/jbc.M113.541052.

Matsuki K, Hathaway C K, Lawrence M G, Smithies O, Kakoki M. The role of transforming growth factor betal in the regulation of blood pressure. Curr Hypertens Rev (2014) 10:223-38. doi:10.2174/157340211004150319123313.

Kaminski N, Allard J D, Pittet J F, Zuo F, Griffiths M J, Morris D, et al. Global analysis of gene expression in pulmonary fibrosis reveals distinct programs regulating lung inflammation and fibrosis. Proc Natl Acad Sci U S A (2000) 97:1778-83. doi:10.1073/pnas.97.4.1778.

Annes J P, Chen Y, Munger J S, Rifkin D B. Integrin alphaVbeta6-mediated activation of latent TGF-beta requires the latent TGF-beta binding protein-1. J Cell Biol (2004) 165:723-34. doi: 10.1083/jcb.200312172.

Annes J P, Rifkin D B, Munger J S. The integrin alphaVbeta6 binds and activates latent TGFbeta3.FEBS Lett (2002) 511:65-8. doi:10.1016/S0014-5793(01)03280-X.

Munger J S, Huang X, Kawakatsu H, Griffiths M J, Dalton S L, Wu J, et al. The integrin alpha v beta 6 binds and activates latent TGF beta 1: a mechanism for regulating pulmonary inflammation and fibrosis. Cell (1999) 96:319-28. doi:10.1016/S0092-8674(00)80545-0.

Breuss J M, Gallo J, DeLisser H M, Klimanskaya I V, Folkesson H G, Pittet J F, et al. Expression of the beta 6 integrin subunit in development, neoplasia and tissue repair suggests a role in epithelial remodeling. J Cell Sci (1995) 108(Pt 6):2241-51.

Barcellos-Hoff M H, Dix T A. Redox-mediated activation of latent transforming growth factorbeta 1. Mol Endocrinol (1996) 10:1077-83. doi:10.1210/me.10.9.1077.

Frank J, Roux J, Kawakatsu H, Su G, Dagenais A, Berthiaume Y, et al. Transforming growth factor-betal decreases expression of the epithelial sodium channel alphaENaC and alveolar epithelial vectorial sodium and fluid transport via an ERK1/2-dependent mechanism. J Biol Chem (2003) 278:43939-50. doi:10.1074/jbc.M304882200.

Pittet J F, Griffiths M J, Geiser T, Kaminski N, Dalton S L, Huang X, et al. TGF-beta is a critical mediator of acute lung injury. J Clin Invest (2001) 107:1537-44. doi:10.1172/JCI11963.

Peters D M, Vadasz I, Wujak L, Wygrecka M, Olschewski A, Becker C, et al. TGF-beta directs trafficking of the epithelial sodium channel ENaC which has implications for ion and fluid transport in acute lung injury. Proc Natl Acad Sci USA (2014) 111:E374-83. doi:10.1073/pnas.1306798111.

Frank J A, Matthay M A. TGF-beta and lung fluid balance in ARDS. Proc Natl Acad Sci USA(2014) 111:885-6. doi:10.1073/pnas.1322478111.
Staub N C. Pulmonary edema: physiologic approaches to management. Chest. 1978; 74(5):559-564.

Prewitt R M, McCarthy J, Wood L D. Treatment of acute low pressure pulmonary edema in dogs:relative effects of hydrostatic and oncotic pressure, nitroprusside, and positive end-expiratory pressure. J Clin Invest. 1981; 67(2):409-418.

Wiedemann H P, et al. Comparison of two fluid management strategies in acute lung injury. N Engl J Med. 2006; 354(24):2564-2575.

Wheeler A P, et al. Pulmonary-artery versus central venous catheter to guide treatment of acute lung injury. N Engl J Med. 2006; 354(21):2213-2224.

Pittet J F, Griffiths M J, Geiser T, Kaminski N, Dalton S L, Huang X, et al. TGF-beta is a critical mediator of acute lung injury. J Clin Invest (2001) 107(12):1537-44. doi:10.1172/jci11963.

Hu X, Huang X. Alleviation of Inflammatory Response of Pulmonary Fibrosis in Acute Respiratory Distress Syndrome by Puerarin via Transforming Growth Factor (TGF-b1). Med Sci Monit, 2019; 25: 6523-6531.

Mizutani T, Fukushi S, Iizuka D, Inanami O, Kuwabara M, Takashima H, Yanagawa H, Saijo M, Kurane I, Morikawa S. Inhibition of cell proliferation by SARS-CoV infection in Vero E6 cells.FEMS Immunol Med Microbiol. 2006; 46(2):236-243.

Surjit M, Liu B, Chow V T, Lal S K. The nucleocapsid protein of severe acute respiratory syndrome-coronavirus inhibits the activity of cyclin-cyclin-dependent kinase complex and blocks S phase progression in mammalian cells. J. Biol. Chem. 2006; 281(16):10669-10681.

Dove B, Brooks G, Bicknell K, Wurm T, Hiscox J A. Cell cycle perturbations induced by infection with the coronavirus infectious bronchitis virus and their effect on virus replication. J. of Virology. 2006; 80(8): 4147-4156.

Huang K J, Su I J, Theron M, Wu Y C, Lai S K, Liu C C, Lei H Y. An interferon-gamma related cytokine storm in SARS patients. J. Med. Virol. 2005; 75(2):185-194.

He L, Ding Y, Zhang Q, Che X, He Y, Shen H, Wang H, Li Z, Zhao L, Geng J, Deng Y, Yang L, Li J, Cai J, Qiu L, Wen K, Xu X, Jiang S. Expression of elevated levels of pro-inflammatory cytokines in SARS-CoV infected ACE2+cells in SARS patients: relation to the acute lung injury and pathogenesis of SARS. J. Pathol. 2006; 210(3):288-297.

Zhao X, Nicholls J M, Chen Y G. Severe acute respiratory syndrome-associated coronavirus nucleocapsid protein interacts with Smad3 and modulates transforming growth factor-beta signalling. J. Biol. Chem. 2008; 283(6):3272-3280.

Li S W, Yang T C, Wan L, Lin Y J, Tsai F J, Lai C C, Lin C W. Correlation between TGF-β1 expression and proteomic profiling induced by severe acute respiratory syndrome coronavirus papain-like protease. Proteomics. 2012; 12(21):3193-3205.

Wang C Y, Lu C Y, Li S W, Lai C C, Hua C H, Huang S H, Lin Y J, Hour M J, Lin C W. SAR coronavirus papain-like protease up-regulates the collagen expression through non-Samd TGF-β1 signaling. Virus Research. 2017; 235:58-66.

Li S W, Wang C Y, Jou Y J, Yang T C, Huang S H, Wan L, Lin Y J, Lin C W. SARS coronavirus papain-like protease induces Egr-1-dependent up-regulation of TGF-β1 via ROS/p38 MAPK/STAT3 pathway. Scientific Reports. 2016; 6:25754.

Pang B S, Wang Z, Zhang L M, Tong Z H, Xu L L, Huang X X, Guo W J, Zhu M, Wang C, Li X W et al (2003) Dynamic changes in blood cytokine levels as clinical indicators in severe acute respiratory syndrome. Chin Med J 116:1283-1287.

Baas T, Taubenberger J K, Chong P Y, Chui P, Katze M G (2006) SARS-CoV virus-host interactions and comparative etiologies of acute respiratory distress syndrome as determined by transcriptional and cytokine profiling of formalin-fixed paraffin-embedded tissues. J Interferon Cytokine Res 26:309-317.

Chen X. et al. Detectable serum SARS-CoV-2 viral load (RNAaemia) is closely associated with drastically elevated interleukin 6 (IL-6) level in critically ill COVID-19 patients. medRxiv preprint doi: htts://doi.org/10.1101/2020.02.29.20029520.

Cao Y. et al. Artemisinin enhances the anti-tumor immune response in 4T1 breast cancer cells in vitro and in vivo. Int. Immunopharmacology 70 (2019) 110-116.

Zheng S., J. Yang, X. Hu, M. Li, Q. Wang, R. C. A. Dancer, D. Parekh, F. Gao-Smith, D. R.

Sazani P, Kole R. Therapeutic potential of antisense oligonucleotides as modulators of alternative splicing. J Clin Invest. 2003; 112(4):481-486.

Juliano R, Alam M R, Dixit V, Kang H. Mechanisms and strategies for effective delivery of antisense and siRNA oligonucleotides. Nucleic Acids Res. 2008; 36(12):4158-4171.

Chan J, Lim S, Wong W S. Antisense oligonucleotides: from design to therapeutic applications. Clin Exp Pharmacol Physiol. 2006; 33(5-6):533-540.

Kurreck J. Antisense technologies. Improvement through novel chemical modifications. Eur J Biochem. 2003; 270(8):1628-1644.

Crooke S T. Progress in antisense technology. Annu Rev Med. 2004; 55:61-95.

Ratmeyer L, Vinayak R, Zhong Y Y, Zon G, Wilson W D. Sequence specific thermodynamic and structural properties for DNA.RNA duplexes. Biochemistry. 1994; 33(17):5298-5304.

Tu G C, Cao Q N, Zhou F, Israel Y. Tetranucleotide GGGA motif in primary RNA transcripts. Novel target site for antisense design. J Biol Chem. 1998; 273(39): 25125-25131.

Benimetskaya L, Berton M, Kolbanovsky A, Benimetsky S, Stein C A. Formation of a G-tetrad and higher order structures correlates with biological activity of the RelA (NF-kappaB p65) ‘antisense’ oligodeoxynucleotide. Nucleic Acids Res. 1997; 25(13): 2648-2656.

Wang W, Chen H J, Sun J, Benimetskaya L, Schwartz A, Cannon P, Stein C A, Rabbani L E. A comparison of guanosine-quartet inhibitory effects versus cytidine homopolymer inhibitory effects on rat neointimal formation. Antisense Nucleic Acid Drug Dev. 1998; 8(3):227-236.

Williamson J R, Raghuraman M K, Cech T R. Monovalent cation-induced structure of telomeric DNA: the G-quartet model. Cell. 1989; 59(5):871-880.

Schultze P, Macaya R F, Feigon J. Three-dimensional solution structure of the thrombin-binding DNA aptamer d(GGTTGGTGTGGTTGG). J Mol Biol. 1994; 235(5): 1532-1547.

Chou S H, Zhu L, Reid B R. The unusual structure of the human centromere (GGA)2 motif. Unpaired guanosine residues stacked between sheared G.A pairs. J Mol Biol. 1994; 244(3): 259-268.

Griffin L C, Toole J J, Leung L L. The discovery and characterization of a novel nucleotide-based thrombin inhibitor. Gene. 1993; 137(1):25-31.

Wyatt J R, Vickers T A, Roberson J L, Buckheit R W Jr, Klimkait T, DeBaets E, Davis P W, Rayner B, Imbach J L, Ecker D J. Combinatorially selected guanosine-quartet structure is a potent inhibitor of human immunodeficiency virus envelope-mediated cell fusion. Proc Natl Acad Sci USA. 1994; 91(4):1356-1360.

Tam R C, Lin C J, Lim C, Pai B, Stoisavljevic V. Inhibition of CD28 expression by oligonucleotide decoys to the regulatory element in exon 1 of the CD28 gene. J Immunol. 1999; 163(8): 4292-4299.

Bates P J, Kahlon J B, Thomas S D, Trent J O, Miller D M. Antiproliferative activity of G-rich oligonucleotides correlates with protein binding. J Biol Chem. 1999; 274(37): 26369-26377.

Murchie A I, Lilley D M. Tetraplex folding of telomere sequences and the inclusion of adenine bases. EMBO J. 1994; 13(4): 993-1001.

Olivas W M, Maher L J 3rd. Overcoming potassium-mediated triplex inhibition. Nucleic Acids Res. 1995; 23(11):1936-1941.

Stein C A, Tonkinson J L, Yakubov L. Phosphorothioate oligodeoxynucleotides—anti-sense inhibitors of gene expression? Pharmacol Ther. 1991; 52(3):365-384.

Crooke S T, Lebleu B, eds. Antisense Research and Applications. Boca Raton Fla.: CRC Press. 1993.

Srinivasan S K, Iversen P. Review of in vivo pharmacokinetics and toxicology of phosphorothioate oligonucleotides. J Clin Lab Anal. 1995; 9(2):129-137.

Bonham M A, Brown S, Boyd A L, Brown P H, Bruckenstein D A, Hanvey J C, Thomson S A, Pipe A, Hassman F, Bisi J E, Froehler B C, Matteucci M D, Wagner R W, Nobel S A, Babiss L E. An assessment of the antisense properties of RNase H-competent and steric-blocking oligomers. Nucleic Acids Res. 1995; 23(7):1197-1203.

Yaida Y, Nowak T S Jr. Distribution of phosphodiester and phosphorothioate oligonucleotides in rat brain after intraventricular and intrahippocampal administration determined by in situ hybridization. Regul Pept. 1995; 59(2):193-199.

Crooke S T, Lemonidis K M, Neilson L, Griffey R, Lesnik E A, Monia B P. Kinetic characteristics of Escherichia coli RNase H1: cleavage of various antisense oligonucleotide-RNA duplexes. Biochem J. 1995; 312(Pt 2):599-608.

Gura T. Antisense has growing pains. Science. 1995; 270(5236):575-577.

Krieg A M, Stein C A. Phosphorothioate oligodeoxynucleotides: antisense or anti-protein? Antisense Res Dev. 1995; 5(4):241.

Krieg A M, Yi A K, Hartmann G. Mechanisms and therapeutic applications of immune stimulatory cpG DNA. Pharmacol Ther. 1999; 84(2):113-120.

Lipford G B, Bauer M, Blank C, Reiter R, Wagner H, Heeg K. CpG-containing synthetic oligonucleotides promote B and cytotoxic T cell responses to protein antigen: a new class of vaccine adjuvants. Eur J Immunol. 1997; 27(9):2340-2344.

Sun S, Zhang X, Tough D F, Sprent J. Type I interferon-mediated stimulation of T cells by CpG DNA. J Exp Med. 1998; 188(12):2335-2342.

Bendigs S, Salzer U, Lipford G B, Wagner H, Heeg K. CpG-oligodeoxynucleotides co-stimulate primary T cells in the absence of antigen-presenting cells. Eur J Immunol. 1999; 29(4): 1209-1218.

Jakob T, Walker P S, Krieg A M, von Stebut E, Udey M C, Vogel J C. Bacterial DNA and CpG-containing oligodeoxynucleotides activate cutaneous dendritic cells and induce IL-12 production: implications for the augmentation of Thl responses. Int Arch Allergy Immunol. 1999; 118(2-4):457-461.

Krieg A M, Matson S, Fisher E. Oligodeoxynucleotide modifications determine the magnitude of B cell stimulation by CpG motifs. Antisense Nucleic Acid Drug Dev. 1996; 6(2): 133-139.

1-79. (canceled)

80. A method for treating or ameliorating a viral disease due to SARS-CoV-2 in a patient in need, the method comprising administering to the patient a composition comprising an agent for inhibiting or suppressing expression of TGF-β.

81. The method of claim 80, wherein the method ameliorates one or more symptoms comprising cytokine storm, multiorgan inflammatory syndrome, Kawasaki syndrome, IgA vasculitis, cytokine induced pneumonia, or suppresses TGF-β-induced proteins.

82. The method of claim 80, wherein the SARS-CoV-2 is any variant of COVID-19.

83. The method of claim 80, wherein the route of administration is intravenous, intrathecal, intramuscular, subcutaneous, or oral.

84. The method of claim 80, wherein the agent for inhibiting or suppressing expression of TGF-β is an antisense oligonucleotide.

85. The method of claim 84, wherein the antisense oligonucleotide is selected from SEQ ID NOs:5-13 as follows
SEQ ID NO: 5,
gtaggtaaaa acctaatat
SEQ ID NO: 6,
gttcgtttag agaacagatc
SEQ ID NO: 7,
taaagttcgt ttagagaaca g
SEQ ID NO: 8,
agccctgtat acgac
SEQ ID NO: 9,
gtaggtaaaa acctaatat
SEQ ID NO: 10,
cgtttagaga acagatctac
SEQ ID NO: 11,
cattgtagat gtcaaaagcc
SEQ ID NO: 12,
ctccctcatg gtggcagttg a
SEQ ID NO: 13,
cggcatgtct attttgta (OT-101)
and chemically-modified variants thereof, LNA variants thereof, gapmer variants thereof, and any combination or pooling thereof.

86. The method of claim 84, wherein the antisense oligonucleotide is SEQ ID NO:13 cggcatgtct attttgta (OT-101) and chemically-modified variants thereof, LNA variants thereof, gapmer variants thereof, and any combination or pooling thereof.

87. The method of claim 84, wherein the antisense oligonucleotide is in a sterile saline solution at a concentration of from 1000 μg/mL to 20 mg/mL.

88. The method of claim 80, wherein the agent for inhibiting or suppressing expression of TGF-β comprises an Artemisia annua extract.

89. The method of claim 88, wherein the Artemisia annua extract is at least 90% pure Artemisinin, and pharmaceutically acceptable salts, esters, polymorphs, stereoisomers, and mixtures thereof.

90. The method of claim 88, wherein the Artemisia annua extract comprises Artemisinin in an amount of 250-750 mg.

91. The method of claim 88, wherein the Artemisia annua extract comprises an oral dosage form comprising Artemisinin in capsules, tablets, powders, pouches, sachets, or suppositories.

92. The method of claim 88, wherein the Artemisia annua extract is substantially free of Artemisitene, 9-epiartemisinin, and Thujone.

93. The method of claim 88, wherein the Artemisia annua extract comprises one or more of artemether (ARM), artesunate (ARS), and dihydroartemisinin.

94. The method of claim 88, wherein the Artemisia annua extract comprises 45-99% w/w of Artemisinin.

95. The method of claim 88, wherein the Artemisia annua extract comprises 88-97 weight % of Artemisinin.

96. The method of claim 88, wherein the Artemisia annua extract is formulated with one or more pharmaceutically-acceptable excipients selected from diluents, stabilizers, disintegrants, and anticaking agents.

97. The method of claim 88, wherein the Artemisia annua extract is formulated with 1-5 weight % of stabilizers, 0.2-1 weight % of diluents, 1-4 weight % of disintegrants, and 1-2 weight % of anticaking agents.

98. The method of claim 88, wherein the Artemisia annua extract is formulated with stabilizer polysorbate 80, diluent microcrystalline cellulose, disintegrant crospovidone or croscarmellose, and anticaking agent magnesium stearate.

99. The method of claim 88, wherein the Artemisia annua extract is formulated with one or more of Curcumin, Boswellia, Vitamin C, Piperiquine, and Pyronaridine.
